(19) United States
(12) Patent Application Publication
(10) Pub. No.: US 2007/0099304 A1
(43) Pub. Date: May 3, 2007

(54) Method for the selective isolation of multiply-charged peptides applicable in the quantitative proteomics

(76) Inventors: Aniel Sanchez Puente, Ciudad de la Habana (CU); Luis Javier Gonzalez Lopez, Ciudad de la Habana (CU); Joevanis Gil Valdes, Ciudad de la Habana (CU); Lazaro Hiram Betancourt Nunes, Cuidad de la Habana (CU); Vladimir Armando Besada Perez, Cuidad de la Habana (CU); Jorge Fernandez de Cossio Dorta-Duque, Ciudad de la Habana (CU); Felix Modesto Alvarez Gil, Ciudad de la Habana (CU); Gabriel Ramon Padron Palomares, La Habana (CU)

Correspondence Address: HOFFMANN & BARON, LLP, 6900 JERICHO TURNPIKE, SYOSSET, NY, 11791, US

(21) Appl. No.: 11/582,525

(22) Filed: Oct. 17, 2006

(30) Foreign Application Priority Data
Oct. 17, 2005 (CU) CU-2005-0197

(51) Int. Cl. G01N 33/00

(52) U.S. Cl. 436/86

(57) ABSTRACT

The present invention describes a method that combines the blocking of primary amino groups and the cation exchange chromatography, to simplify peptide mixtures that can be generated or not by proteolytic or chemical treatments. This method allows the selective isolation of an average of 4 multiply-charged peptides (RH peptides) per protein and the study of 90% of the proteins of the analyzed proteomes. It is applicable to studies of quantitative proteomics without the usage of two-dimensional electrophorsis, it is compatible with any type of isotopic labeling and it is very useful to determine the differential expression of proteins present in multiple conditions (3 to 6 conditions) when using different isotopic labeling in a single experiment. The chromatographic system used also allows the fractionation fraction of the RH peptides to achieve the identification of a greater number of proteins.

The present invention is related with the biotechnology field, particularly with the proteomics. General speaking, we can define proteomics as a set of tools, techniques, interrelated methods for studying the proteomes. The term proteome is used to define the protein complement of the genome.

Today the combination of separation technologies, mainly two-dimensional gel electrophoresis (2DE) in combination with mass spectrometry and the automatic database search have made possible the highthroughout identification of proteins in complex mixtures. 2DE is the most resolving technique for separating complex mixture of proteins, however it has several limitations (Membrane proteins and proteomics: Un amour impossible. Santoni V., Molloy M., Rabilloud T. Electrophoresis. 21, 1054-1070, 2000; Proteome profiling-pitfall and progress. Haynes P. A., Yates Ill J. R. Yeast. 17, 81-87, 2000):
Difficulties in the analysis of very hydrophobic proteins. For example, it is well known that membrane proteins which are always attractive candidates for the vaccine development are under-represented in 2DE gels.
Proteins with extreme p/can not be focalized in an effective manner.
The obtantion of reproducible and high quality 2DE gels require high laboriousity and manual skill of the specialist.
It cannot be directly coupled to mass spectrometers so the high throughput analysis cannot be achievable in a reasonable time.
The automatic image analysis for detecting differentially expressed proteins is not efficient enough to avoid the manual analysis of an specialist and it is time consuming.

The fact that the sequencing by mass spectrometry of only 3 to 5 amino acids of a peptide is enough to carry out a reliable identification of the source protein (Error-tolerant identification of peptides in sequence databases by peptide sequence tags. Mann M, Wilm M., Anal. Chem. 1994, 66, 4390-4399) and that the proteolytic digestion of a hydrophobic protein can even generate non-hydrophobic peptides easily to be handled, makes preferable the manipulation of peptides instead of intact proteins, arising variants to carry out proteomics without using two-dimensional electrophoresis and allowing a high throughput identification of a great number of proteins in the analyzed mixtures.

The first step towards this direction was done in 1999 by Link et al. (Link, A.J. et al. Direct analysis of protein complexes using mass spectrometry. Nat. Biotechnol. 1999, 17, 676-682) when a liquid chromatography system was directly coupled to a mass spectrometer (LC-MS/MS). They packed a microcapilar column followed by a reverse-phase column. Initially, at acidic pH all peptides are retained in the cation exchanger and fractions of these peptides are eluted in batches by increasing the saline concentration of the movile phase. Then, a continuous gradient of acetonitrile, elutes the peptides retained in the reverse phase column and are analyzed by mass spectrometry in order to proceed for their identification in sequence database. This procedure is repeated several times until a complete elution of the peptides retained in the cation exchange chromatography column. This procedure known as MudPiT (Multidimensional Protein Identification Technology) has allowed the identification of 1484 proteins from the total hydrolyzed from Saccharomyces cerevisiae (Washburn M. P. et al. Large-scale analysis of the yeast proteome by multidimensional protein identification technology, Nature Biotechnology, 2001, 19, 242-247). This bidimensional fractionation is essential for identifying a great number of proteins due to the complexity of the analyzed peptide mixture and it has promoted the high throughput identification of proteins by using liquid chromatography coupled to mass spectrometry.

However, the relative quantification of proteins in complex mixtures cannot be easily achieved. The first step to solve this problem was done by Washburn MP et al. (Analysis of quantitative proteomic data generated via multidimensional protein identification technology. Anal. Chem. 2002, 74, 1650-1656) when Saccharomyces cerevisiae were grown in the normal culture medium containing nitrogen-14 (¹⁴N) and the other cells in an enriched medium containing nitrogen-15 (¹⁵N). By this procedure, all the proteins derived from one conditions are labeled with ¹⁵N while the proteins of the other condition are labeled with ¹⁴N. The proteins derived from both conditions are mixed, and proceed to protein identification through the sequencing of peptides by mass spectrometry. The determination of the relative quantities of the proteins is based on the intensity ratio of the corresponding peptides labeled with ¹⁵N/¹⁴N.

This approach based on the isotopic labeling is not always possible, and only can be performed in simple organisms such as yeast, and bacteria because of high costs of the isotopic ally enriched culture media. On the other hand, it is important to point out, that the ¹⁵N-labeling introduces certain limitations in the identification process because there is a variable mass shifts between the labeled and non-labeled peptides according the number of nitrogen atoms in their sequences.

To overcome this last limitation, other authors restricted the introduction of the isotopic labeling to certain amino acids when the organism under study is grown in a culture medium depleted in a natural amino acid but it is supplemented with its corresponding labeled amino acid.

This strategy is know as SILAC (stable isotope labeling by amino acids in cell culture) and there is a considerable number of publications using the labeling with ¹²C/¹³C y ¹H/²H in culture media supplemented with labeled and non-labeled leucine, arginine or lysine in the two compared conditions (S.E. Ong, B. Blagoev, I. Kratchmarova, D. B. Kristensen, H. Steen, A. Pandey, M. Mann, Mol. Cell Proteomics. 2002, 1, 376-386; Berger S J, Lee S W, Anderson G A, Lijiana PT, TolićN, Shen Y, Zhao R, Smith R D High-throughput Global Peptide Proteomic Analysis by Combining Stable Isotope Amino Acid Labeling and Data-Dependent Multiplexed-MS/MS. Analytical Chemistry 2002, 74, 4994-5000; and Precise peptide sequencing and protein quantification in the human proteome through in vivo lysine-Specific Mass Tagging, J. Am. Soc. Mass Spectrom. 2003, 14, 1-7). The mass shift of the light amino acid respect to the heavy amino acid, only is detected if the peptide contain the labeled amino acid, therefore peptides that do not contain the labeled amino acid cannot be used for quantification. SILAC cannot be used universally in all proteomics experiments because its high costs and it is only applicable to biological problems that are studied in cellular culture. The most universal method to perform the quantification is the ¹⁸O-labeling. It consists in the proteolytic digestion of the whole proteins from one condition in the presence of normal water (H₂O) while the proteins from the other condition are hydrolyzed in presence of ¹⁸O-labeled water (H₂¹⁸O). The peptides obtained in the buffer prepared with H₂¹⁸O can incorporate either one or two ¹⁸O at their C-terminal end, on the contrary, the other peptides show their natural isotopic ion distribution. To perform the quantification, the labeled and non-labeled peptides are mixed and the ratio of the area corresponding to the isotopic ion distribution of the species labeled with ¹⁶O/¹⁸O in the mass spectra is proportional to the relative concentration of protein that originated them in the compared samples.

This method have two limitations: (1) it does not yield a sufficient separation between the isotopic ion distributions of the labeled and non-labeled peptides and (2) the addition of ¹⁸O is not homogeneous, adding one or two ¹⁸O atoms. Both problems make difficult the relative quantification of the species unless software allows the appropriate interpretation of the complex overlapping of the isotopic ion distributions.

To overcome this problem Yao et. al (Yao X, Afonso C and Fenselau C. Dissection of proteolytic ¹⁸O-labeling: endoprotease-catalyzed ¹⁶O-to-¹⁸O exchange of truncated peptide substrates. J. Proteome Res. 2003, 2, 147-52) proposed the complete labeling of peptides with ¹⁸O after the proteolytic digestion by a extended incubation of the peptides in the presence of trypsin in order to guaranteed the complete incorporation of two ¹⁸O atoms at the C-terminal end of the peptides and thus exists a separation of 4 Da between the isotopic ion distribution of the labeled and non-labeled peptides.

However, on one hand, there are peptides that are resistant to the additional incorporation of ¹⁸O once they were generated by the cleavage of trypsin an introduce considerable errors in the quantifications, and, on the other hand, the additional incubation for long times to ensure the complete exchange of ¹⁶O by ¹⁸O may introduce non-specific cleavages in the peptide sequence and it affect the protein identification in the sequence database.

Karen et al. implemented a new method named inverse ¹⁸O-labeling (Y.Karen Wang, Zhixian M a, Douglas F. Quin, W . Fu. Inverse ¹⁸O Labeling Mass Spectrometry for the Rapid Identification of Marker/Target Proteins. Anal. Chem. 2001, 73, 3742-3750) where to pool of proteins (composed of eight proteins), the control sample and the test sample were prepared. Each pool of proteins is divided in two equal fractions and digested in different buffers prepared with ¹⁸O and ¹⁶O. After finishing the digestions, they make two pools of mixtures, where each group were represented by the fractions labeled with the different isotopes. This original method allows the simplification in the data analysis because the substraction between both experiments eliminate all the proteins that did not changed their expression level; allow the detection of modification originated due to certain perturbations, is suitable for the high throughput analysis; and simplify the analysis because it did not introduce any restriction to the signal that changed their expression level.

The inverse labeling facilitates the identification of proteins with extreme differential expression, however this method require two experiments and it limitates its applicability because it require higher amount of sample and it is very often a limiting factor in proteomics.

The ¹⁸O-labeling also has been used in quantitative proteomics for the specific ¹⁸O-labeling of N-glycopeptides when one of the samples is deglycosylated in a buffer prepared with H₂¹⁸O and the other in a buffer prepared with normal water (Kuster, B and Mann M. ¹⁸O-labeling of N-glycosylation sites to improve the identification of gel-separated glycoprotein's using peptide mass mapping and database searching. Anal. Chem. 1999, 71, 1431-1440). After mixing equal proportion of the analyzed samples, the relative quantities of the individual proteins are estimated in the same way, by the ratio ¹⁶O/¹⁸O present at the sidechain of Asp obtained in the deglycosylation reaction of the N-glycopeptides (Gonzalez J, Takao T, Hori H, Besada V, Rodriguez R, Padron G, Shimonishi Y. A Method for Determination of N-Glycosylation Sites in Glycoprotein's by Collision-Induced Dissociation Analysis in Fast Atom Bombardment Mass Spectrometry: Identification of the Positions of Carbohydrate-Linked Asparagine in Recombinant α-Amylase by treatment with PNGase-F in ¹⁸O-labeled Water. Anal. Biochem., 1992, 205, 151-158).

In fact, the high specificity of this method for the N-glycopeptides has been proposed as a strategy to discriminate false-positive identifications (Kuster, B and Mann M. ¹⁸O-labeling of N-glycosylation sites to improve the identification of gel-separated glycoprotein's using peptide mass mapping and database searching. Anal. Chem. 1999, 71, 1431-1440) by restricting the database search to peptides containing potential N-glycosylation sites (Asn-X-Ser/Thr). Another variant to perform a quantitative proteomics was introduced by Zappacosta and Annan (Zappacosta F, and Annan RS. N-terminal isotope tagging strategy for quantitative proteomics: results-driven analysis of protein abundance changes. Anal Chem. 2004, 76, 6618-6627) and it comprises the introduction of the labeling in all proteolytic peptides after transforming all the lysine residues into homoarginines and derivatize all the amino terminal groups of proteolytic peptides obtained in one of the compared conditions with a blocking reagent enriched with heavy isotope (particularly deuterium) while the amino terminal groups of the peptides obtained in the other condition are modified with the non-labeled reagent. After mixing both samples, the quantification is performed after estimating the intensity ratio of the isotopic distribution corresponding to the light- and heavy-peptide.

To avoid the overlapping in the isotopic distribution of the labeled and non-labeled species, the derivatization of peptides is performed with deuterated (d₅) and normal propionic anhydride. However, the incorporation of more than 3 atoms of deuterium might introduce errors in the relative quantification of the light and heavy species because of their different retention times in the reversed phase chromatography as demonstrated by Zhang and coworkers (Zhang R, Sioma C S, Wang S, Regnier F E. Fractionation of isotopic ally labeled peptides in quantitative proteomics. Anal Chem. 2001 73: 5142-5149). The errors in the quantification may increase in the same way increase the number of deuterium atoms incorporated in the amino acid sequence of the heavy specie (Zhang R, Sioma C S, Thompson R A, Xiong L, Regnier F E. Controlling deuterium isotope effects in comparative proteomics. Anal Chem. 2002; 74, 3662-3669) and it has been reported that these errors are minimized when the blocking is performed with the ¹³C-labeled reagents (Zhang R, Regnier F E, Minimizing resolution of isotopic ally coded peptides in comparative proteomics. J. Proteome Res. 2002, 1,139-147).

The analysis of the proteolysis of complex protein mixtures constitutes a great challenge since the overwhelming number of peptides that are generated, surpasses the resolution power of the current chromatographic systems and the most modern mass spectrometers as well.

To approach this challenge and to carry out quantitative proteomics without the necessity of the usage of the two-dimensional electrophoresis, a trend has been the simplification of the peptide mixture by the development of methods that allow the selective isolation of a subset of peptides that possess a common characteristic, and that its study doesn't affect the representativeness of the original proteins, allowing the characterization the biggest number of proteins present in the initial mixture. The combination of a selective isolation of peptides with appropriate isotope labeling techniques not only allows the identification but also the relative quantification of the proteins present in the compared initial mixtures.

Selective Isolation of Cysteine Containing Peptides

This approach was initiated by Gigy and coworkers (Quantitative analysis of complex protein mixes using isotope-coded affinity tags. Gygi, S. P., Rist, B., Gerber, S. A., Turecek, F., Gelb, M. H., and Aebersold, R. Nat. Biotechnol. 17, 994-999, 1999) when proposing the well-known ICAT method (isotope-code affinity tags) that is based in the selective isolation of cystein-containing peptides. The method combines the affinity chromatography (streptavidin-biotin) and the labeling with the ICAT reagent in its light- and heavy-variants. This reagent consists of three functional elements:
1—a group that reacts specifically with the thiol groups of the cystein residues.
2—a group that has an affinity element (biotin) and allows the selective isolation of the peptides that react with ICAT.
3—an arm that separates the two elements before mentioned, in the heavy variant it has 8 deuterium atoms in its structure (heavy ICAT) and the light variant has 8 atoms of hydrogen (light ICAT).

Once the proteins from both conditions are separately reduced in presence of DTT, the free cysteines generated in one of the conditions react with the heavy-ICAT and those generated in the other condition with the light-ICAT. Both mixture of proteins are joined in identical quantities and proceeds the proteolytic digestion. The generated peptides are purified by a streptavidine affinity column and as a consequence, the cystein containing peptides modified with the ICAT reagent are selectively isolated. To proceed to the relative quantification, the relative intensities of the signals corresponding to the peptides labeled with the light and heavy ICAT are measured. The masses of the peptides labeled with these reagents differ in multiples of 8 units of masses depending the number of cysteine residues contained in the sequence.

This methodology presents several limitations:
The size of the ICAT reagent is considerable and it can affect the ionization efficiency of peptides and the interpretation of the mass spectra.
The presence of 8 deuterium atoms in the peptide modified with the heavy ICAT can cause big errors in the quantification because its retention time can differ considerably respect to the peptide modified with the light ICAT (Zhang R, Sioma C S, Wang S, Regnier F. Fractionation of isotopic ally labeled peptides in quantitative proteomics. Anal Chem. 2001, 73, 5142-5149) and the intensities ratios are not the same during the elution time of the light and heavy species of the peptide to be quantified.
The quantification procedure described for the ICAT, is not applicable to other methods of isotopic labeling that do not separate enough the signals to avoid the overlapping of its isotopic distributions.
If one wants to fractionate the peptide mixture additionally in a similar way as the MudPit, (Washburn M. P. et al. Large-scale analysis of the yeast proteome by multidimensional protein identification technology. Nature Biotechnol. 2001, 19, 242-247) it is necessary to use a chromatography different to the used for the selective isolation of peptides and it causes considerable losses in the manipulation of the samples.
When using a high affinity chromatography during the selective isolation step, the losses can be considerable for some particular peptides.
Proteins without cysteines cannot be analyzed by this method.
   Selective Isolation of Methionine Containing Peptides.

Another method has been implemented for the selective isolation of methionine-containing peptides by using a combined diagonal chromatography and it is known in the literature as the COFRADIC technology (Gevaert K, Van Damme J, Goethals M, Thomas G R, Hoorelbeke B, Demol H, Martens L, Puype M, Staes E, Vandekerckhove J.

Chromatographic isolation of methionine-containing peptides for gel-free proteome analysis: identification of more than 800 Escherichia coli proteins. Mol Cell Proteomics. 2002, 11, 896-903). After the reduction and S-alkylation, all proteins are digested and the peptides are fractionated by reverse phase chromatography and collected in a considerable number of fractions in what the authors denominate as a primary run. Each one of these fractions reacts with a solution of hydrogen peroxide (3%) during 3 minutes, and they are newly analyzed in the same chromatography system under the identical conditions in a second run. The methionine-containing peptides once oxidyzed are isolated selectively because they are transformed in less hydrophobic species, diminish their retention time, so they differ of the rest of the peptides that do not contain methionine whose retention times remain invariable in the second run and they are discarded. According to the authors this method allows a simplification of the complex mixture of peptides in a similar extension to the one obtained by the ICAT and it can be applied to the selective isolation of phosphopeptides, N-terminal peptides of proteins and peptides linked by disulfide bridges (Martens L, Van Damme P, Damme JV, Staes E, Timmerman A, Ghesquiere B, Thomas GR, Vandekerckhove J, Gevaert K. The human platelet proteome mapped by peptide-centric proteomics: To functional protein profile. Proteomics. 2005, 5(12), 3193-3204).

Although the authors outline that the conditions of the oxidation have been optimized to avoid the modification of labile residues like cysteines and tryptophans, if this happened the selectivity of the method would be affected and the simplification degree of the peptide mixture that would be reached would not be similar to that of the ICAT method as the authors claims.

On the other hand, although it is outlined that this method is amenable to be automated, to achieve the selective isolation of all the methionine containing peptides it is necessary to carry out a great number of chromatographic runs and the global yield of the method can it turns be affected.

Selective Isolation of the N-terminal Peptides.

A variant of the COFRADIC has also been proposed to isolate selectively the N-terminal peptide of all the proteins (Gevaert K, Goethals M, Martens L, Van Damme J, Staes T O, Thomas G R, Vandekerckhove J. Exploring proteomes and analyzing protein processing by mass spectrometric identification of sorted N-terminal peptides. Nat Biotechnol. 2003; 21, 566-569). The first step of this method consists the blockage of all primary amino groups of the proteins present in the compared complex mixtures, then a specific proteolysis of the modified proteins is performed and by reverse phase chromatography, the peptide mixture is separated in a considerable number of fractions.

The new amino groups of the internal peptides generated during the proteolysis that are present in each one of these fractions react additionally with a highly hydrophobic blocking group and again are separated in the same chromatographic system under conditions identical to that of previously mentioned. The retention time of all internal peptides is increased considerably by the additions of the second blocking reagent, however all the N-terminal end peptides of proteins that were blocked in the first step are selectively isolated when being collected in the same retention time of its original fraction. This strategy have as a disadvantage: to perform a reliable quantification the first step consisting in the blocking of the aminos groups should work in a quantitative way and it is something difficult to achieve when proteins are present in complex mixtures.

Also in this first step during the blocking of amino groups the solubility of the proteins can be diminished considerably to originate precipitations that can affect in turns the quantitativity of the method. Lastly, the fact that a single peptide per protein is isolated is an excessive simplification and it may have a negative impact in the identification and the quantification of the present proteins in the complex mixtures. A method that allows redundancy by isolating a reduced group of 3-4 péptide for proteins can be ideal for proteomics studies without the usage of the two-dimensional electrophoresis since it permits the confirmation of the quantification results by other peptides of the same protein.

Selective Isolation of N-glycopeptides.

It is reported that approximately the 91 percent of the membrane proteins present in the swissprot databases are glycoprotein's (Gahmberg C G, Tolvanen M. Why mammalian cell surface proteins plows glycoprotein's. Trends Biochem. Sci. 1996, 21, 308-311). It has proposed an strategy of work based on the selective isolation of glycopeptides for proteome studies by using the lectin affinity chromatography (Geng M, Zhang X, Bina M, Regnier F. Proteomics of glycoprotein's based on affinity selection of glycopeptides from tryptic digests. J Chromatogr B Biomed Sci Appl. 2001, 752, 293-306; Kaji H, Saito H, Yamauchi AND, Shinkawa T, Taoka M, Hirabayashi J, Kasai K, Takahashi N, Isobe T. Lectin affinity captures, isotope-coded tagging and mass spectrometry to identify N-linked glycoprotein's. Nat. Biotechnol. 2003, 21, 667-672).

The usage of lectins, either a particular lectin or a battery of them immobilized in a chromatographic column, possesses a limitation since it is not able to recognize all the glycoforms present in the sample and to guarantee an efficient selective isolation. To overcome this limitation Zhang and collaborators (Zhang H, Li XJ, Martin DB, Aebersold R. Identification and quantification of N-linked glycoprotein's using hydrazide chemistry, stable isotope labeling and mass spectrometry. Nat Biotechnol. 2003, 21, 627-629) proposed the immobilization of the glycopeptides to a solid support by using the derivatization with hydrazine and then its releasing by the action of the PNGase-F. To carry out this last step in the presence and absence of H₂¹⁸O allows to proceed to the quantification.

This method can be applied to samples of biological interest such as membrane proteins where vaccine and receptor candidates are included. Also it can be applicable to the serum which is however the most complex proteome, however its applicability is restricted to those samples that are enriched in glycoprotein's.

Selective Isolation of Histidine-containing Peptides.

The immobilized chelate metal affinity chromatography has been used in proteomics for the selective isolation of peptides that contain histidine. There are several works that evaluate different matrixes and the immobilized metals ions (Ren D, Penner N A, Slentz B E, lnerowicz H D, Rybalko M, Regnier F E. Contributions of commercial sorbents to the selectivity in immobilized metal affinity chromatography with Cu(II). J Chromatogr A. 2004 1031, 87-92) but the results demonstrate that the specificity is still inferior compared with that of the other previously described methods for the selective isolation of peptides (Ren D, Penner N A, Slentz B E, Mirzaei H, Regnier F. Evaluating immobilized metal affinity chromatography for the selection of histidine-containing peptides in comparative proteomics. J Proteome Res. 2003, 2, 321-329; Ren D, Penner N A, Slentz B E, Regnier F E. Histidine-rich peptide selection and quantification in targeted proteomics. J Proteome Res. 2004, 3, 37-45). In fact, variants of chemical modification of peptides before the affinity chromatography have been explored for increasing the specificity.

Selective Isolation of Peptides by Cation Exchange Chromatography.

The cation exchange chromatography has been used for the selective isolation of peptides by separating in an easy manner the neutral peptides (zero charge) from the positively-charged peptides (chage: 1+, 2+, 3+, 4+, etc). Initially it was used to isolate the C-terminal peptide (A new method for the isolation of the C-terminal peptide of proteins by the combination of selective blocking of proteolytic peptides and cation-exchange chromatography. Chapter #12 in: Proteome and Proteome Analysis. (1999) Springer Verlag publishers); and (Isolation and characterization of modified species of a mutated (Cysl¹²⁵-Ala) recombinant human interleukin-2. Moya G, Gonzalez L J, Huerta V, Garcia Y. J. Chromatogr. 2002, 971, 129-142) and blocked N-terminal peptides (Selective isolation and identification of N-terminal blocked peptides from tryptic digests. Betancourt L, Besada V, Gonzalez L J, Morera V, Padron G, Takao T, Shimonishi Y. J. Pept. 2001, 7, 229-237). However, these methods cannot be applied to the massive study of complex mixtures of proteins and proteomes because a single peptide per protein is isolated and for a more reliable quantification of the identified proteins is required the isolation of a bigger number of peptide per proteins, (approximately 3-4 peptides/proteins). On the other hand, if the identification of the proteins is based on a single peptide per protein there is a considerable risk of missing the identification of a high number of proteins since all the peptides do not fragment efficiently in the spectrometer of masses and it is not always possible to obtain structural information. In these two methods of selective isolation, the peptides are obtained as neutral species (chage zero) and they do not have any basic amino acids at their C-terminal end and it does not facilitate the fragmentation in the spectrometer and consequently its identification in the sequence database.

The method for the selective isolation of peptides based on the cation exchange chromatography was also applied in proteomics studies to selectively isolate peptides that do not contain neither histidine nor arginine, denominated by the authors as nHnR peptides (SCAPE: A new tool for the Selective Captures of Peptides in Protein identification. Betancourt L, Gil J, Besada V, Gonzalez L J, Fernández-of-Cossio J, García L, Pajón R, Sánchez A, Alvarez F, Census G. J. Proteome Res. 2005, 4, 491-496). This method achieved a considerable simplification of the analyzed peptide mixture in a similar extension to the one achieved by the ICAT (Quantitative analysis of complex protein mixes using isotope-coded affinity tags. Gygi, S. P., Rist, B., Gerber, S. A., Turecek, F., Gelb, M. H., and Aebersold, R. Nat. Biotechnol. 17, 994-999, 1999). The chromatographic system used in this method also separates charged peptides (most of the part composed by peptides that contain arginine and histidine) from neutral composed mainly by the nHnR peptides, completely blocked at their primary amino groups. Before analyzing the nHnR in the mass spectrometer, the attached blocking group is eliminated by means of a hydrolytic treatment to regenerate the free amino groups and to make more favorable and efficient its ionization, fragmentation and consequently its identification in the databases.

This method isolates the nHnR peptides in the non-retained fraction of the cation exchange chromatography and to achieve the identification of a higher number of proteins it requires another chromatographic step for its additional fractionation. These additional chromatographic steps may cause losses during the manipulation and affect the yields of the method.

The discarded fraction in this method is enriched in peptides that possess arginine and histidine. The arginine presumably should be located in the C-terminal end of the peptides because they were generated by the cleavage of trypsin and the histidine residues should be located inside the sequence.

The triptic peptides with these characteristics fragments very well in mass spectrometry because in the ionization process they possess a mobile proton (Paizs B. Suhai S. Towards understanding the tandem mass spectra of protonated oligopeptides: mechanism of amide bond cleavage. J Am Soc Mass Spectrom. 2004 15,103-13; Cordero M M, Houser J J, Wesdemiotis C. The neutral products formed during backbone fragmentations of protonated peptides in tandem mass spectrometry. Anal. Chem. 1993; 65,1594-1601) within their sequence (located at the histidine) and it can induce multiple fragmentations along the polypeptide chain, having at the same time a fixed proton located at the end C-terminal end of the peptide (located at the arginine). Their ESI-MSMS spectra produced by the collision induced dissociation dissociation experiments is abundant of Y\ddot{}n series with enough structural information to achieve a highly reliable identification in the databases.

The analysis of peptides with these characteristics is attractive, however, the fraction that is discarded in the SCAPE method is still very complex. The tryptic digest of a complex mixture of proteins of a given proteome generates approximately an average of 18-20 peptides/proteins and if the SCAPE simplifies this complex mixture when isolating selectively 4-5 peptides/protein it implies that the discarded fraction (mentioned above) should have an average of 14-15 peptides/proteins approximately. This last value is excessively high and it is not the optimum to achieve a proteomic study without using two-dimensional electrophoresis and a further simplification of this fraction is still necessary. Therefore, the development of a new 2DE-free method based on the selective isolation of peptides enriched in basic residues: arginine and histidine is required.

In spite of the limitations described for these methods it continues being very necessary to identify and to determine expression levels of the present proteins in complex mixtures, through the selective and specific isolation of a small group of peptides by means of the simplification of the complex mixture to be characterized by mass spectrometry.

### DETAILED DESCRIPTION OF THE INVENTION

The method for the selective isolation of RH peptides is achieved by combining the blocking of the primary amino groups of proteolytic peptides and a step of cation exchange chromatography at acidic pH. This combination simplifies the complex mixture of peptides by eliminating in an effective and simple way a majority subset of all proteolytic peptides that possess only one or less arginine or hisitidine residues within their sequences (R+H≦1) and it analyzes only a small subset of peptides composed by those that are retained selectively in the cation exchange column and possess multiple positive charges, the peptides that possess in their sequences more than one basic residue arginine or hisitidine (R+H>1).

This method can be used for the identification of the proteins constituent of complex mixtures and for the determination of their relative quantities under the compared conditions. For this purpose, the mixture of proteins obtained either by artificial or natural way should be treated according to the steps that are described in the FIG. 1 and we explain below:
(1) Reduction and S-alkylation of cyteine residues with any of the reagents used for this purpose, for example iodoacetamide, iodoacetic acid, acrylamide, 4-vinylpiridine, etc. This initial step is of a great importance due to several reasons: (a) it assures a bigger efficiency of the next step of the enzymatic hydrolysis of the peptide bonds of proteins present in the analyzed mixture; (b) it avoids the cross-linking of peptides of different proteins that possess cystein residues; (c) it facilitates the identification of the proteins in the databases.
(2) Hidrolysis of proteins. This step can be achieved through an enzymatic digestion using trypsin, or other endoproteinases such as Glu-C, Asp-N, Lys-C, chymotrypsin, termolisin, pepsin, papain, pronase or other proteasas; a chemical hydrolysis can also be used with cyanogen bromide, with organic or inorganic acids. The hydrolysis of the peptide bonds can be carried out by means of the combination of the enzymatic and/or chemical procedures previously described. The peptides generated by these treatments possess an appropriate size for their mass spectrometry identification.
(3) Blocking Reaction consists on the covalent modification of the amino terminal groups and epsilon-amino groups of the lysine residues that contains all the peptides generated by some of the treatments described previously. In this method a wide range of blocking reagents of the amino groups can be used whenever these they do not provide net positive charges to the structure of the modified peptides or they do not possess groups that can be protonated at acidic pH and contribute to the positive charge of the peptide. Among the blocking reagents are: acetic anhydride, N-hydroxysuccinimide, N-acetoxysuccinimide, citacronic anhydride, maleic anhydride, succinic anhydride, ftalic anhydride, tetrahydroftalic anhydride, 9-fluorenylmethyl chloroformate (Fmoc-Cl), 2-methyl sulfonyl ethyl succinimidyl carbonate), urea and reagents that provides protecting amino groups such as: (a) aromatic urethane-type protecting groups which include benzyloxycarbonyl, 2-chlorobenzyloxycarbonyl, 9-fluorenylmethyloxycarbonyl, isonicotinyloxycarbonyl and 4-methoxybenzyloxycarbonyl; (b) aliphatic urethane-type protecting groups which include t-butoxycarbonyl, t -amyloxycarbonyl, isopropyloxycarbonyl, 2-(4-biphenyl)-2-propyloxycarbonyl, allyloxycarbonyl and methylsulfonylethoxycarbonyl; (c) cycloalkyl urethane -type protecting groups which include adamantyloxycarbonyl, cyclopentyloxycarbonyl, cyclohexyloxycarbonyl and isobornyloxycarbonyl; (d) acyl protecting groups or sulfonyl protecting groups. Preferred protecting groups include benzyloxycarbonyl, t-butoxycarbonyl, acetyl, 2-propylpentanoyl, 4-methylpentanoyl, t-butylacetyl, 3-cyclohexylpropionyl, n -butanesulfonyl, benzylsulfonyl, 4-methylbenzenesulfonyl, 2-naphthalenesulfonyl, 3-naphthalenesulfonyl and 1-camphorsulfonyl; (e) photosensitive protective groups which include carbamates derivatives from m-nitrophenyl, 3,5-dimetoxybenzyl, 1-methyl-1 (3,5-dimetoxyphenyl)etyl, α-methylnitropiperonyl, o-nitrobenzyl, 3,4-dimetoxy-6-nitrobenzyl, phenyl(o -nitrophenyl)methyl, 2-(2-nitrophenyl)etyl, 6-nitroveratryl, 4-metoxyfenacyl and 3′,5′-dimetoxybenzoine

In general, all the reagents used in the peptide synthesis for the protection of amino groups or other reagents can also be used if they fulfills the previously explained properties. Examples of this type of reagents and the protocols to carry out the modification of amino groups are easily find in the scientific literature (Protective groups in organic synthesis, Teodora W. Greene and Peter G. M. Wuts, p. 494-654, E d. John Wiley & Sons, Inc. (1990) and Peptide Chemistry, Bodanszky, N., p. 74-103, Springer-Verlag, New York (1988) and their use is included in the present invention. This blocking reaction of the primary amino groups (alpha-amino terminal and epsilon-amino of lysines) is very important, so that the positive charge of the peptides at acidic pH only depends on the number of two other basic amino acids present in the peptide sequences: arginine (R) and histidine (H).
(4) Cation exchange chromatography of the mixture of modified peptides. The selective isolation of peptides is performed by using a strong cation exchanger at acidic pH (for example a pH between 2 and 4) containing acetonitrile (10 % v/v) in the equilibrium solution. To obtain this pH range, can be used formic, acetic, trifluoroacetic, and heptafluorobutiric acids or other buffers systems or solvents that provides the required pH. This chromatographic step separates in an effective way and with a great selectivity the peptides in two groups: (a) neutral peptides or having a single positive charge (charge 0 and 1+) and (b) multiply-charged peptides (charge 2+, 3+, 4+, etc). For neutral or monocharged peptides it understands those where the sum of the number of arginine residues (R) or hisitidine (H) in their respective sequences is inferior or equal to 1 (R+H ≦1). These peptides are not analyzed and they are discarded in the non-retained fraction by the cation exchange column. The multiply-charged ipeptides are those that possess more than one arginine residues and/or histidine within their sequence (R+H>1), they are denominated in the present invention as RH peptides and their selective isolation represents a considerable simplification of the mixture of analyzed peptides without sacrificing considerably the representativeness of the origin proteins (see FIG. 1).

The chromatographic system developed here possesses a double function since it not only allows the simplification of the analyzed complex mixture when the RH peptides are selectively isolated but it also allows its further fractionation by applying a gradient increasing the ionic strength or the pH of the mobile phase before being analyzed by reverse phase chromatography coupled to the mass spectrometer. This additional fractionation by cation exchange chromatography is key for the identification of a great number of proteins (Washburn M. P. et al. Large-scale analysis of the yeast proteome by multidimensional protein identification technology, Nature Biotechnology 2001, 19, 242-247). The method of this invention uses desalting steps in the reverse phase columns before the cation exchange chromatography to eliminate the excess of reagents or to change the working solutions.
(5) Elimination of side reactions. Before being analyzed in the mass spectrometer, the peptides undergo a basic or acidic treatment in presence of a base or acid of organic or inorganic origin to a concentration from 1 to 5% to revert the side reactions in other amino acids (such as tyrosine, serine or threonine) that could have been produced during the blocking reaction. This step can be used additionally to eliminate the blocking group introduced in the step (3) in case of a reversible blocking is used and it is wanted that RH peptides does not have any blocking group at their amino groups before the analysis by mass spectrometry.

To apply this method in the quantitative proteomics it is necessary that the generated peptides in one of the two compared conditions carry one or several heavy isotopes (¹³C, ¹⁵N, ¹⁸O, and/or ²H) in their structure while the peptides generated in the other condition possesses the same elements previously mentioned but with their natural isotopic abundances (¹²C, ¹⁴N, ¹⁶O, and/or ¹H). The elimination of the blocking group of the amino groups of the RH peptides only would be made when this it does not carry the isotopic labeling that is essential to achieve the relative quantification. If the modifier reagent is of a photosensible nature, this can be eliminated by light irradiation of the modified peptides.

The incorporation of the heavy isotopes in the structure of the generated peptides during the proteolysis of proteins in one of the two compared conditions can be carried out in three different ways:
(a) Isotopic labeling introduced by the cell. It is applied to protein extracts from tissues or cells cultivated in media with two isotopic variants of certain essential nutrients for the growing of the cells. The biosintetic machinery incorporates these isotopes in certain amino acids or in all those amino acids that possess the used isotope. Among the used nutrients, labeled compounds that constitute nitrogen source (¹⁴N/¹⁵N); essential amino acids for the cells, labeled in certain positions with isotopes of hydrogen (¹H/²H), nitrogen (¹⁴N/¹⁵N), carbon (¹²C/¹³C), oxygen (¹⁶O/¹⁸O), sulfur, etc. Later on, both extracts of total proteins are mixed, hydrolyzed and the method is continued according to that described in the steps from the 1 to the 5 to achieve the selective isolation of the RH peptides
(b) Isotope labeling introduced during the proteolysis. The protein samples to be compared are hydrolyzed independently, as described in the step 2 of the method, in one case in water enriched with ¹⁸O (H₂¹⁸O) and in the other case, with water that possesses their natural isotopic abundance. All poteolytic peptides derived from the first condition are labeled when incorporating one or two atoms of ¹⁸O at their carboxy C-terminal end. Later on, before mixing equal quantities of the digested proteins in both conditions, the proteolytic activity of the enzyme is inhibited by adding of a cocktail of protease inhibitors, or specific inhibitors of the used proteases. The method proceed in the way indicated in the steps 3-5. When this isotopic labeling is used it is advisable a removal of the blocking groups at basic pH because the prolonged treatments of the ¹⁸O-labeled peptides at acidic pH lead to the partial or total loss of the labeling introduced by enzymatic treatment at the carboxy C-terminal end of the peptides and it introduces a great errors in the quantification. This variant of isotopic labeling is frequently used when the heavy isotopes are not introduced at the N-terminal end blocking groups, in other words, when the blocking reagent is just used to modify the primary amino groups of the peptides generated in the compared conditions but not for introducing the labelig responsible for the quantification.
(c) Isotopic labeling introduced during the blocking reaction. The mixtures of proteins to be compare are digested with a protease separately according to the step 2 of the proposed method and the primary amino groups of the obtained peptides are modified using isotopic variants of the blocking reagent. The blocking group of the primary amino groups possesses two variants, given by the presence of light or heavy isotopes to allow the quantification. For example when using the following reagents (C²H₃CO)₂O/(C¹H₃CO)₂O or (¹³CH₃CO)₂O/(¹²CH₃CO)₂O or (¹³CH₃¹³CO)²O/(¹²CH₃¹²CO)²O, the peptides are labeled with ²H₃/¹H₃ or ¹³C₁/¹²C₁ or ¹³C₂/¹²C₂, respectively. This principle is also valid for every of the blocking reagents mentioned in the step (3) of the described method that carry anyone of the isotopes above mentioned as well as ¹⁸O/¹⁶O, ¹⁵N/¹⁴N, ³⁵S/³²S, etc. After labeling, the RH peptides are isolated by cation exchange chromatography as explained in the steps of the described method. In this type labeling the transitory or reversible blocking of the amino groups cannot be used because if it is eliminated before the mass spectrometric analysis, the isotopic labeling is also eliminated and therefore the relative quantification is impossible to be carried out.

The relative quantification by the analysis of the isotopic distribution of the mixture of the labeled and non-labeled RH peptides in the mass spectra is carried out by using an appropriate software (Fernández of Cossio et al. Isotopic, A Web Software for Isotopic Distribution Analysis of Biopolymers by Mass Spectrometry. Nuclei Acid Research 2004, 32, W674-W678 and Fernández of Cossio et al. Automated Interpretation of Mass Spectra of Complex Mixtures by Matching of Isotope Peak Distributions. Rapid Commun. Mass Spectrom. 2004; 18, 2465-2472). This software calculates the theoretical isotopic distributions of the labeled and non-labeled RH peptides and it achieves a such combination so that the resultant area of the theoretical isotopic distribution is adjusted from the best way to the area of the isotopic distribution observed experimentally. The proportions existent between each area corresponding to the peptides labeled with light and heavy isotopes (¹²C/¹³C, ¹⁴N/¹⁵N, ¹⁶O/¹⁸O, and/or ¹H/²H) once normalized correspond with the relative proportion of the proteins that contained them in the compared mixtures.

To carry out the quantification it is necessary to know (a) the elemental composition of the analyzed peptide or its sequence, (b) the type of isotopic labeling used in the experiment and (c) the region of the mass spectrum that contains the experimental isotopic distribution of the RH peptide of interest. All this information is very restrictive allowing to calculate in a very precise way the experimental noise and to discard of the analysis the overlapping of other signals that are not of interest for the quantification. All these information's make the quantification process very robust with the used software and it is independent of the method of isotopic labeling. This proposed method is compatible with the ionization modes more frequently used in the characterization of peptides and proteins: the electrospray ionization for (ESI-MS) and the matrix assisted laser desorption ionizaton (MALDI-MS). These ionization methods give the molecular mass, an important information, however they do not provide structural information that allows a reliable identification in the sequence databases of the selectively isolated RH peptides. To achieve this, the peptides of interest are selected in the mass spectrometer and pass through a collision chamber where by means of a process known as collision induced dissociation, fragments containing enough structural information are generated and it allows the elucidation of the complete or partial sequence of the analyzed peptide.

The mass spectrum that contains this information is known as MSMS spectrum. Each MSMS spectrum is very characteristic of the sequence of the peptide that originated it, can be considered as a fingerprint of the fragments ions and it is useful for the reliable identification of the peptides in the sequence databases with the aid of computer programs. In fact this it is the principle of one of the most popular search engines designs for the identification of proteins in the sequence databases: the program MASCOT (Matrix Science Ltd, U K, Perkins, D N, et al. Probability-based protein identification by searching sequence databases using mass spectrometry data. Electrophoresis 1999, 20, 3551-3567); and the SEQUEST program (Trademark, University of Washington, Seattle Wash., McCormack, A. L. et al. Direct Analysis and Identification of Proteins Mixtures by LC/MS/MS and Database Searching at the Low-Femtomole Level. Anal. Chem. 1996, 69, 767-776; Eng, J. K. et al. An Approach to Correlate Tandem Mass Spectral Dates of Peptides with Amino Acid Sequences in to Protein Database. J. Amer. Soc. Mass. Spectrom. 1994, 5, 976-989; U.S. Pat. No. 5, 538,897 (Jul. 23,1996) Yates, Ill et al.)

These programs (MASCOT and SEQUEST) compare the MSMS spectra obtained experimentally with the theoretical MSMS spectra of all the peptides that possess a certain molecular mass in the protein sequences databases and they have been originated by the specific cleavage of the used protease. The MSMS spectrum where a bigger coincidence exists between the mass values of the theoretical fragments and those obtained experimentally, should correspond with the analyzed peptide and from here is inferred the protein that containing it and the identification in the database is carry out.

The following references are related with the application of some mass spectrometry techniques to the identification of proteins, particularly in the proteome analysis: Ideker T, Thorsson V, Ranish J A, Christmas R, Buhler J, Eng J K, Bungarner R, Goodlett D R, Aebersold R, Hood L. Integrated genomic and proteomic analyses of to systematically perturbed metabolic network. Science. 2001, 292, 929-934; Gygi S P, Aebersold R. Mass spectrometry and proteomics. Curr Opin Chem Biol. 2000, 4, 489-494., Gygi S P, Rist B, Aebersold R. Measuring gene expression by quantitative proteome analysis. Curr Opin Biotechnol. 2000, 1, 396-401, Goodlett D R, Bruce J, Anderson G A, Rist B, Pass-Tolic L, Fiehn O R, Smith R D, Aebersold R. Protein identification with to it sails accurate mass of to cysteine-containing peptide and constrained database searching. Anal Chem. 2000, 15; 72, 112-118; and Goodlett D R, Aebersold R, Watts J D. Quantitative in vitro kinase reaction as a guide for phosphoprotein analysis by mass spectrometry. Rapid Commun Mass Spectrom. 2000; 14, 344-348).

Taking into account the high selectivity of the proposed method, the identification can be restricted to databases that only possess RH peptides to guarantee a faster identification, to avoid false positive identification and to obtain a more reliable identification by using the programs MASCOT and SEQUEST.

### DESCRIPTION OF FIGURES

FIG. 1. Diagram that shows the selective isolation of RH peptides using the method described in this invention for their application in the quantitative proteomics.

FIG. 2. The selective isolation of the RH peptides is shown in a model protein: the recombinant streptokinase (rSK). (A) ESI-MS spectrum of the mixture of tryptic peptides. (B) ESI-MS spectrum of the peptide mixture after the blocking reaction of the amino groups. (C) ESI-MS spectrum that contains the RH peptides of the rSK after applying the method object of this invention.

FIG. 3. Results provided by the MASCOT program where the three proteins (rSK, myoglobin and cytochrome-C) present in the artificial mixture are automatically identified after the selective isolation of the RH peptides and the analysis by LC-MS/MS.

FIG. 4. The ESI-MS spectra in black color shows the isotopic distributions of the peptides ²²¹DSSIVTHDNDIFR²³³ (rSK), ²⁸TGPNLHGLFGRK³⁹ (cytochrome-C) and ⁷⁹KGHHEAELKPLAQSHATK⁹⁶ (myoglobin) obtained after applying the method for selective isolation shown in the example 1 to an artificial mixture of three proteins (rSK, myoglobin and cytochrome-C) and using the irreversible blocking of the amino groups with normal acetic anhydride [(CH₃CO)₂O] and deuterated acetic anhydride [(C²H₃CO)₂O] see experiment 1, example 3). In blue and violet colors are shown the contours of the isotopic distributions corresponding to the above mentioned peptides modified with a normal acetyl group (CH₃CO)—and a deuterium labeled acetyl group (C²H₃CO)—, respectively. The contour of the isotopic distribution highlighted in red color show the adjustment carried out by the program after superimposing the isotopic distributions mentioned previously in the proportion (CH₃CO/C²H₃CO) determined by the software as it is indicated for each particular peptide.

FIG. 5. The ESI-MS spectra in black color shows the isotopic distributions of the peptides ²²¹DSSIVTHDNDIFR²³³ (rSK), ²⁸TGPNLHGLFGRK³⁹ (cytochrome-C) and ⁷⁹KGHHEAELKPLAQSHATK⁹⁶ (myoglobin) obtained after applying the method of selective isolation shown in the example 1 to an artificial mixture of three proteins (rSK, myoglobin and cytochrome-C) and using the irreversible blocking of the amino groups with normal acetic anhydride .[(¹²CH₃CO)₂O] and ¹³C-labeled acetic anhydride [(¹³CH₃CO)₂O]. In blue and violet colors are shown the contours of the isotopic distributions corresponding to the peptides previously mentioned with a normal acetyl group (¹²CH₃CO)—and ¹³C-labeled acetyl group (¹³CH₃CO)₂O], respectively. The contour of the isotopic distribution highlighted in red color show the adjustment carried out by the program after superimposing the isotopic distributions previously mentioned in the proportion (¹²CH₃CO-/¹³CH₃CO-) determined by the software as it is indicated for each particular peptide.

FIG. 6. The ESI-MS spectra in black color show the isotopic distributions of the peptides ²²¹DSSIVTHDNDIFR²³³ (rS K), ²⁸TGPNLHGLFGRK³⁹ (cytochrome-C) and ⁷⁹KGHHEAELKPLAQSHATK⁹⁶ (myoglobin) obtained after applying the method of selective isolation shown in the example 1 to an artificial mixture of three proteins (rSK, myoglobin and cytochrome-C) and using the reversible blocking of the amino groups with 2-(methyl sulfonyl) ethyl succinimidyl carbonate and the labeling with ¹⁸O at the carboxy C-terminal end during the proteolysis of the analyzed mixture of proteins. In blue, violet and yellow colors are shown the contours of the isotopic distributions corresponding to the previously mentioned peptides without incorporating ¹⁸O, after incorporating one atom of ¹⁸O (¹⁸O₁) and when incorporating two atoms of ¹⁸O (¹⁸O₂), respectively. The contour of the isotopic distribution highlighted in red color shows the adjustment carried out by the program after superimposing the isotopic distributions previously mentioned in the proportion (¹⁶O/[¹⁸O₁+¹⁸O₂]) determined by the software as it is indicated for each particular peptide.

FIG. 7. The graph shows the percentage of RH peptides from several proteomes, containing one, two, and three deuterated acetyl groups (C²H₃CO—) in their sequences.

FIG. 8. ESI-MSMS spectrum of the doubly-charged RH peptide at m/z 780.77 (²²¹DSSIVTHDNDIFR²³³) of the rSK that contains an acetyl group at their N-terminal end. The fragments ions were assigned automatically by the MASCOT programs according to the nomenclature proposed by Roepstorff P and Fohlman J (Roepstorff P and Fohlman J. Proposal for a common nomenclature for sequence ions in mass spectra of peptides. Biomed Mass Spectrom. 11, 984, 601).

FIG. 9 ESI-MSMS spectrum of a doubly-charged RH peptide at m/z 879.84 (³⁸FFEIDLTSRPAHGGK⁵²) of the rSK that contains two acetyl groups one located at their N-terminal end and the other one at the amino group of the side chain of lysine residue. The fragments ions were assigned automatically by the MASCOT program according to the nomenclature proposed by Roepstorff P and Fohlman J (Roepstorff P and Fohlman J. Proposal for a common nomenclature for sequence ions in mass spectra of peptides. Biomed Mass Spectrom. 11, 984,601).

FIG. 10. Relative quantification of three proteins (rSK, cytochrome C and myoglobin) present in three compared samples. A peptide of each protein was selected to show the results obtained in the relative quantification using the ISOTOPICA software. The mass spectra highlighted in black color show the overlapping of the experimental isotopic distributions of the RH peptides of each one of the proteins present in the analyzed mixture. The peptides of the conditions “A”, “B” and “C” were labeled at their amino terminal end with CH₃CO—, ¹³CH₃CO—and ¹³CH₃¹³CO—, respectively. The contours of the isotopic distribution highlighted in blue, violet and yellow correspond to the RH peptides that do not have ¹³C, one atom of ¹³C and two atoms of ¹³C, respectively. The contour of the isotopic distribution highlighted in red color show the agreement that exists between the experimental isotopic distribution and the one obtained when superimposing the isotopic distributions of the conditions “A”, “B” and “C” in the proportions calculated by the ISOTOPICA software. In each one of the spectra the results are also provided by the software for the three labeled species.

### EXAMPLES

### Example 1

In silico demonstration of the simplification of complex mixtures of peptides derived from proteomes of different organisms using the chromatographic system proposed that allows the selective isolation of multiply-charged peptides (2+, 3+, 4 +, etc) or RH peptides.

The great efficiency reached by the sequencing of the DNA molecule has made possible that complete sequences of genomes of several organisms are know, and it is possible to predict which are the proteins that are derived from the genomes and what peptides are generated depending the specific proteolytic treatment that is carried out to the studied proteome. To have an exact idea about the magnitude of the simplification when the RH peptides are selectively isolated a program named SELESTACT was devised. It was written in C for use in PC and calculates from the proteome of a given organism:
1—the total number of proteins reported in the Swissprot database,
2—the total number of peptides/proteins that can be generated by an specific proteolytic treatment,
3—the number of RH peptides/protein that are generated by this specific proteolytic treatment,
4—the percentage of proteins that possess RH peptides (referred to the total number of proteins reported in particular proteome) that can be successfully identified by the method proposed in the present invention (proteome coverage).

This program also calculates all these parameters for the proteolytic peptides of a given proteome that possess cystein residues and therefore they can be selectively isolated by the ICAT method, one of the pioneering and the most used method in the selective isolation of peptides and its application to proteome studies

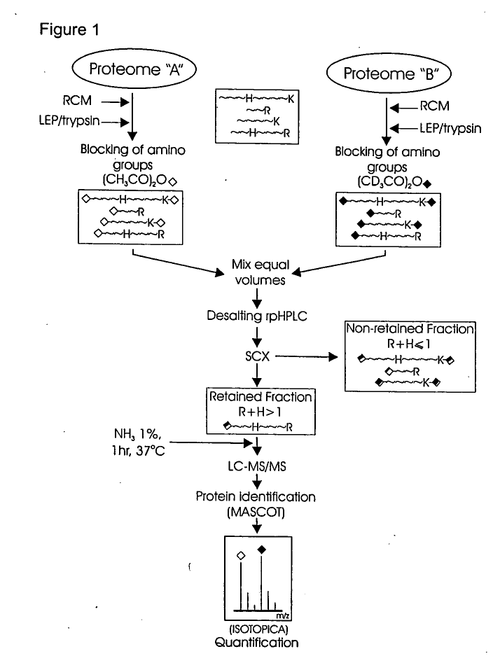

TABLE 1
In silico analysis of proteomes of different organisms by using of
the SELESTACT software to determine the contained RH peptides when
applying the method proposed in the FIG. 1 that uses a chromatographic
system that separates the neutral and singly-charged peptides (charge
0 and 1+) from the multiply-charged peptides (charge 2+, 3+,
4+, etc) or RH peptides. The results for the simplification when
applying the well-known method ICAT, are also shown.

| | | | Peptides | |
|---|---|---|---|---|
| | | | selectively | |
| | | Total tryptic | isolated/ | Coverage of |
| Analyzed | total # of | peptides/ | protein ^{c)} | the proteome ^{d)} |
| Proteome | proteins ^{a)} | protein ^{b)} | (RH/ICAT) | (RH/ICAT) |
| N. meningitidis | 1967 | 14 | 3/2 | 80.8/81.6 |
| V. cholerae | 3785 | 15 | 3/2 | 83.9/80.3 |
| M. tuberculosis | 3875 | 15 | 5/2 | 94.6/79.0 |
| E. coli | 4322 | 15 | 4/3 | 87.7/83.2 |
| S. cerevissiae | 4818 | 24 | 4/4 | 88.0/89.2 |
| A. thaliana | 26003 | 14 | 4/5 | 90.3/93.2 |
| M. musculus | 28959 | 14 | 5/6 | 91.9/94.7 |
| H. sapiens | 47531 | 22 | 6/6 | 92.5/94.8 |
| Average | — | 18 | 4/4 | 87.9/87.0 |

^{a)} it corresponds to the total number of proteins reported in the Swissprot sequences database.
^{b)} total number of tryptic peptides/proteins that are generated by the specific proteolysis with trypsin of the different analyzed proteomes. It is expressed as a integer number.
^{c)} total number of multiply-charged peptides/analyzed protein when using the chromatographic system described in heading of the this table and the one obtained by the ICAT method. Both data are expressed as integer numbers.
^{d)} The coverage of the analyzed proteome represents the percentage of the total number of proteins that possess RH peptides and can be isolated when using the chromatographic method described in the heading of the present table. The values correspond to the proposed method and the ICAT.

Table 1 summarizes the results obtained by in silico analysis after applying the method to several proteomes including bacteria pathogen bacteria, yeasts, plants, and mammals are shown. As it can appreciate, of an average of 18 tryptic peptides per protein, the mixture would be simplified considerably until reaching an optimum value for these purposes because 4 RH peptides/proteins are selectively isolated at the same extent as the ICAT method. The average for the proteome coverage, that represents the percentage of the proteome of an organism that can be studied with the proposed method (87.9%) it is also very similar to the one achieved with the ICAT (87%). However, when individual proteomes are analyzed a very marked difference it is clearly noticed for the case of M. tuberculosis proteome. In the method object of the present invention 94,6% of the complete proteome can be analyzed, on the other hand, when the ICAT method is used only it can analyze less than 80%. When studying particularly this microorganism in proteomics the method of selective isolation object of this invention it should be the method of choice. The usage of the SELESTACT software is of great utility to predict which are the expected results from the method object of the present invention for particular organism and therefore it allows the comparison with other methods of selective isolation of peptides for proteomic studies.

This demonstrates us that the principle of this invention, the separation of peptides with charges 0 and 1+from the multiply-charged peptides (charges 2+, 3+, 4+, etc) by using a chromatographic system it is of great utility and it can be successfully used for the proteomics study of organisms with different evolutionary grades since it allows an ideal simplification of the complex mixture of peptides and at the same time it guarantees high coverage of the proteome under study.

It only remains to demonstrate in the example 2, a chromatography system that is able to make in a simple and effective way of the proposed separation, the neutral and singly-charged peptides from the multiply-charged peptides.

### Example 2

Selective Isolation of the RH peptides of the recombinant streptokinase (rSK). Evaluation of the selectivity and the specificity of the method.

The method proposed in the FIG. 1 was applied to the rSK protein. This protein was selected as model because its tryptic digest generates a great number of tryptic peptides and RH peptidases well. Therefore it is very easy the evaluation of the specificity and the selectivity of the proposed method.

The steps to follow are shown below:
1. the protein was dissolved in a 500 mM Hepes buffer (pH 8.0) containing guanidium chloride (2 mol/L) and lysyl-endopeptidase was added to a enzyme:substrate ratio 1:200 and the digestion proceeded at 37° C. during 16 h. Next, the digestion buffer was diluted until obtaining a concentration of 1 mol/L of guanidium chloride, and the trypsin was added to a enzyme:substrate ratio of 1:100 and the digestion proceeded additionally during 4h to 37° C.
2. the mixture of proteolytic peptides were incubated at a temperature between 0-5° C., and acetic anhydride was added in a molar ratio 10:1 with respect to the primary amino groups (alpha and epsilon). The mixture was shortly stirred and it was incubated again in a ice bathroom. This procedure was repeated additionally twice at intervals of five minutes.
3. the excess of reagent was eliminated by reverse phase chromatography using a RP-C4 column (Vydac, 20 x 2.1 mm) previously equilibrated in a solution of H₂O/TFA 0.05% (v/v) and the peptides are collected in a single fraction when carrying out a fast gradient in 10 minutes by increasing the content of acetonitrile containing 0.05% (v/v) TFA from 1 to 80% of the mobile phase.
4. the peptides blocked at the primary amino groups are applied to strong cation exchange column (for example: Source 30S, 2×20 mm, Amersham-Pharmacia, Sweden) previously equilibrated with a solution of H₂O/TFA (0.05% v/v) that contains 15 % of acetonitrile using a flow of 200 microliters/min. The non-retained fraction that contains neutral (R+H=0) or singly-charged peptides (R+H=1) is discarded, on the other hand, the retained fraction that contains to the multiply-charged peptides (RH, R+H>1) is additionally fractioned by increasing the saline concentration of the equilibrium solution.
5. before being analyzed by means of liquid chromatography coupled to mass spectrometry, the RH peptides is treated at basic pH (1% of triethylamine during 1 hour at 37° C.) to eliminate from its structures the non-specific blockages (O-acylations) present at the tyrosine residues. In this step, the hydrolytic treatment used to eliminate those O-acylations as well as the blocking group (when a reversible blockage is used at the amino groups) should not eliminate the labeling introduced since it is indispensable to carry out the relative quantification of peptides.

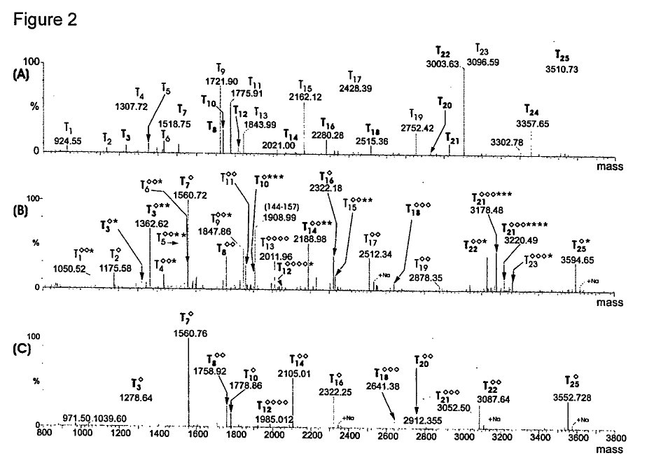

The ESI-MS spectrum (FIGS. 2A) shows a considerable number of signals corresponding to the peptides generated during the tryptic digestion of the rSK. The assignments of these signals to the sequence of the rSK (sequence 1 at the end of the document) are shown in the Table 2, as well as the experimental and theoretical mass values for each peptide. In this table the peptides were classified regarding to the number of positive charges that they can have at acidic pH once their primary aminos groups have been acetylated. Logically, the number of positive charges (see Z, Table 2) depend on the number of arginine (R) and/or histidine (H) residues contained within their respective sequences (R+H). As it can be can observed in Table 2, 12 out of the 25 signals correspond to peptides that once blocked the primary amino groups at acidic pH would be neutral (z=0, having neither arginine nor histidine in their respective sequences) or singly-charged (z=1, to have an arginine residue or a histidine residue in their sequences).

Those signals with assignments written in boldfaces correspond to 13 multiply-charged peptides or RH peptides (peptides T₃, T₇₋₈, T₁₀, T₁₂, T₁₄, T₁₆, T₁₈, T₂₀₋₂₂, T₂₄₋₂₅, FIG. 2A). These peptides fulfills the condition that in their sequences the sum of the number of arginine and/or histidine residues are superior than one (R+H>1).

The primary amino groups (amino terminal and epsilon amino groups of lysines) of tryptic peptides were blocked and it is observed all increased their molecular masses in dependence of the number of acetyl groups added to their structure (see number of added rhombuses, in FIG. 2B). However, some peptides incorporated additional acetyl groups at the tyrosine residues (FIG. 2B, peptides 3-6, 9-10,12,14, 16, 21-22 and 25). This mixture is simplified considerably after passing through the cation exchanger: only 12 peptides out of 25 that had been detected initially. Later on, this undesirable modification at the tyrosine residues (Zappacosta F, and Annan RS. N-terminal isotope tagging strategy for quantitative proteomics: results-driven analysis of protein abundance changes. Anal Chem. 2004, 76, 6618-6627) was eliminated with a basic treatment mentioned in the step 5 of the method (FIG. 2C). The non-retained fraction in the cation exchange chromatography was discarded and the retained fraction was eluted in a single step by increasing the concentration of NaCI in the mobile phase to 2 mol/L. Notices, that the expected simplification is achieved for the mixture of peptides derived from the rSK and 12 out of the 13 peptides classified as RH in the FIG. 2A, were isolated successfully in the FIG. 2C (peptides T₃, T₇₋₈, T₁₀, T₁₂, T₁₄, T₁₆, T₁₈, T₂₀₋₂₂, T₂₅) for 92 % selectivity. Notice also that in th ESI-MS spectrum any of those neutral peptide (z=0) or singly-charged (z=1) that appear in the Table 2 were not detected in the spectrum of the FIG. 2A, what is equal to 100% of specificity.

On the other hand, none of the multiply-charged or RH peptides shown in the spectrum of the FIG. 2C contain additional acetylations (asterisks) respect to the expected ones, therefore the basic treatment to eliminate these O-acylations at the tyrosine residues worked in a quantitative way.

TABLE 2
Summary of the assignment of the signals observed in
the FIG. 2A for the tryptic peptides of the rSK.

| | | | | m/z |
|---|---|---|---|---|
| Code ^{a)} | Sequence assignment ^{b)} | z ^{c)} | m/z exp ^{d)} | theor. ^{e)} |
| T1 | ³⁶⁵IITVYMGK³⁷² | 0 | 924.55 | 924.52 |
| T2 | [symbol] | 1 | 1133.62 | 1133.57 |
| [symbol] | [symbol] | [symbol] | 1236.64 | 1236.59 |
| T4 | ³⁰⁰YVDVNTNELLK³¹⁰ | 0 | 1307.72 | 1307.68 |
| T5 | [symbol] | 1 | 1351.71 | 1351.66 |
| T6 | ⁴⁰³YTGTPIPDNPNDK⁴¹⁵ | 0 | 1431.71 | 1431.67 |
| [symbol] | [symbol] | [symbol] | 1518.76 | 1518.71 |
| [symbol] | [symbol] | [symbol] | 1674.92 | 1674.85 |
| T9 | [symbol] | 1 | 1721.90 | 1721.85 |
| [symbol] | [symbol] | [symbol] | 1736.87 | 1736.81 |
| T11 | ²⁵⁹SGLNEEINNTDLISEK²⁷⁴ | 0 | 1775.91 | 1775.86 |
| [symbol] | [symbol] | [symbol] | 1816.99 | 1816.93 |
| T13 | [symbol] | 1 | 1843.99 | 1843.93 |
| T14 | [symbol] | [symbol] | 2021.04 | 2020.94 |
| T15 | ³³⁶LLYNNLDAFGIMDYTLTGK³⁵⁴ | 0 | 2162.12 | 2162.08 |
| [symbol] | [symbol] | [symbol] | 2280.28 | 2280.19 |
| T17 | ¹⁸⁸TLAIGDTITSQELLAQAQSILNK²¹⁰ | 0 | 2428.39 | 2428.32 |
| [symbol] | [symbol] | [symbol] | 2515.36 | 2515.29 |
| T19 | [symbol] | 1 | 2752.42 | 2752.34 |
| [symbol] | [symbol] | [symbol] | 2828.20 | 2828.28 |
| [symbol] | [symbol] | [symbol] | 2926.52 | 2926.44 |
| [symbol] | [symbol] | [symbol] | 3003.63 | 3003.55 |
| T23 | [symbol] | 1 | 3096.59 | 3096.51 |
| [symbol] | [symbol] | [symbol] | 3357.65 | 3357.56 |
| [symbol] | [symbol] | [symbol] | 3510.73 | 3510.66 |

^{a)} Code corresponding to the tryptic peptides of the rSK observed in the FIG. 2A. The codes highlighted in boldfaces and italic correspond to the RH peptides.
^{b)} The amino acids highlighted in boldfaces and italic are the arginine and histidine residues in the sequence of the tryptic peptides of rSK. The numbering of the amino acids N - and C- terminal correspond with the position of each one of them in the sequence of the rSK.
^{c)} Z is the number of positive charges holded by the arginine and histidine residues in the peptides dissolved at acidic pH.
^{d)} experimental mass of the tryptic peptides of the rSK.
^{e)} theoretical mass of the tryptic peptides of the rSK.

These results demonstrate that the chromatographic system designed is very specific and selective to isolate the multiply-charged or RH peptides and it can be applied with success in the proteomic studies.

### Example 3

Identification and relative quantification of the component proteins of two artificial mixtures (A and B) by means of the selective isolation of RH peptides by using three different isotopic labeling (²H, ¹³C and ¹⁸O).

Two artificial mixtures A and B composed of the proteins rSK, myoglobin and cytochrome-C were prepared in a molar ratio of A with respect to B: 1:1 for the rSK, 2:1 for the cytochrome C and 1:3 for the myoglobin. The sequences of these proteins are shown at the end of the document, sequences 1-3. The selective isolation of the RH peptides was carried out for the method described in the example 1. Three experiments were performed by using different isotopic labeling methods to demonstrate the compatibility of the proposed method with different kind of labeling (²H, ¹³C and ¹⁸O) and it is demonstrated the possibility of using the reversible blocking of the amino groups in case it is desirable to analyze in the mass spectrometer the peptides with their free amino groups.

### Experiment 1

The blocking of the primary amino groups of peptides obtained in the tryptic digestion of the mixture “A” was carried out using normal acetic anhydride ((C¹H₃CO)₂O) and the peptides of the mixture “B” were derivatized with deuterated acetic anhydride ((C²H₃CO)₂O) provided by ISOTEC (99% isotopic purity). In this experiment the molecular masses of the peptides obtained under both conditions differed in multiples of 3 units of masses depending the number of acetyl groups they added during the blocking reaction. In this experiment the overlapping of the isotopic distributions of the labeled and non-labeled peptides can be partial in case of a single acetyl group is added and the overlapping decrease as increase the quantity of blocking groups added to the peptides that contain lysine residues within their sequences.

### Experiment 2

The blocking of the primary amino groups of the tryptic peptides of the mixture “A” was carried out using normal acetic anhydride ((¹²CH₃CO)₂O) and those obtained from the mixture “B” were derivatized with ¹³C ((¹³CH₃CO)₂O, commercialized by ISOTEC 99% isotopic purity). In this experiment the overlapping of the isotopic distributions of the labeled and non-labeled peptides is elevated because their mass difference is just 1 Da for each blocking group added to a given peptide.

### Experiment 3

In this experiment some variants were introduced to the method described in the example 1 to demonstrate the combination of the ¹⁸O-labeling with the reversible blocking of the primary amino group of peptides. The mixture of proteins A was digested in a buffer described in the example 1 and the mixture B was digested with in the same buffer but prepared with heavy water (H₂¹⁸O) provided by ISOTEC (99% isotopic purity). By means of this procedure the peptides obtained in this last condition would be labeled with one or with two ¹⁸O atoms at their C-terminal end on the other hand, the other peptides keep their natural isotopic distribution. On the other hand, the amino groups of the tryptic peptides of both conditions were derivatized with a reversible reagent (2 (methyl sulfonyl) ethyl succinimidyl carbonate, marketed by Aldrich) and before mass spectrometric analyze they were eliminated in the same step of the removal of 0-acylations at the tyrosine residues using the same conditions described previously in the example 1.

In this case the analysis of the isotopic distributions is a more complex than in the previous cases because the peptides labeled with ¹⁸O can add 1 and 2 atoms of ¹⁸O at their C-terminal end, therefore for calculating the relative quantities of the peptides obtained under both conditions is important to take into account that the relationship is given by the quotient between the areas of the isotopic distributions of the peptides having ¹⁶O divided by the sum of the areas corresponding to the isotopic distributions of the peptides that incorporated one (¹⁸O₁) and two atoms of (¹⁸O₂), according to the expression:
(Area ¹⁶O)/(Area ¹⁸ O₁+Area ¹⁸O₂).

In all the above mentioned experiments the relative quantification of the peptides in the analyzed mixtures is carried out by using the ISOTOPICA software as it is explained in the detailed description of the method object of this invention (Fernández of Cossio et al. Isotopica, a Web Software for Isotopic Distribution Analysis of Biopolymers by Mass Spectrometry. Nucleic Acid Research 2004, 32, W674-W678 and Fernández of Cossio et al. Automated Interpretation of Mass Spectra of Complex Mixtures by Matching of Isotope Peak Distributions. Rapid Commun. Mass Spectrom. 2004; 18: 2465-2472).

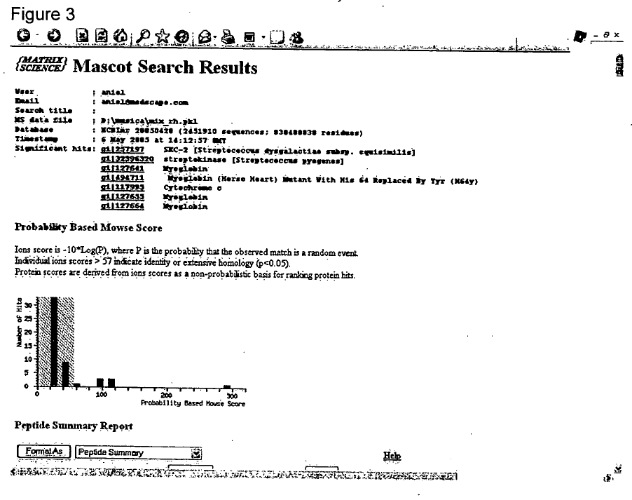

The RH peptides of the three proteins present in the prepared mixture were isolated and sequenced in a single LC-MS/MS experiment being able to identify automatically without ambiguities by the MASCOT program (it FIG. 3) the three proteins component of the artificially prepared mixture (rSK, cytochrome C and myoglobin). To proceed to the quantification, the expanded regions of the isotopic distribution of these peptides were selected and introduced in the ISOTOPICA software together with its global formulas and the type of labeling used. The results for the experiments 1, 2 and 3, where the labeling used were ²H, ¹³C and ¹⁸O, are summarized in the Table 3.

TABLE 3
Summary of the selective isolation of the RH peptides and their relative
quantification in two artificial mixtures composed by three proteins
using three different labeling: 1H/²H, ¹²C/¹³C and ¹⁶O/¹⁸O.

| | | Quantification | | | |
|---|---|---|---|---|---|
| | | | Experimental ^{c)} | | |
| | | | ¹H/²H | ¹²C/¹³C | ¹⁶O/¹⁸O |
| Proteins | Sequence of RH peptides ^{a)} | Theor. ^{b)} | Exp. 1 | Exp. 2 | Exp. 3 |
| rSK | [symbol] | 50:50 | 50.2:49.8 | 51.2:48.8 | 49.7:50.3 |
| | [symbol] | | 50.2:49.8 | 51.8:48.2 | 52.0:48.0 |
| | [symbol] | | 49.0:51.0 | 52.5:47.5 | 49.6:50.4 |
| | [symbol] | | 47.4:52.6 | 48.6:51.4 | 49.2:50.8 |
| | [symbol] | | 59.5:40.5 | 54.8:45.2 | 53.7:46.3 |
| | [symbol] | | 50.2:49.8 | 53.2:46.8 | 52.0:48.0 |
| | [symbol] | | 53.9:46.1 | 50.5:49.5 | 51.2:48.8 |
| | | | 51.5:48.5 | 51.8:48.2 | 51.1:48.9 |
| | | | (±4.04) | (±1.99) | (±1.64) |
| Cytochrome C | [symbol] | 66:33 | 65.7:34.3 | 64.8:35.2 | 66.8:33.2 |
| | [symbol] | | 66.4:33.6 | 65.6:34.4 | 67.1:32.9 |
| | | | 66.0:34.0 | 65.2:34.8 | 66.9:33.1 |
| | | | (±0.49) | (±0.49) | (±0.21) |
| Myoglobin | [symbol] | 25:75 | 25.4:74.6 | 24.7:75.3 | 24.3:75.7 |
| | [symbol] | | 22.2:77.8 | 26.7:73.3 | 23.9:76.1 |

| | | |
|---|---|---|
| 24.1:75.9 | 25.7:74.3 | 24.1:75.9 |
| (±0.49) | (±1.0) | (±0.28) |

^{a)} Sequence of the RH peptides of the automatically identified proteins by the MASCOT software. The asterisks indicate acetylation in the epsilon amino group of lysine.
^{b)} theoretical proportion of the proteins in the compared artificial mixtures A and B.
^{c)} experimental value obtained for the relative quantities of the proteins present in the two compared artificial mixtures. In boldface letters is highlighted at the end of each experiment for each protein the average value of their relative quantification and the value of the standard deviation is indicated between parenthesis.

For the three proteins the average of the experimental values for the relative quantifications are in very good agreement with the theoretical values and the obtained standard deviation was very good (below 5%).

These results demonstrate that the method can be used in the quantitative proteomics for determining with very good accuracy the relative quantities of the proteins in mixtures.

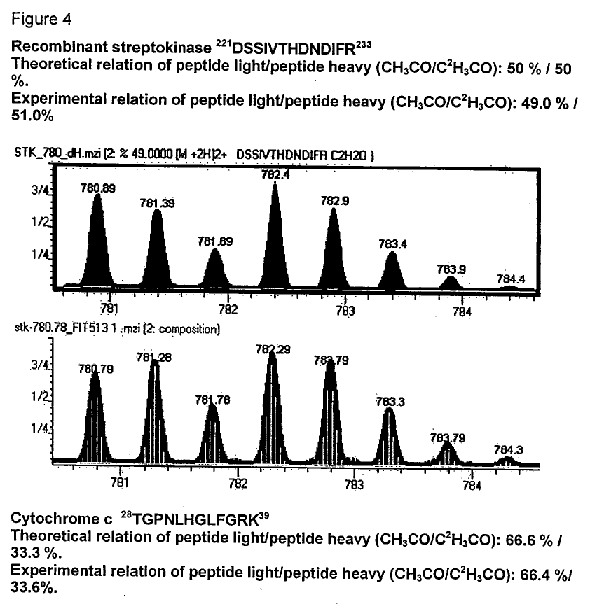

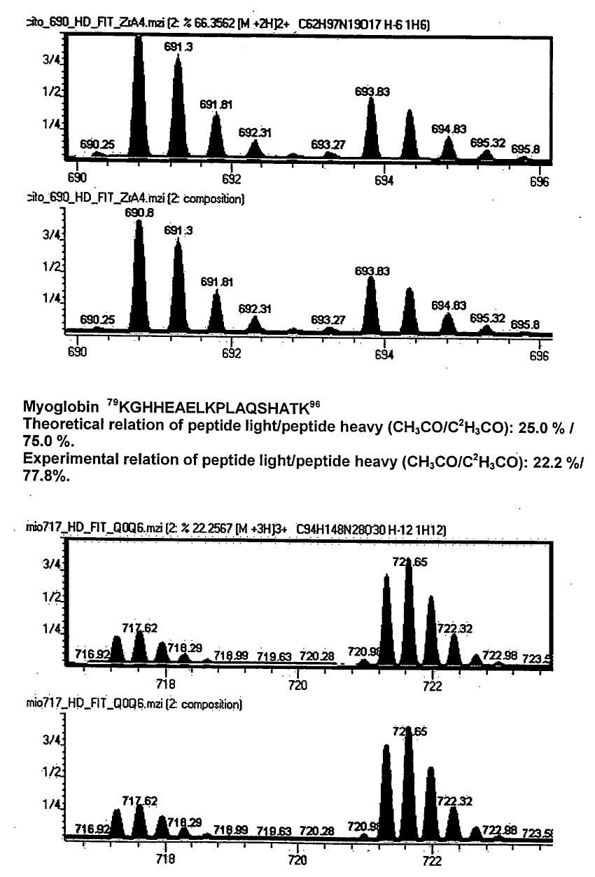

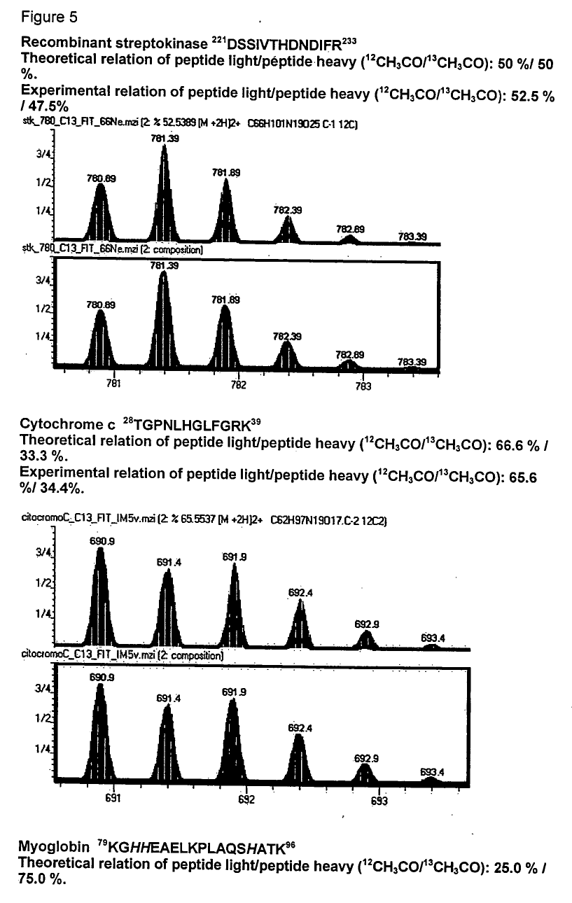

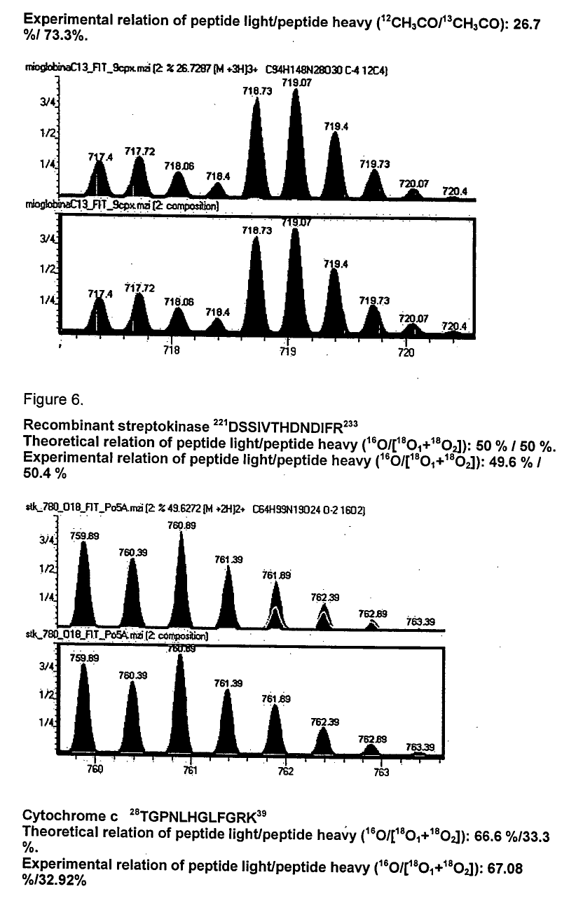

The adjustments of the areas corresponding to the experimental isotopic distributions obtained for one peptide of each one of the analyzed proteins in the three experiments mentioned in the present example are shown in the FIGS. 4, 5 and 6.

Notice that in all the cases a very good adjustment was obtained for the theoretical contour of the isotopic distributions (red line) and the spectrum obtained experimentally (spectrum shadowed in black) independently of the type of labeling used (either ¹³C, ²H or ¹⁸O).

The results obtained for the relative quantification of the same protein in the three experiments were very similar independently of the type of labeling used. This example demonstrates the versatility and robustness of the method due to its compatibility with different types of labeling to obtain a great accuracy in the relative quantification.

LC-MS/MS and Database Search.

The measurements were carried out in a hybrid mass spectrometer (quadrupole and time of flight, QTof-2) from the Micromass company (Manchester, United Kingdom). The mass spectrometer was connected online with a HPLC (AKTA Basic, Amersham Pharmacia Biotech, Sweden) through a column of 200×1 mm (Vydac, USES). The peptides were eluted with a lineal gradient from 5 to 45% of the buffer B (0.2% of formic acid in acetonitrile) in 120 minutes.

The mass spectrometer was operated with cone and the capillary voltages of 35 and 3000 volts, respectively. For the acquisition of the MSMS spectra the singly-, doubly-, triply-charged precursory ions were selected automatically, once these they surpassed an intensity of 7 counts/sec. The measurement mode was changed from MSMS to MS when the total ion current (TIC) diminished to 2 counts/sec or when the spectra MS/MS was acquired during 4 seconds. The acquisition and the data processing were carried out by the software MassLynx (version 3.5, Micromass), while the identification of the proteins based on the MSMS spectra was through the version of the MASCOT software freely available in Internet. Among the search parameters, the cytein modification as well as the possible oxidations and desamidations were included.

### Example 4

Application of the Proposed Method to the Quantitative Proteomics of Complex Mixtures Derived from the Proteome of an Organism (Vibrio cholerae) Grown in Aerobiosis and Anaerobiosis Conditions.

A colony of the strain of V. cholerae O1 biotype El Tor C7258 (Ogawa; Peru, 1991) grew during 16 h at 37° C. in a LB medium (Sambrok, J., Fritsch, E. F., Maniatis, T., Molecular cloning: to manual laboratory, Cold Spring Harbor Laboratory Press, New York 1989, A.1) was inoculated in 5 mL of syncase broth (pH 7.5) (Finkelstein, R.A., Attasampunna, A., Chulasamaya, M., Charunmethee, P., J. Immunol. 1966, 96, 440-449) suplemented with 0.4% w/v of glucose and 1% w/v of casein hydrolyzate and grown overnight to 200 rpm at 37° C.

The culture was diluted 1:100 in a fresh syncase medium suplemented and precultured until an OD˜0.2 before inoculating in the final aerobics and anaerobics culture used in the proteomics studies. The anaerobic atmosphere was generated using AnaeroGenTM sacks from the Oxoid company (Basingstoke, Hampshire, UK) until reducing the oxygen levels to values lower than 1% to generate a concentration of CO₂ between 9 and 13% during 30 min. Before proceeding to the definitive inoculation in the anaerobic culture, a 1 L erlenmeyer containing 200 mL of syncase was medium preconditioned during 30 minutes in anaerobiosis and later on it was inoculated with 2 mL of the strain C7258 precultivated and stirred to 220 rpm at 37° C. until reaching an optic density of approximately 0.5. The cells were collected by centrifugation to 10 000 g during 5 minutes, washed twice with the electroporation buffer (270 mM sucrose, 1.3 mM Na₂HPO₄, 1 mM MgCI₂, pH 7.4), resuspended again in the same buffer, and centrifuged during three minutes at 10 000 g. Aliquot of 10¹⁰ cells were stored at −70° C. Two aliquots (1×10⁷ cells) from the aerobic and anaerobic culture were dissolved separately in 1 mL of the lysis buffer (500 mM HEPES (pH 8.0), containing 10 mM EDTA, 2 mol/L guanidinium chloride). The complete disruption of the cells was carried out alternating ultrasound cycles (1 minute) and the incubation in a bathroom of ice. This procedure was repeated three times and later on the sample was pipeted continually several times until diminishing the viscosity of the solution. Finally streptomycin sulphate was added (Merck, Germany) until reaching a concentration of 1%, it was incubated during 15 minutes to 0° C. again, it was centrifuged at 500 g and the precipitate was discarded.

Later on, the complex mixture was analyzed following the steps described in the FIG. 1. The results of the identification and relative quantification are shown in the Table 3. Only two out of the 137 identified peptides do not classify as RH peptides (NPDGEVLR / protein No.62; and TRDNEVVAK */ protein No.72). These results confirm the high grade of specificity (superior to 98%). These results could be used to restrict the search in the protein sequence databases only to RH peptides and this contributes to avoid false-postives in the identifications.

Of the 91 identified proteins using the method of the present invention, 18 (it is equivalent to 19.8%) do not contain cystein in their amino acid sequences (proteins #2, 12, 13, 20, 22, 28, 29, 40, 41, 42, 44, 47, 48, 52, 60, 62, 68, and 88, see Table 4) and therefore they could not be identified using one of the most used methods in the quantitative proteomics, the ICAT (Quantitative analysis of complex protein mixtures using isotope-coded affinity tags. Gygi, S. P., Rist, B., Gerber, S. A., Turecek, F., Gelb, M. H., and Aebersold, R. Nat. Biotechnol. 17, 994-999, 1999; Simplification of complex peptide mixtures for proteomic analysis: reversible biotinylation of cysteinyl peptides. Spahr C S, Susin INC, Bures E J, Robinson J H, Davis M T, McGinley M D, Kroemer G, Patterson S D. Electrophoresis, 21(9), 1635-1650, 2000). Aomng the ten proteins that changed their expression levels identified by the present method (proteins #8, 9, 33, 43, 46, 55, 58, 70, 88 and 89, Table 4) one of them (10%) could not be identify with the ICAT because it does not have cystein residues in its sequence (protein 88, Table 4) and this valuable information would be lost if the ICAT method is used.

These results demonstrate the superiority of the present method to study complex mixtures of proteins and it allows to identify non identifiable proteins by an established method such as the ICAT.

TABLE 4
Summary of the selective isolation of the RH peptides present in hydrolysis of the
total proteins of Vibrio cholerae grown in aerobic and anaerobic conditions.

| | | | | Mass (peptide) | |
|---|---|---|---|---|---|
| # | Protein (MW/pI) | Peptides (sequence) ^{a)} | z | m/z exp_theor | % (d0_d3) ^{b)} |
| 1 | phospho-2-dehydro-3- | AHQELIPFLHNR | 3 | 506.26_506.23 | 48.84_51.15 |
| | deoxyheptonate aldolase, tyr- | | | | |
| | sensitive, VC0695 (39743/6.36) | RQPLVAEDVIHQIR | 3 | 572.60_572.64 | 51.37_48.63 |
| 2 | ribosomal protein L21, | VVAEVVQHGR | 2 | 568.30_568.25 | 57.2_42.8 |
| | gi\|15640462, VC0435, (11542/9.99) | | | | |
| 3 | inositol monophosphate family | AILQHIR | 2 | 446.76_446.73 | 61.20_38.79 |
| | protein, VC0745 (31638/7.66) | | | | |
| 4 | ribosomal protein L28, VC0218 | FLPNLQTHR | 2 | 584.27_584.31 | 58.34_41.65 |
| | (9022/11.61) | RPAVGNNR | 2 | 463.20_463.25 | 63.0_37.0 |
| 5 | transcriptional activator HlyU, | AMIELLHR | 2 | 512.26_512.77 | 55.00_44.99 |
| | VC0678 (12327/8.66) | | | | |
| 6 | elongation factor TU, VC0362 | EHILLGR | 2 | 440.21_440.25 | 55.32_44.37 |
| | (43141/5.04) | TK*PHVNVGTIGHVDHGK* | 3 | 627.28_627.33 | 54.02_45.98 |
| | | | 2 | 961.46_961.50 | 57.24_42.76 |
| 7 | valyl-tRNA synthetase, VC2503 | QLWWGHR | 2 | 514.22_513.24 | 44.76_55.23 |
| | (108144/5.1) | | | | |
| 8 | pyruvate dehydrogenase, E1 | EFEYLHAR | 2 | 554.22_553.76 | 75.3_24.7 |
| | component, VC2414 | ALHGYTPQR | 2 | 542.74_542.78 | 72.7_27.3 |
| | (98951/9.52) | HDVDALETQEWLAALESVVR | 3 | 774.99_775.06 | 75.45_24.55 |
| 9 | pyruvate dehydrogenase, E2 | ISGANLHR | 2 | 455.21_455.24 | 76.97_23.02 |
| | component, dihydrolipoamide | | | | |
| | acetyltransferase, VC2413 | | | | |
| | (66077/5.04) | | | | |
| 10 | peptidoglycan-associated | VTIEGHADER | 2 | 584.73_584.77 | 48.00_51.99 |
| | lipoprotein, VC1835 (65966/4.94) | | | | |
| 11 | phosphoribosylformylglycinamidine | MPIAVSHGEGR | 2 | 598.25_598.29 | Espec_Inco |
| | synthase, VC0869 (141374/5.12) | | | | |
| 12 | ribosomal protein L22, VC2591 | RSSHITVVVADR | 3 | 461.21_461.25 | 58.45_41.54 |
| | (12233/9.91) | | | | |
| 13 | ribosomal protein L1, VC0325 | GATVLPHGTGR | 2 | 554.24_554.29 | 58.0_42.0 |
| | (2644/9.61) | | | | |
| 14 | DNA-directed RNA polymerase | IHTEEDERPIGR | 3 | 498.53_498.57 | 59.17_44.82 |
| | alp, VC2571 (36515/4.85) | | | | |
| 15 | ribosomal protein L2gi\|15642588, | GVRPTVR | 2 | 413.70_413.74 | 59.65_40.34 |
| | VC2593 (29849/10.83) | GVVMNPVDHPHGGGEGR | 3 | 592.59_592.55 | 63.6_36.4 |
| | | ATIGEVGNAEHM*LR | 2 | 778.30_778.38 | 57.88_42.12 |
| | | ATIGEVGNAEHMLR | 2 | 770.34_770.38 | 52.37_47.63 |
| 16 | Amidophosphoribosyltransferase, | AISNVHR | 2 | 419.70_419.72 | 60.24_39.75 |
| | VC1004 (55904/5.75) | | | | |
| 17 | spermidine/putrescine ABC | EVFHIALR | 2 | 513.73_513.78 | 56.35_43.65 |
| | transporter, periplasmic | | | | |
| | spermidine/putrescine-binding | | | | |
| | protein, VC1425 (40797/4.95) | | | | |
| 18 | heat shock protein HtpG | GTDIILHLR | 2 | 540.30_540.31 | 39.5_60.5 |
| | gi\|15641000, VC0985 (72194/5.26) | | | | |
| 19 | rod shape-determining protein | MLQHFIR | 2 | 493.76_493.77 | 46.33_53.67 |
| | MreB, VC0415 (36920/5.04) | | | | |
| 20 | conserved hypothetical protein, | VTVTGSHSVR | 2 | 542.78_542.79 | 54.5_45.5 |
| | VC1055 (13395/5.72) | | | | |
| 21 | ribosomal protein L3, | RWNFR | 2 | 410.66_410.70 | 58.31_42.69 |
| | gi\|15642591, VC2596 (22387/9.81) | | | | |
| | | TQDMTHGNSLSHR | 3 | 509.16_509.23 | 63.53_36.47 |
| 22 | ribosomal protein S2, VC2260 | DMLTAGVHFGHQTR | 3 | 537.92_538.20 | 56.07_43.93 |
| | (26834/7.88) | | | | |
| 23 | ribosomal protein S1, VC1915 | RNNVVVSR | 2 | 493.27_493.22 | 59.8_40.2 |
| | (61057/4.86) | AFLPGSLVDVRPIR | 2 | 791.39_791.46 | 57.06_42.94 |
| 24 | DNA-directed RNA polymerase, | VIVSQLHR | 2 | 497.29_497.23 | 52.7_47.3 |
| | beta subunit, VC0328 (153253/5.22) | GESGLHPR | 2 | 447.69_447.73 | 56.3_43.7 |
| | | TLAHNLR | 2 | 433.71_433.75 | 54.26_45.74 |
| 25 | fructose-bisphosphate aldolase, | LNAISHR | 2 | 426.76_426.73 | 43.2_56.8 |
| | class II, VC0478 (38919/4.91 ) | YVHTVAEAYGVPVILHTDHAAK* | 3 | 825.70_825.76 | 38.95_61.05 |
| 26 | enolase gi\|15642443, VC2447 | DAGYTAVISHR | 2 | 616.29_616.30 | 34.75_65.4 |
| | (45820/5.03) | | | | |
| 27 | ribosomal protein S4, VC2572 | IDNAPGVHGAR | 2 | 574.73_574.78 | 48.0_51.9 |
| | (23387/9.99) | | | | |
| 28 | ribosomal protein L25, VC1640 | VEHM*DFIR | 2 | 552.75_552.71 | 57.65_42.35 |
| | (10525/6.82) | VEHMDFIR | 2 | 544.70_544.76 | 57.75_42.25 |
| | | VEHMDFIRI | 2 | 601.29_601.31 | 61.5_38.5 |
| 29 | ribosomal protein L32, VC2025 | HNVTAEGYYR | 2 | 626.22_626.28 | 63.24_36.76 |
| | (6334/11.29) | SHDALTAAALSVDATSGETHLR | 3 | 755.62_755.71 | 62.04_37.96 |
| 30 | ribosomal protein S21, VC0520 | VRENEPFDVALR | 2 | 743.88_743.81 | 60.48_39.52 |
| | (8524/11.33) | | | | |
| 31 | GDP-mannose 4,6-dehydratase, | LHYGDLTDSSNLTR | 2 | 817.38_817.39 | 49.56_50.44 |
| | VC0243 (42053/5.93) | | | | |
| 32 | lysyl-tRNA synthetase, heat | HYNPNHAQIQALTEEDIQNR | 3 | 812.70_812.64 | 38.56_61.43 |
| | inducible, VC0664 (58341/5.43) | LVMLLTNTHTIR | 2 | 727.34_727.41 | 40.49_59.51 |
| 33 | antioxidant, AhpC/Tsa family, | AYDVEHPEAGVAFR | 2 | 801.87_801.79 | 73.61_26.39 |
| | VC0731 (22889/5.37) | | | | |
| 34 | ribosomal protein S11, VC2573 | QVADGVAHIHASFNNTIVTITDR | 3 | 841.04_841.10 | 57.9_42.1 |
| | (13923/11.33) | | | | |
| 35 | elongation factor Tu, VC0321 | ARDFASIDNAPEER | 2 | 816.78_816.88 | 39.83_61.17 |
| | (43200/5.09) | TK*PHVNVGTIGHVDHGK* | 2 | 961.38_961.50 | 57.3_42.7 |
| 36 | acetyl-CoA carboxylase, carboxyl | HIFTEFDELAGDR | 2 | 796.32_796.37 | 48.63_51.37 |
| | transferase alpha subunit, | | | | |
| | VC2244 (35639/5.9) | | | | |
| 37 | DNA-directed RNA polymerase, | AEAIHYIGDLVQR | 2 | 763.82_763.78 | 52.96_47.04 |
| | alpha subunit, VC2571 (36435/4.83) | | | | |
| 38 | ATP synthase F1, alpha subunit, | EAFPGDVFYLHSR | 2 | 790.28_790.37 | 48.13_51.86 |
| | VC2766 (56789/5.54) | | | | |
| 39 | DNA-directed RNA polymerase, | EGLNVLQYFISTHGAR | 2 | 923.87_923.98 | 63.46_36.57 |
| | beta′ subunit, VC0329 (154967/ | | | | |
| | 6.48) | HIETIVR | 2 | 455.20_455.26 | 55.4_44.6 |
| 40 | Ribosomal protein S16, VC0561 | VNHWVSQGASLSDR | 2 | 798.80_798.30 | 60.24_39.76 |
| | (9119/10.47) | K*RPFYQIVVADSR | 2 | 831.84_831.95 | 49.48_50.52 |
| | | | 3 | 554.94_554.97 | 54.45_45.55 |
| 41 | Ribosomal protein S6, VC0366 | AHYVLM*NVEADQAVVDELETAFR | 3 | 893.35_893.43 | 51.41_48.69 |
| | (14682/5.96) | AHYVLMNVEADQAVVDELETAFR | 3 | 887.99_888.10 | 60.2_39.80 |
| | | HYEIVFMVHPDQSEQVAGMIER | 3 | 886.47_886.42 | 56.5_43.5 |
| 42 | phosphocarrier protein, VC0966 | EAVEHLVALM*DQL | 2 | 831.81_831.90 | 40.17_59.83 |
| | (9146/6.26) | EAVEHLVALMDQLH | 2 | 823.82_823.90 | 37.55_62.45 |
| 43 | Alcohol | IMWVMYEHPDTHFEELAMR | 3 | 827.28_826.36 | 25.7_74.3 |
| | dehydrogenase/acetaldehyde | NGIIFSPHPR | 2 | 590.75_590.85 | 23.27_76.73 |
| | dehydrogenase, VC2033, (96214 | QTAFSQYDRPQAR | 2 | 805.39_805.39 | 20.7_79.3 |
| | /6.08) | RAFLVTDR | 2 | 510.27_510.29 | 21.6_78.4 |
| 44 | ribosomal protein L4, VC2595 | EFNEALVHQVVVAYAAGAR | 2 | 1044.51_1044.00 | 47.4_52.6 |
| | (21897/9.76) | | | | |
| 45 | phosphoribosylamine-glycine | RVGDADTGPNTGGMGAYSPAPVVTQDVHDR | 3 | 1033.75_1033.47 | 58.7_41.3 |
| | ligase, VC0275 (45322/4.82) | | | | |
| 46 | thiamine biosynthesis protein | VTQLHYAR | 2 | 515.28_515.27 | 69.88_30.12 |
| | ThiC, VC0061 (72629/5.55) | | | | |
| 47 | ribosomal protein S7, VC0360 | VGGSTYQVPVEVRPVRR | 3 | 647.64_647.69 | 60.6_39.4 |
| | (17703/10.21) | AFAHYR | 2 | 403.66_403.70 | 63.11_36.89 |
| 48 | ribosomal protein S10, VC2597 | RTGAQVR | 2 | 415.24_415.20 | 59.4_40.6 |
| | (11742/9.69) | ERFTVLISPHVNK* | 2 | 812.38_812.45 | 58.6_41.4 |
| 49 | ATP synthase F1, gamma | VIGHVANASLEYR | 2 | 735.85_735.88 | 60.2_39.8 |
| | subunit, VC2765 (31862/8.34) | | | | |
| 50 | 2,3,4,5-tetrahydropyridine-2- | LQLISHR | 2 | 454.74_454.77 | 58.36_31.64 |
| | carboxylate N-succinyltransferase, VC2329 | | | | |
| | (35647/6.14) | | | | |
| 51 | asparaginyl-tRNA synthetase, | EFGIDPEHMDWYR | 2 | 868.82_868.87 | 57.3_42.7 |
| | VC1297 (52455/5.07) | | | | |
| 52 | DNA-directed RNA polymerase, | ERQEQQEQEAAELAAVSSIM*HNR | 3 | 904.70_905.76 | 52.8_47.2 |
| | omega subunit, VC2709 (10054/4.99) | | | | |
| 53 | chaperonin, 60 KDa subunit, | ASRPLLIVAEDVEGEALATLVVNNMR | 3 | 942.07_941.5 | 42.7_52.3 |
| | VC2664 (57153/4.78) | DRVEDALHATR | 3 | 442.20_442.22 | 43.20_56.80 |
| 54 | ribosomal protein S9, VC0571 | ALMEYDESLRPVLR | 2 | 867.38_867.44 | 56.74_43.26 |
| | (14686/10.67) | | 3 | 578.58_578.65 | 56.63_43.37 |
| 55 | formate acetyltransferase, | RAGAPFAPGANPMHGR | 3 | 550.22_550.28 | 21.94_78.05 |
| | VC1866 (87770/6.01) | EEIAEQHR | 2 | 527.22_527.24 | 21.6_78.4 |
| | | HMQFFGAR | 2 | 518.25_518.20 | 21.17_78.8 |
| | | RAIIPNGGIR | 2 | 555.28_555.32 | 17.8_82.2 |
| | | AGAPFAPGANPMHGR | 2 | 746.84_746.86 | 20.7_79.3 |
| 56 | 6-phosphofructokinase, isozyme | ATVLGHIQR | 2 | 518.75_518.79 | 38.44_61.56 |
| | I, VC2689 (35928/6.52) | MGNYAVHLLMEGHGGR | 3 | 596.24_595.60 | 48.3_51.7 |
| 57 | ribosomal protein S8, VC2582 | VSRPGLR | 2 | 413.71_413.75 | 58.63_41.37 |
| | (13981/10.01) | | | | |
| 58 | triosephosphate isomerase, | DFGASHIIIGHSER | 3 | 527.33_528.65 | 33.27_66.22 |
| | VC2670 (27971/5.03) | | | | |
| 59 | inorganic pyrophosphatase, | AQITHFFER | 2 | 595.75_595.79 | 65.68_34.31 |
| | VC2545 (19630/4.87) | | | | |
| 60 | ribosomal protein L19, VC0564 | GLHSAFTVR | 2 | 515.22_515.27 | 57.65_42.34 |
| | (13278/10.23) | TFQTHSPVVDSIEVK* | 2 | 864.89_864.94 | 53.6_46.4 |
| 61 | Elongation factor Tu-A (EF-Tu-A), | EHILLGR | 2 | 440.21_440.25 | 62.03_37.96 |
| | VC0321, (43185/5.09) | GYRPQFYFR | 2 | 638.23_638.32 | 56.57_43.43 |
| 62 | MSHA biogenesis protein MshN, | NPDGEVLR | 2 | 471.22_471.24 | 60.43_39.57 |
| | VC0404 (42853/6.24) | | | | |
| 63 | threonyl-tRNA synthetase, | ALQAHLTR | 2 | 476.21_476.26 | 55.52_44.27 |
| | VCA0287 (73443/5.92) | LIPVMIHR | 2 | 510.80_510.74 | 52.6_47.4 |
| 64 | thiamin biosynthesis protein | VTQLHYAR | 2 | 515.25_515.27 | 57.52_42.42 |
| | ThiC, VC0061 (72629/5.55) | | | | |
| 65 | Methylenetetrahydrofolate | DVDGFHPYNVGR | 2 | 709.29_709.32 | 53.3_46.7 |
| | dehydrogenase/ | | | | |
| | methenyltetrahydrofolate | | | | |
| | cyclohydrolase, VC1942 (33589/8.19) | | | | |
| 66 | NADH: ubiquinone | ATGPHAYLGR | 2 | 542.74_542.78 | 62.75_37.25 |
| | oxidoreductase, Na translocating, | | | | |
| | alpha subunit, VC2295 (50638/6.95) | | | | |
| | | YHLQVSVLR | 2 | 578.78_578.83 | 52.82_47.18 |
| 67 | DnaK protein, VC0855 (68761/4.82) | NQADQMIHATR | 2 | 663.79_663.81 | 53.07_46.93 |
| 68 | 50S ribosomal protein L18, | SGFQYHGR | 2 | 497.21_497.23 | 49.3_50.7 |
| | VC2580 (12685/10.97) | | | | |
| 69 | hypothetical protein, VCA0374 | K*VITSVHR | 2 | 512.25_512.30 | 61.4_38.6 |
| | (39390/4.93) | | | | |
| 70 | glyceraldehyde-3-phosphate | YDSTHGR | 2 | 439.19_439.16 | 32.7_67.3 |
| | dehydrogenase, VC2000 (35283/6.06) | | | | |
| 71 | ATP synthase alpha chain, | RQLTHGQK* | 2 | 526.26_526.29 | 45.4_54.6 |
| | VC2766 (55649/5.82) | | | | |
| 72 | ribosomal protein L17, VC2570 | TRDNEVVAK* | 2 | 537.28_537.26 | 54.8_45.2 |
| | (14437/11.07) | TDSVANRR | 2 | 480.72_480.75 | 60.1_39.9 |
| 73 | 30S ribosomal subunit protein | VHPNGIR | 2 | 417.71_417.73 | 56.7_43.3 |
| | S3, VC2590 (25596/10.63) | RAVQNAMR | 2 | 494.23_494.26 | 56.2_43.8 |
| 74 | superoxide dismutase, Fe, | HHNTYVVK* | 2 | 541.25_541.27 | 60.05_39.95 |
| | VC2045 (21483/5.39) | | | | |
| 75 | ribonuclease E, VC2030 (117525/7.47) | LVRPDFVNR | 2 | 579.31_579.32 | 54.82_45.18 |
| 76 | ATP synthase F1, beta subunit, | AIVMGSSDGLRR | 2 | 652.30_652.34 | 50.5_49.5 |
| | VC2764 (50520/4.85) | | | | |
| 77 | GMP synthase, VC0768 (57806/5.11) | HPFPGPGLGVR | 2 | 588.28_588.32 | 51.34_48.66 |
| 78 | NAD(P) transhydrogenase, alpha | AVVEAAHEFGR | 2 | 614.28_614.31 | 46.07_56.93 |
| | subunit, VCA0563 (54007/6.52) | | | | |
| 79 | elongation factor Ts, VC2259 | HVAMHVAASRPEFLTPDDVPAEVVAK* | 3 | 957.41_957.49 | 52.9_47.1 |
| | (30837/5.22) | HVAM*HVAASRPEFLTPDDVPAEVVAK* | 3 | 962.70_962.82 | 50.26_49.74 |
| 80 | asparagine synthetase B, | AILAHER | 2 | 426.22_426.24 | 54.02_45.98 |
| | glutamine-hydrolyzing, VC0991 | | | | |
| | (62362/5.70) | | | | |
| 81 | ribosomal protein L35, VCA0289 | RQLRPNAILPK* | 2 | 695.38_695.42 | 58.8_41.2 |
| | (7331/11.63) | RHILTK* | 2 | 426.20_426.26 | 66.96_33.04 |
| 82 | 2′,3′-cyclic-nucleotide 2′- | ALVEADATMVQALQHDHQGTR | 3 | 778.32_778.38 | 44.25_55.75 |
| | phosphodiesterase, VC2562 | | | | |
| | (75032/5.47) | | | | |
| 83 | glutamate-ammonia ligase, | YVVHNVAHAFGK* | 2 | 713.33_713.37 | 50.5_49.5 |
| | VC2746 (51751/4.99) | EQHISIPAHQIDADFFE [symbol] GK | 3 | 780.31_780.37 | 50.8_49.1 |
| | | GHRPGVK* | 2 | 471.68_417.73 | 52.3_47.7 |
| 84 | ribosomal protein L14, VC2586 | RPDGSVIR | 2 | 471.22_471.26 | 57.21_42.89 |
| | (13581/10.99) | | | | |
| 85 | carbamoyl-phosphate synthase, | ALREEGYR | 2 | 518.20_518.26 | 65.59_34.41 |
| | large subunit, VC2389 (117912/5.13) | AMEIVYDEQDLRR | 2 | 840.33_840.41 | 60.04_39.96 |
| | | AM*EIVYDEQDLRR | 2 | 848.32_848.40 | 63.55_36.45 |
| 86 | elongation factor G, VC2342 | VDVFTPEDHVGDVIGDLNRR | 3 | 765.64_765.72 | 56.06_43.94 |
| | (76482/5.06) | | | | |
| 87 | initiation factor IF-2, VC0643 | APVVTIM*GHVDHGK* | 2 | 780.82_780.90 | 57.26_42.74 |
| | (98705/5.73) | | | | |
| 88 | accessory colonization factor | FNIHPVFMR | 2 | 603.29_603.30 | 11.19_88.80 |
| | AcfC, VC0841 (28575/8.60) | | | | |
| 89 | succinate dehydrogenase, | LK*LDHLGR | 2 | 518.24_518.30 | 69.95_30.05 |
| | flavoprotein subunit, VC2089 | | | | |
| | (64510/6.45) | | | | |
| 90 | fructose-1,6-bisphosphatase, | YIGSLVADFHR | 2 | 660.31_660.34 | 49.8_50.2 |
| | VC2544 (37318/5.88) | | | | |
| 91 | phenylalanyl-tRNA synthetase, | GVDYALQHAAMER | 2 | 751.81_751.86 | 50.6_49.4 |
| | beta Chain, VC1220 (86079/5.09) | | | | |

^{a)} K* represents the acetylated lysine at the epsilon amino group. All the identified peptides possesses acetyl groups in the amino terminal end. N represents a completely desamidated asparagines residue. In boldfaces the basic amino acids arginine and histidine are highlighted.
^{b)} The peptides obtained in the aerobic and anaerobiosis conditions were derivatized with normal ((CH3CO)2O) and deuterado ((CD3CO)2O) acetic anhydrides, respectively. The proportion between both species is expressed by percentages.
^{c)} The sequences (8, 9, 33, 46, 89) and (43, 55, 58, 70, 88) correspond to the proteins overexpressed in anaerobiosis and aerobiosis conditions, respectively.

### Example 5

In Silico Determination of the Content of Blocking Groups Grupos in the RH Peptides of Different Genomes.

The presence of several blocking groups in the structure of the RH peptides when the irreversible blocking of the amino groups is used and in particular when deuterated acetic anhydride is used could be a concern mainly for three reasons:
1—As increase the number of blocking groups in the structure of the RH peptides, the losses during the whole process could be higher because they are transformed into more hydrophobic species.
2—As increase the number of blocking groups, the number of deuterium atoms in the sequence of the RH peptides will be higher and therefore the separation of the light and heavy species in reverse phase chromatography increases as well as the errors in their relative quantification under the compared conditions (Zhang R, Sioma C S, Wang S, Regnier F E. Fractionation of isotopically labeled peptides in quantitative proteomics. Anal Chem. 2001, 73, 5142-5149; Zhang R, Sioma C S, Thompson R A, Xiong L, Regnier F E. Controlling deuterium isotope effects in comparative proteomics. Anal Chem. 2002, 74, 3662-3669).
3—The presence of one or several blocking groups can affect the fragmentation of the RH peptides in the collisions induced dissociation experiments in the mass spectrometer.

All the RH peptides should possess a blocking group at least in its N-terminal end and the presence of additional blocking groups is due to the presence of lysine residues (for each lysine residual a blocking group is added). The presence of lysine in the RH peptides can originate for three reasons:
a) an incomplete proteolysis
b) presence of sites resistant to the proteolytic cleavage of trypsin due to the presence of a proline contiguous to the cleavage site (K-P).
c) RH peptides ending with lysine at their C-terminal end and possess inside their sequence multiple histidine and/or arginine residues.

The first of these factors (a) can be minimized by means of the optimization of the proteolytic digestion using a higher enzyme to substrate ratio or prolonged the digestion times.

The last factors (b and c) are an intrinsic property of each proteome and it depends on each one of the analyzed proteins, on the relative distribution of the proline residues contiguous to lysine residues (K-P) as well as of the presence of several basic residues in the tryptic peptides.

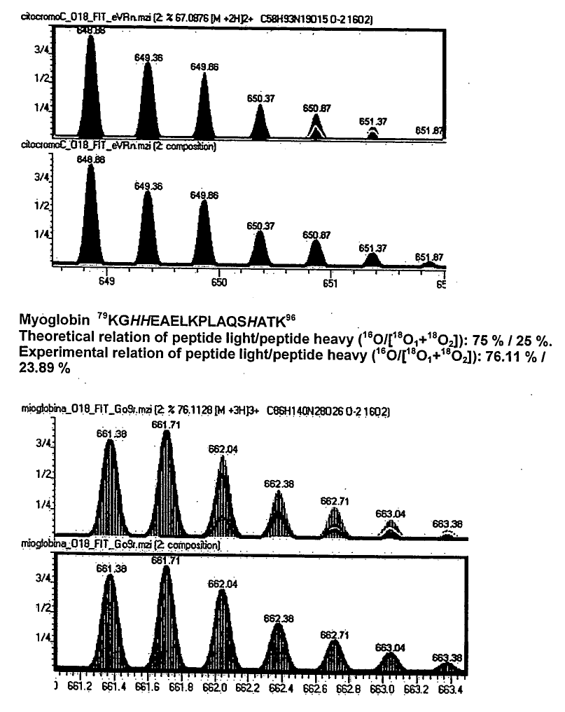

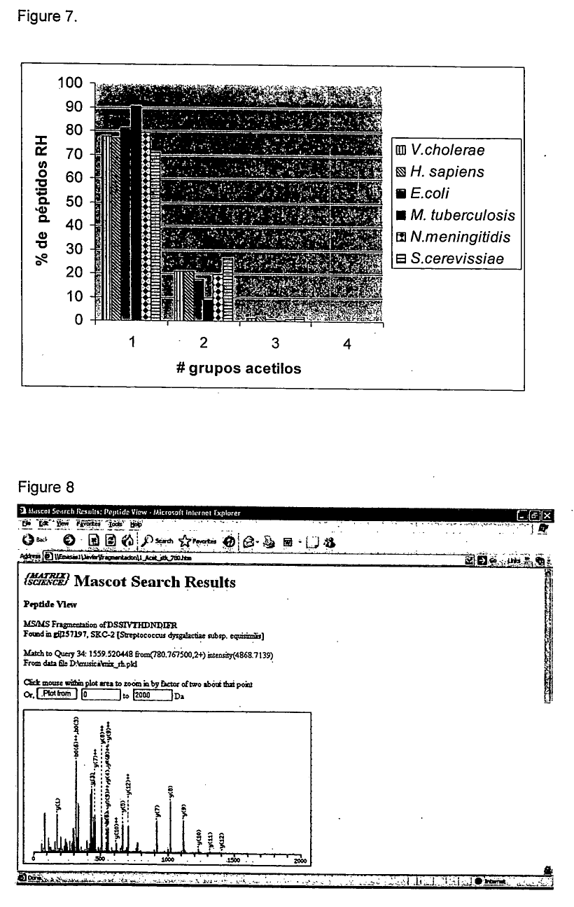

To know which is the abundance of K-P residues and the quantity of blocking groups, the sequences of all the RH peptides of several genomes were analyzed (FIGS. 7). The results demonstrate that approximately 80% of the RH peptides adds only one blocking group. Around 20% add two blocking groups and the addition of three or more blocking groups is irrelevant. Of the 137 RH peptides isolated experimentally in the proteome of V. cholerae grown in aerobic and anaerobic conditions (Table 4, example 4) it can be appreciate that 119 added only one acetyl group, 16 added two acetyl groups and only 2 RH peptides more than three, that is equivalent to a 86.8, 11.6 and 1.5%, respectively.

These results are similar to those obtained in the predictions carried out by an in silico analysis for the genome of this organism and similar to the other genomes shown in the FIG. 7.

Zhang and coworkers (Zhang R, Sioma C S, Wang S, Regnier F E. Fractionation of isotopically labeled peptides in quantitative proteomics. Anal Chem. 2001, 73, 5142-5149; Zhang R, Sioma C S, Thompson R A, Xiong L, Regnier F E. Controlling deuterium isotope effects in comparative proteomics. Anal Chem. 2002, 74, 3662-3669) they demonstrated that the presence of a single deuterated acetyl group does not affect the relative quantification of the labeled and non-labeled species, when deuterium labeling is used in the quantification it would not be a problem for our method since most of the peptides added one acetyl group (e deuterium atoms) but in the case of ICAT method all peptides adds 8 deuterium atoms and the errors in the quantification could be appreciable (Zhang R, Sioma C S, Wang S, Regnier F E. Fractionation of isotopically labeled peptides in quantitative proteomics. Anal Chem. 2001, 73, 5142-5149). The addition of two acetyl groups (6 deuterium atoms) present in the remaining 20% of the RH peptides of several genomes (FIG. 7) it would be even inferior to that added by all the ICAT-labeled peptides (8 deuterium atoms) and the errors using the method object of this invention will be inferior.

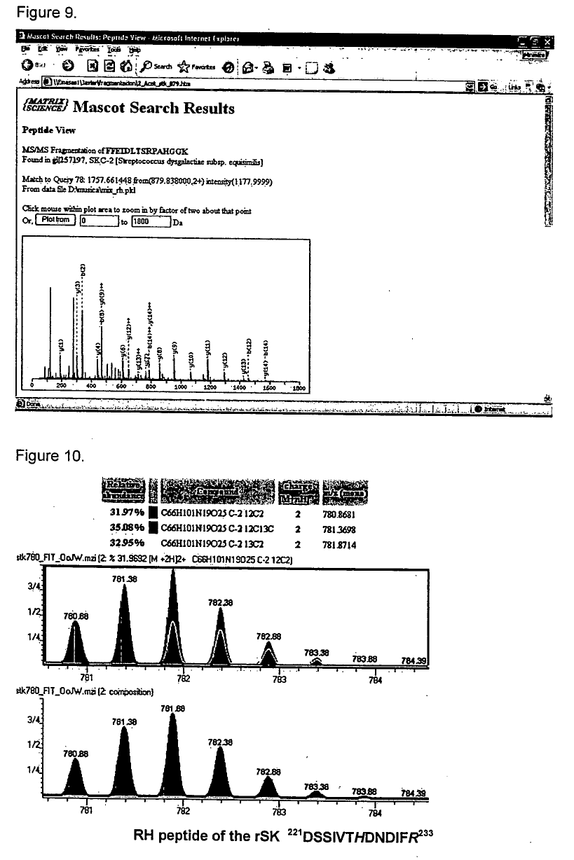

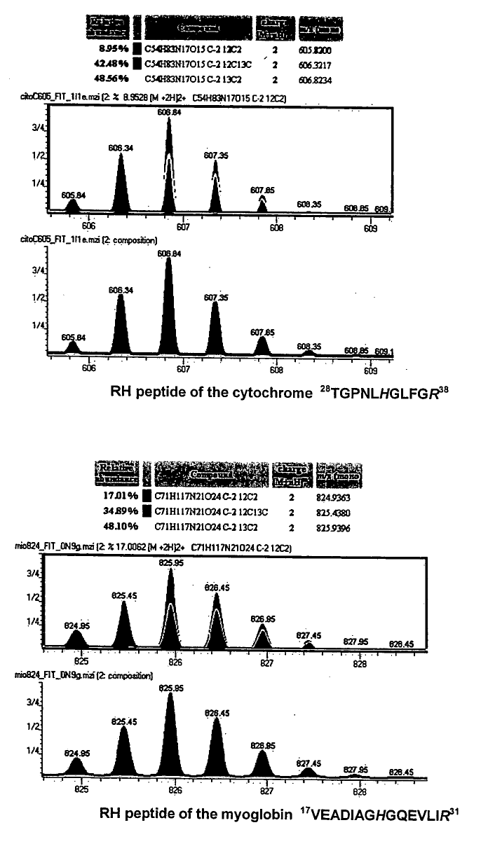

Lastly, the MASCOT software was able to identify in an automatic mode all the RH peptides of the rSK protein that possessed one and two blocking groups (FIGS. 8 and 9). This result demonstrates that the presence of multiple blocking groups in the sequence of the RH peptides does not affect the efficiency of its fragmentation in the collision induced dissociation experiments because the RH peptides are multiply-charged species (R+H>1) with multiple sites of protonation and consequently the automatic and efficient identification of the RH peptides is guaranteed in the protein sequence databases by using one of the most popular search engines, the MASCOT program.

### Example 6

Simultaneous Relative Quantification of Proteins Present in Several Simples in a Single Experiment.

The RH peptides isolated in the proposed method can be blocked at their N-terminal end with light and heavy isotopes to carry out the relative quantification. Taking into account, the results shown in the example 3 of the present invention, where it is demonstrated that the ISOTOPICA software can be used independently of the type of introduced labeling and the degree of overlapping of the isotopic distributions, in this example it is demonstrated that the relative quantification of proteins can be carried out in a simultaneous way under three or more biological conditions in a single experiment whenever a different isotopic labeling is used for the RH peptides in each one of the compared conditions.

Three artificial mixtures (A, B and C) composed by the proteins rSK, cytochrome-C and myoglogin were prepared. The proteins rSK, cytochrome C and myoglobine in the mixtures A, B and C kept the proportion of 1:1:1, 1:2:3 and 1:4:5, respectively. Later on, it proceeded to the isolation of the RH peptides of the proteins present in each one of the samples. The tryptic peptides of the proteins present in the mixtures A, B and C were derivatized with normal acetic anhydride [(CH₃CO)₂O], acetic anhydride labeled with an atom of carbon-13 [(¹³CH₃CO)₂O and acetic anhydride labeled with two atoms of carbon-13 [(¹³CH₃¹³CO)₂O], respectively. Before the desalting step, the three mixtures of blocked peptides were mixed and the procedure continued in the same way until the identification process. The blocked tryptic peptides of the proteins present in the mixture “C” are 1 heavier than the the blocked peptides of the mixture “B” and these in turn are 1 Da heavier than the peptides obtained in the mixture “A”. When A, B and C are mixed the isotopic distributions of the peptides from the same proteins are completely overlaped and the content of ¹³C is an reflect the relative quantities of the peptides present in each one of the compared conditions.

In the Table 5 are summarized the results obtained when applying the method for the selective isolation of RH peptides present in the artificial mixture of the three proteins. As they can be appreciated a very good agreement exists among the experimental isotopic distributions and those provided by the software when mixing in the appropriate proportions the theoretical distributions of the peptides. Particularly these values keep very good agreement with the expected values for each one of the proteins taking into consideration the proportions in which they were prepared in the three artificial mixtures.

TABLE 5
Simultaneous relative quantification of three proteins present in
three artificial mixtures when carrying out a single experiment.

| | | Theoretical | |
|---|---|---|---|
| | | ratio | Experimental |
| Proteins | Peptide Sequence ^{a)} | (A:B:C) | ratio |
| rSK | [symbol] | 1:1:1 | 1:0.94:0.98 |
| | [symbol] | | 1:1.14:1.17 |
| | [symbol] | | 1:1.06:1.03 |
| | [symbol] | | 1:0.99:1.02 |
| | [symbol] | | 1:1.13:1.09 |
| | [symbol] | | 1:0.90:0.94 |
| | [symbol] | | 1:0.83:0.95 |
| | | | 1:0.99:1.02 |
| Cytochrome-C | [symbol] | 1:4:5 | 1:4.30:5.2 |
| | [symbol] | | 1:4.40:5.5 |
| | | | 1:4.70:5.6 |
| Myoglobin | [symbol] | 1:2:3 | 1:2.02:2.86 |
| | [symbol] | | 1:2.18:2.99 |
| | | | 1:2.08:2.88 |

^{a)} In boldface the amino acids arginine and histidine are highlighted.

In the FIG. 8, the good adjustment carried out by the ISOTOPICA program for the peptides of each one of the proteins can be appreciated.

In principle the comparison can be extended to a greater number samples for which it is only necessary to introduce an isotopic labeling in each one of the conditions in a such way that their molecular masses are different in at least in 1 Da. For example, to be able to compare four conditions it would be necessary to use the same three anhydrides shown in this example and the RH peptides derived from the fourth condition should be modified with deuterated acetic anhydride [(C²H₃CO)O]. It is also possible to be compared 5 and 6 conditions if doubly labeled acetic anhydrides with deuterium and ¹³C ([(¹³C²H₃CO)O] and [(¹³C²H₃³CO)O],) are used, respectively. This application 20 would have great utility in proteomic experiments where it is necessary the comparison of multiple conditions either as internal controls of the system or to study synergistic effect of diverse drugs in cell lines, tumors, mircrorganisms, etc

If it is desirable to compare the expression of proteins under three different conditions using two-dimensional electrophoresis it would be necessary to obtain analytic three gels for each one of the conditions to evaluate the reproducibility of the experiment because of the exquisite manipulation that requires this analytic technique and later on, it would be necessary to obtain two preparative gels to proceed to the identification by mass spectrometry. It is evident, the great consumption of reagents, samples and time that it would require a study of such a magnitude. If LC-MSMS techniques are used, it would be necessary to carry out two experiments at least, when comparing one of the samples with the other two remaining ones. However, the possibility to introduce different labeling in the RH peptides isolated in each one of the conditions allows to obtain information of the relative quantification of each one of the proteins present in the mixtures in an single experiment. This minimizes the inter-experiments errors in the relative quantification and brings a considerable simplification of the work to be carry out. Particularly this advantage is more appreciable in the same measure as increase the number of samples to be compared.

1. Method for the identification and relative quantification of one or several proteins in complex mixtures, characterized because at acidic pH are selectively isolated the multply-charged peptides in those that the sum of the number of hisitidine residues (H) and/or arginine residues (R) in their sequences are higher than one (R+H>1), denominated here as RH peptides, and the determination of the relative concentration of the protein or the proteins is carried out from the ratio between the areas of theoretical sepectra estimated for each RH peptide labeled with different isotopes in the compared samples, which consists of the following steps:
a) Desnaturalizatión and and alkylation of the cystein residues of the proteins present in the analyzed complex mixture and proteolytic hidrolysis with trypsin or any other protease or chemical treatment that hidrolyze in a specific or predictable way the peptide bonds.
b) reversible or irreversible chemical modification of the alpha and epsilon amino groups (α and ε-NH₂) of the peptides obtained in the step (a)
c) selective isolation of the RH peptides by cation exchange chromatography of the mixture of modified peptides obtained in the step (b) previous to the analysis by mass spectrometry.
d) Elimination of O-acylations at the tyrosine residues and deblocking groups of the amino groups by an acidic or basic treatment previous to the analysis by mass spectrometry. In particular, the blocking of the amino groups is eliminated when a transitory or a reversible modification is introduced in the step (b).
e) Identification of the proteins by the mass spectromety analysis coupled to the liquid chromatography of the RH peptides selectively isolated in the step (c).
f) differential isotopic labeling of the samples of proteins previous to the step (a) or labeling of peptides during the steps (a) or (b), or during the hydrolysis step described in (d), mixing immediately at least a portion of the compared samples that contain identical quantities of proteins.
g) relative quantification of one or several proteins in the mixtures of the step (e) from the ratio between the areas of estimated theoretical mass spectra of the RH peptides identified in the step (e), as well as for the peptide fragments of these peptides generated in the same step (e) that contain the introduced isotopic labeling.

2. The method described in the claim 1, step (b), characterized because the modifying agents of amino groups could be: acetic anhydride, N-hydroxysuccinimide, N-acetoxysuccinimide, citraconic anhydride, maleic anhydride, succinic anhydride, ftalic anhydride, tetrahydroftalic anhydride, 9-fluorenylmethyl chloroformate (Fmoc-Cl), 2-methyl sulfonyl ethyl succinimidyl carbonate), urea and reagents that provides protecting amino groups such as:
(a) aromatic urethane-type protecting groups which include benzyloxycarbonyl, 2-chlorobenzyloxycarbonyl, 9-fluorenylmethyloxycarbonyl, isonicotinyloxycarbonyl and 4-methoxybenzyloxycarbonyl; (b) aliphatic urethane-type protecting groups which include t-butoxycarbonyl, t-amyloxycarbonyl, isopropyloxycarbonyl, 2-(4-biphenyl)-2-propyloxycarbonyl, allyloxycarbonyl and methylsulfonylethoxycarbonyl; (c) cycloalkyl urethane-type protecting groups which include adamantyloxycarbonyl, cyclopentyloxycarbonyl, cyclohexyloxycarbonyl and isobornyloxycarbonyl; (d) acyl protecting groups or sulfonyl protecting groups. Preferred protecting groups include benzyloxycarbonyl, t-butoxycarbonyl, acetyl, 2-propylpentanoyl, 4-methylpentanoyl, t-butylacetyl, 3-cyclohexylpropionyl, n-butanesulfonyl, benzylsulfonyl, 4-methylbenzenesulfonyl, 2-naphthalenesulfonyl, 3-naphthalenesulfonyl and 1-camphorsulfonyl; (e) photosensitive protective groups which include carbamates derivatives from m-nitrophenyl, 3,5-dimetoxybenzyl, 1-methyl-1 (3,5-dimetoxyphenyl)etyl, α-methylnitropiperonyl, o-nitrobenzyl, 3,4-dimetoxy-6-nitrobenzyl, phenyl(o-nitrophenyl)methyl, 2-(2-nitrophenyl)etyl, 6-nitroveratryl, 4-metoxyfenacyl and 3′,5′-dimetoxybenzoine and other reagents used in the pepetide synthesis for the protection reversible or irreversible of primary amino groups.

3. The method in agreement with the claim 1, step (c) characterized by the determination of the relative concentration of one or several proteins in the samples from their blocked or non-blocked RH peptides by calculating the ratio of the areas corrresponding to the theoretical spectra of the isotopically labeled and non-labeled species that are adjusted in the better way to the mass spectrum obtained experimentally or from the theoretical spectra of fragments of these peptides generated in the step (f) and that containing the introduced isotopic labeling.

4. The method in agreement with the claim 1, step (b) characterized by the usage of multiple modifier agents in a same experiment, so many as conditions to be analyzed.


SEQUENCE LISTING (94 sequences)

SEQ ID NO: 1 - protein, 415 aa, Streptococcus equisimilis
  PEPTIDE (1)..(415)
MIAGPEWLLDRPSVNNSQLVVSVAGTVEGTNQDISLKFFEIDLTSRPAHGGKTEQGLSPKSKPFATDSGAMPHKLEKADLLKAIQEQLIANVHSNDDYFEVIDFASDATITDRNGKVYFADKDGSVTLPTQPVQEFLLSGHVRVRPYKEKPIQNQAKSVDVEYTVQFTPLNPDDDFRPGLKDTKLLKTLAIGDTITSQELLAQAQSILNKTHPGYTIYERDSSIVTHDNDIFRTILPMDQEFTYHVKNREQAYEINKKSGLNEEINNTDLISEKYYVLKKGEKPYDPFDRSHLKLFTIKYVDVNTNELLKSEQLLTASERNLDFRDLYDPRDKAKLLYNNLDAFGIMDYTLTGKVEDNHDDTNRIITVYMGKRPEGENASYHLAYDKDRYTEEEREVYSYLRYTGTPIPDNPNDK

SEQ ID NO: 2 - protein, 153 aa, Equus caballus
  PEPTIDE (1)..(153)
VLSEGEWQLVLHVWAKVEADVAGHGQDILIRLFKSHPETLEKFDRFKHLKTEAEMKASEDLKKHGVTVLTALGAILKKKGHHEAELKPLAQSHATKHKIPIKYLEFISEAIIHVLHSRHPGDFGADAQGAMNKALELFRKDIAAKYKELGYQG

SEQ ID NO: 3 - protein, 104 aa, Equus caballus [sequence omitted]

SEQ ID NO: 4 - protein, 357 aa, Vibrio cholerae
  PEPTIDE (1)..(357): >tr|Q9KU34|Q9KU34_VIBCH Phospho-2-dehydro-3-deoxyheptonate aldolase, tyr-sensitive - Vibrio cholerae (357 aa).
MKKSELSDVNISNEQILITPDALKAKIPLSDKARKFIRESRQTVADIIHKRDPRLLIVCGPCSIHDVDAAKEYAKKLKALSAQLSDQLYIVMRVYFEKPRTTVGWKGLINDPHLDGSFDIEHGLHVGRQLLVDLAEMEIPLATEALDPISPQYLADTFSWAAIGARTTESQTHREMASGLSMPIGFKNGTDGNLATAINAMQAASSSHRFMGINREGQVALLTTQGNPNGHVILRGGKQTNYDSVSVAECEEEMQKARLEPSLMVDCSHANSRKDYRRQPLVAEDVIHQIREGNRSIIGLMIESHLNEGNQSSDLPREKMQYGVSITDGCINWSTTEALLRRAHQELIPFLHNRLQG

SEQ ID NO: 5 - protein, 103 aa, Vibrio cholerae
  PEPTIDE (1)..(103): >tr|Q9KUT0|Q9KUT0_VIBCH Ribosomal protein L21 - Vibrio cholerae. (103 aa)
MYAVFQSGGKQHRVSEGQTLRLEKLDVETGATVEFDKVLLVANGEDIKVGAPLVEGGKVVAEVVQHGRGDKVKIVKFRRRKHSRKQQGHRQWFTEVKITGINA

SEQ ID NO: 6 - protein, 267 aa, Vibrio cholerae
  PEPTIDE (1)..(267): >sp|Q9KTY5|SUHB_VIBCH Inositol-1- monophosphatase (EC 3.1.3.25) (IMPase) (Inositol-1- phosphatase) (I-1-Pase) - Vibrio cholerae. (267 aa)
MHPMLNIAIRAARKAGNHIAKSLENAEKIQTTQKGSNDFVTNVDKEAEAIIVSTIKSSYPEHCIIAEEGGLIEGKDKEVQWIIDPLDGTTNFVKGFPHFAVSIAVRFRGKTEVACVYDPMTNELFTAQRGAGAQLNNARIRVQPIKDLQGAVLATAFPFKQKQHSESFMKILSAMFVECADFRRTGSAALDLCYLAANRVDGYFELGLKPWDMAAGELIAREAGAIVTDFAGGTDYMQSGNIVASSPRGVKAILQHIRENGNSAILK

SEQ ID NO: 7 - protein, 78 aa, Vibrio cholerae
  PEPTIDE (1)..(76): >sp|Q9KVC8|RL28_VIBCH 50S ribosomal protein L28 -Vibrio cholerae. (76 aa)
MSRVCQVTGKRPAVGNNRSHAKNATKRRFLPNLQTHRFWVESEKRFVKLRLTAKGMRIIDKKGIDAVLVEIRARGENV

SEQ ID NO: 8 - protein, 108 aa, Vibrio cholerae
  PEPTIDE (1)..(108): >sp|P52695|HLYU_VIBCH Transcriptional activator hlyU - Vibrio cholerae. (108 aa)
MPYLKGAPMNLQEMEKNSAKAVVLLKAMANERRLQILCMLLDNELSVGELSSRLELSQSALSQHLAWLRRDGLVNTRKEAQTVFYTLSSTEVKAMIELLHRLYCQANQ

SEQ ID NO: 9 - protein, 394 aa, Vibrio cholerae
  PEPTIDE (1)..(394): >sp|Q9KUZ6|EFTU2_VIBCH Elongation factor Tu-B (EF-Tu-B) - Vibrio cholerae. (394 aa)
MSKEKFERTKPHVNVGTIGHVDHGKTTLTAAICTVLAKVYGGKARDFASIDNAPEERERGITINTSHVEYDTPNRHYAHVDCPGHADYVKNMITGAAQMDGGILVVAATDGPMPQTREHILLGRQVGIPYIIVFMNKCDMVDDEELLELVEMEVRELLSEYDFPGDDLPVIQGSALGALNGEAQWEAKIVELAEALDTYIPEPERAVDMAFLMPIEDVFSIQGRGTVVTGRIERGILKVGDEVAIVGIKETVKTTCTGVEMFRKLLDEGRAGENVGALLRGTKREEVERGQVLAKPGSITPHTKFESEVYVLSKDEGGRHTPFFKGYRPQFYFRTTDVTGSIELPEGVEMVMPGDNVKMVVDLIAPIAMDEGLRFAIREGGRTVGAGVVAKIIA

SEQ ID NO: 10 - protein, 953 aa, Vibrio cholerae
  PEPTIDE (1)..(953): >sp|Q9KP73|SYV_VIBCH Valyl-tRNA synthetase (EC 6.1.1.9) (Valine--tRNA ligase) (ValRS) - Vibrio cholerae. (953 aa)
MEKTYNPTSIEQDLYKTWEEQGYFKPHGDTSKDAYSIMIPPPNVTGSLHMGHAFQDTIMDTLIRCQRMKGKNTLWQVGTDHAGIATQMVVERKIAAEEGKTKHDYGRDAFIDKIWEWKAESGGTITKQLRRLGASVDWDRERFTMDDGFYKAVQEVFVRLYKDDLIYRGKRLVNWDPKLHTAISDLEVENKETKGHMWHFRYPLADGVKTADGKDYIVVATTRPETMLGDTGVAVNPEDPRYKDLIGKEIILPIVGRRIPIVGDEHADMEKGTGCVKITPAHDFNDYEVGKRHQLPMINILTFDANIRDAAEVFNSNGEASNAYGTEIPAKYQGMERFAARKAIVAEFEELGLLQEIKDHDLTVPYGDRGGVVIEPMLTDQWYVRAGILAKPAVEAVENGDIQFVPKQYENMYFSWMRDIQDWCISRQLWWGHRIPAWYDEQGNVFVGRNEEEVRAENNIAADVALRQDDDVLDTWFSSALWTFGTLGWPEKTPELKVFHPTDVLVTGFDIIFFWVARMIMMTMHFCKDEDGKAQVPFKTVYVTGLIRDENGDKMSKSKGNVLDPIDMIDGIDLESLVAKRTGNMMQPQLAAKIEKNTRKTFENGIEAYGTDSLRFTLAAMASTGRDINWDMKRLEGYRNFCNKLWNASRYVLMNTEEQDCGFAAGAELEYSLADKWIESQFELAAKEFNGHIDNFRLDMAANTLYEFIWNQFCDWYLELTKPVLWKGTEAQQRATRRTLITVLEKTLRLAHPVIPYITETIWQSVKPLVDGVEGDTIMLQALPQYDVANFNQEALDDIEWVKAFITSIRNLRAEYDINPGKPLEVMLKAANEQDAARIEANKPVLVSLAKLESIRVLADGEATPACATALVGKSELMIPMAGLIDKDAELDRLAKEIAKTQGEIARIEGKLGNEGFVAKAPEAVITKEREKLAGYQEALVKLEQQKATIAAL

SEQ ID NO: 11 - protein, 886 aa, Vibrio cholerae
  PEPTIDE (1)..(886): >tr|Q9KPF4|Q9KPF4_VIBCH Pyruvate dehydrogenase, E1 component - Vibrio cholerae. (886 aa)
MSDMKHDVDALETQEWLAALESVVREEGVERAQYLLEQVLEKARLDGVDMPTGVTTNYINTIPAAQEPAYPGDTTIERRIRSIIRWNAIMIVLRASKKDLELGGHMASFQSSAAFYETCFNHFFRAPNEKDGGDLVYYQGHISPGIYARAFVEGRLTEEQLDNFRQEVDGKGLPSYPHPKLMPEFWQFPTVSMGLGPISAIYQARFLKYLNGRGLKDTTAQRVYAFLGDGEMDEPESRGAISFAAREKLDNLCFLINCNLQRLDGPVMGNGKIIQELEGLFRGAGWNVVKVIWGNGWDKLLAKDTTGKLLQLMNETIDGDYQTFKAKDGAYVREHFFGKYPETAALVADMTDDEIFALKRGGHESSKLYAAFKNAQDTKGRPTVILAKTVKGYGMGDAAEGKNIAHQVKKMDMTHVLAMRNRLGLQDLISDEEVKNLPYLKLEEGSKEFEYLHARRKALHGYTPQRLPNFTGEFIVPALEDFKPLLEEQSREISSTMAYVRSLNILLKDKNIGQNIVPIIADEARTFGMEGLFRQIGIYNPHGQNYTPQDRDIVSYYKEATSGQVLQEGINELGAMSSWVAAATSYSTNNLPMIPFYIYYSMFGFQRVGDMAWMAGDQQARGFLLGATAGRTTLNGEGLQHEDGHSHILAGTVPNCISYDPTFAYEVAVILQDGIRRMYGEQENVFYYLTLMNESYAHPAMPAGAEEGIRKGIYKLETHAGNKAKVQLMSSGTIMNEVRKAAQILSEEYGVASDVYSVTSFNELARDGQACDRFNMLHPEAEVKVPYIAQVMGTEPAIAATDYMKNYADQVRAFIPAQSYKVLGTDGFGRSDSRENLRRHFEVNAGYVVVAALNELAKRGEVEKSVVAAAIKKFDIDTEKTNPLYA

SEQ ID NO: 12 - protein, 635 aa, Vibrio cholerae
  PEPTIDE (1)..(635): >tr|Q9KPF5|Q9KPF5_VIBCH Pyruvate dehydrogenase, E2 component, dihydrolipoamide acetyltransferase - Vibrio cholerae. (635 aa)
MKVGKVMAIEIYVPDIGADEVEVTEILVKVGDKVAEEQSLITVEGDKASMEVPASQAGIVKEIKVVAGDKVSTGSLIMVFEAEGAAAAAPAPAPQAAAPVAAAPAAAALKEVQVPDIGGDEVEVTEIMVKVGDVVAEEQSLITVEGDKASMEVPAPFAGTVKEIKIAAGDKVSTGSLIMVFEVAGAAPLAAPVQAAAPAAAAAPAVAALKEVQVPDIGGDEVTVTEIMVNVGDSISEEQSLITVEGDKASMEVPAPFAGTLKEIKVATGDKVKTGSLIMVFEVAGAAPVAAPVQAAAAPAAAPAQAATPAAAAPATSGEFQENHEYSHASPVVRRLAREFGVNLAKVKGSGRKNRILKEDVQNYVKEALKRLESGAQAAASGKGDGAALGLLPWPKVDFSKFGDTEVQPLSRIKKISGANLHRNWVMIPHVTQWDNADITELEKFRQEQNAMEAKRDTGMKITPLVFIMKAAAKALEAFPAFNSSLSDDGESLILKKYVNIGIAVDTPNGLVVPVFKDVNKKGIYELSKELAEVSKKARGGKLTAADMQGGCFTISSLGGIGGTAFTPIVNAPEVAILGVSKSEMKPVWNGKEFAPRLQLPLSLSYDHRVIDGAEGARFITYLNECLSDIRRLVL

SEQ ID NO: 13 - protein, 172 aa, Vibrio cholerae
  PEPTIDE (1)..(172): >tr|Q9KR12|Q9KR12_VIBCH Peptidoglycan-associated lipoprotein - Vibrio cholerae. (172 aa)
MQLNKVLKSLLIALPVLAVTACSSSDDAANSGSQTNQSAVSTVDSNGLNAQGQLTEQELKEQALRENQTIYFAFDNATIASDYEAMLAAHAAYLVKNPSLRVTIEGHADERGTPEYNIALGERRAQAVAKYLEALGVQAGQLSIVSYGEEKPLVLGQSEEAYAKNRRAVLVY

SEQ ID NO: 14 - protein, 1297 aa, Vibrio cholerae
  PEPTIDE (1)..(1297): >sp|Q9KTN2|PUR4_VIBCH Phosphoribosylformylglycinamidine synthase (EC 6.3.5.3) (FGAM synthase) (FGAMS) (Formylglycinamide ribotide amidotransferase) (FGARAT) (Formylglycinamide ribotide synthetase)
MRILRGSPALSEFRVNKLLTACREQQLPVTGIYAEFMHFADLKAELNPQELEKLEKLLTYGPTIQEHEPQGLLLLVTPRPGTISPWSSKATDIAHNCGLHGIKRLERGTAYYVEAETALTAAQIATLEALLHDRMMEVVFAELTDAQQLFSVAEPAPMSQVDVLAGGRRALEEANVSLGLALAEDEIDYLVESFTKLGRNPNDIELMMFAQANSEHCRHKIFNADWTIDGVKQDKSLFKMIKNTFEQTPDYVLSAYKDNAAVMTGSTVGRFFPDPESRQYTYHHEDAHILMKVETHNHPTAISPWPGASTGSGGEIRDEGATGIGGKPKAGLVGFTTSNLRIPGFEQPWESDFGKPSRIVNALDIMLEGPLGGAAFNNEFGRPNLLGYFRTYEEKVTSHAGEEVRGYHKPIMIAGGMGNIRAEHIQKKEIPVGAKLIVLGGPAMNIGLGGGAASSMASGQSAEDLDFASVQRENPEMERRCQEVIDRCWQLGDKNPIAFIHDVGAGGISNALPELVNDGDRGGKFQLRNVPNDEPGMSPLEIWCNESQERYVLAVAAEDMPLFDAICQRERAPYAVVGEATEERHLTLEDSHFANTPIDMPMDILLGKPPKMHREASTLKVSSPALERSGIELNEAVDRVLRLPAVAEKTFLITIGDRSVTGLVARDQMVGPWQVPVANCAVTAASFDSYHGEAMSMGERTPVALLDFGASARLAVGEAITNIAATDIGELKRIKLSANWMSPAGHPGEDAGLYEAVKAVGEELCPALGITIPVGKDSMSMKTKWQENGEQKEVTSPLSLIITAFARVEDIRKTVTPQLRTDLGETSLILIDLGNGQNRLGATALAQVYKQLGDKPADVDNAAQLKGFFDAVQTLVRNDKLVAYHDKGDGGLLVTLAEMAFAGHCGIKANIETLGDDALAALFNEELGAVVQVKNDELNAVLATLAAHGLEACAHVIGEVEASDRLLITCGEEVLIERSRTELRTIWAEMTHKMQALRDNSACADQEFAAKQDNRDPGLNAKLTYDVQADVAAPYIAKGVRPKMAILREQGVNSHVEMAAAFDRAGFDAVDVHMSDILTGQTVLDAYQGLVACGGFSYGDVLGAGEGWAKSILFNAQAREQFEQFFQRKDTFSLGVCNGCQMLSNLRDLIPGAELWPRFVRNESDRFEARFSLVEVQKSPSLFFSEMAGSRMPIAVSHGEGRVEVRDAQHLAAIEQSGTVAIRFVDNFGQPTQAYPSNPNGSPNAITGLTTQDGRVTIMMPHPERVFRTVANSWHPDNWGENGAWMRMFQNARKYFG

SEQ ID NO: 15 - protein, 110 aa, Vibrio cholerae
  PEPTIDE (1)..(110): >sp|Q9KNY9|RL22_VIBCH 50S ribosomal protein L22 - Vibrio cholerae. (110 aa)
MEAIAKHNFARISPQKARLVADQIRGKSVDQALELLTFSNKKAAELVKKVLESAIANAEHNEGADIDDLRVAKIFVDEGPVMKRIMPRAKGRADRILKRSSHITVVVADR

SEQ ID NO: 16 - protein, 233 aa, Vibrio cholerae
  PEPTIDE (1)..(233): >sp|Q9KV33|RL1_VIBCH 50S ribosomal protein L1 - Vibrio cholerae. (233 aa)
MAKLTKRMRTIRAKVDVTKEYDINEAVALLKELATAKFVESVDVAVNLGIDARKSDQNVRGATVLPHGTGRDIRVAVFTQGANAEAAKAAGAELVGMEDLADLVKKGEMNFDVVIASPDAMRVVGQLGTILGPRGLMPNPKVGTVTPNVAEAVKNAKAGQVRYRNDKNGIIHTTIGKVTFEADQLKENLEALLVALKKAKPSSAKGVFVKKVSISTTMGAGVAVDQNTLSAQV

SEQ ID NO: 17 - protein, 330 aa, Vibrio cholerae
  PEPTIDE (1)..(330): >sp|Q9KP08|RPOA_VIBCH DNA-directed RNA polymerase alpha chain (EC 2.7.7.6) (RNAP alpha subunit) (Transcriptase alpha chain) (RNA polymerase alpha subunit) - Vibrio cholerae. (330 aa)
MQGSVTEFLKPRLVDIEQISTTHAKVTLEPLERGFGHTLGNALRRILLSSMPGCAVTEVEIEGVLHEYSTKEGVQEDILEILLNLKGLAVRVAEGKDEVFITLNKSGSGPVVAGDITHDGDVEIVNPEHVICHLTSDNAAIAMRIKVERGRGYVPASARIHTEEDERPIGRLLVDATFSPVDKIAYSVEAARVEQRTDLDKLVIDMETNGTLEPEEAIRRAATILAEQLDAFVDLRDVRVPEEKEEKPEFDPILLRPVDDLELTVRSANCLKAEAIHYIGDLVQRTEVELLKTPNLGKKSLTEIKDVLASRGLSLGMRLENWPPASIAED

SEQ ID NO: 18 - protein, 274 aa, Vibrio cholerae
  PEPTIDE (1)..(274): >sp|Q9KNY7|RL2_VIBCH 50S ribosomal protein L2 - Vibrio cholerae.(274 aa)
MAIVKCKPTSAGRRHVVKVVNADLHKGKPYAPLLEKNSKNGGRNNNGRITVRHIGGGHKQHYRLVDFKRTKDGIPAKVERLEYDPNRSANIALVLYADGERRYIIAPKGLQAGDVIQSGPDAPIKAGNAMPMRNIPVGSTIHNVELTPGKGAQLARSAGAYAQLVARDGAYVTLRLRSGEMRKVLSEGRATIGEVGNAEHMLRELGKAGAARWRGVRPTVRGVVMNPVDHPHGGGEGRTSGGRHPVSPWGVPTKGYKTRSNKRTDKYIVRRRNK

SEQ ID NO: 19 - protein, 504 aa, Vibrio cholerae
  PEPTIDE (1)..(504): >tr|Q9KT99|Q9KT99_VIBCH Amidophosphoribosyltransferase - Vibrio cholerae.(504 aa)
MCGIVGIVGTTPVNQSIYDALTVLQHRGQDAAGICTIESNRFRLRKANGLVRDVFEAKHMQRLQGNVGIGHVRYPTAGSSSASEAQPFYVNSPFGITLAHNGNLTNANQVRQKLFEKDRRHVNTTSDSEVLLNVLAHEIDTVKGNVTADDVFRAISNVHRTIRGAYAVAAMIIGHGMIAFRDPHGIRPLCLGKREVNGQLEYMVASESVALDAVGFDFVRDVAPGEAIYATFDGELYTKQCADNPALNPCIFEFVYFARPDSFIDKISVYSARVEMGKRLGERIKNDYSDLDIDVVIPIPETSCDIALQIAQAIDKPYRQGFVKNRYVGRTFIMPGQQQRKKSVRRKLNAIRSEFKDKNVLLVDDSIVRGTTSEQIIEMARDSGAKKVYIVSAAPEIRFPNVYGIDMPSANELIAHGRDNDAICKQIGADALIFQTLEDLVEAVRCGNPDIVKFEASVFNGEYVTGDIDQQYLDFLESMRSDDAKVQREIQQDLANLELHNEGA

SEQ ID NO: 20 - protein, 366 aa, Vibrio cholerae
  PEPTIDE (1)..(366): >tr|Q9KS36|Q9KS36_VIBCH Spermidine/putrescine ABC transporter, periplasmic spermidine/putrescine-binding protein - Vibrio cholerae. (366 aa)
MTEVILFFFATPKRATFSLELTSMKKLATLLAGSACALSLLSGTAAAEDKELVFMNWGPYINSGILEQFTKETGIKVIYSTYESNETLYAKLKTHNQGYDLVVPSTYFVAKMRDEGMLQKIDKSKLSNFKNLDSNYLDKPYDPNNDYSIPHVVAITGLAVNTDMYDPNDFQSWGDLWKPELKGQLMLMDDTREVFHIALRKLGYSGNSTDDKQIDEAYAELQKLMPNVLVFNSDNPAAPYLSGEVGLGMLWNGSAAAAQKEGLPIKLVFPKEGGIGWVDNFAISSGAKNVDAAHKMIDFLLRPEIAEQISRDTGYLTGVKASNDKFQDVAPLFPSQEDLDRVEWQSAVGDKTVKYEEYFMKLKAGQ

SEQ ID NO: 21 - protein, 635 aa, Vibrio cholerae
  PEPTIDE (1)..(635): >sp|P22359|HTPG_VIBCH Chaperone protein htpG (Heat shock protein htpG) (High temperature protein G) - Vibrio cholerae. (635 aa)
MSETATTNKETRGFQSEVKQLLHLMIHSLYSNKEIFLRELISNASDAVDKLRFQALSHPDLYQGDAELGVKLSFDKDKNTLTISDNGIGMTRDEVIENLGTIAKSGTAEFFSKLSQEQSKNSQLIGQFGVGFYSAFIVADAVTVRTRAAGSAPADAVQWYSKGEGEYTVETINKESRGTDIILHLREEGKEFLSEWRLRDVISKYSDHIGIPVYIQTSVMDEEGKATEETKWEQINKAQALWTRAKSEVTDEEYKEFYKHVSHDFADPLVWSHNKVEGKNDYTSLLYIPAKAPWDLFNREHKHGLKLYVQRVFIMDDAAQFMPSYLRFVRGLIDSNDLPLNVSREILQDNKITQSLRQACTKRVLTMLERMASNDADNYQKFWKEFGLVMKEGPAEDFANREKIASLLRFASTHIDSAEQTISLASYVERMKEGQDKIYYLTADSYTAAKNSPHLEQFKSKGIEVILMFDRIDEWLMNYLPEFEGKAFQSITKAGLDLSQFEDEAEKEKHKETEEQFKSVVERLKGYLGSRVKEVRTTFKLANTPAVVVTDDYEMGTQMAKLLAAAGQPVPEVKYILEVNPEHALVKRMADEADEQTFGRWAEVLLGQAMLAERGSMEDPSQFLGAVNQLLAPSH

SEQ ID NO: 22 - protein, 347 aa, Vibrio cholerae
  PEPTIDE (1)..(347): >tr|Q9ZA92|Q9ZA92_VIBCH Rod shape-determining protein MreB - Vibrio cholerae. (347 aa)
MFKKLRGMFSNDLSIDLGTANTLIYVKGQGIVLDEPSVVAIRQDKGRGGKTVAAVGHAAKQMLGRTPGNISAIRPMKDGVIADFYVTEKMLQHFIRQVHDNSVLKPSPRVLVCVPCGSTQVERRAIRESALGAGAREVYLIDEPMAAAIGAGLRVSEPTGSMVIDIGGGTTEVAVISLNGVVYSSSVRIGGDRFDEAIINYVRRNYGSLIGEATAEKIKHEIGSAYPGDDVQEIEVRGRNLAEGVPRSFTLNSNEILEALQEPLTGIVSAVMVALEQCPPELASDISENGMVLTGGGALLKDLDRLLMEETGIPVVIADDPLTCVARGGGKALEMIDMHGGDLFSEE

SEQ ID NO: 23 - protein, 109 aa, Vibrio cholerae
  PEPTIDE (1)..(109): >sp|Q9KT50|Y1055_VIBCH Hypothetical UPF0133 protein VC1055 - Vibrio cholerae.
MFGKGGMGNLMKQAQQMQERMQKLQEEIANMEVTGESGAGLVKVTVTGSHSVRRVNIDESLMEDDKEMLEDLIAAAFNDAARRIEETQKEKMASITGGMQLPPGMKMPF

SEQ ID NO: 24 - protein, 209 aa, Vibrio cholerae
  PEPTIDE (1)..(209): >sp|Q9KNY4|RL3_VIBCH 50S ribosomal protein L3 - Vibrio cholerae. (209 aa)
MIGLIGRKVGMTRVFTEDGVSIPVTVVEVEANRVSQVKTLETDGYAAIQVTTGSKKANRVTKPEAGHFAKAGVEAGRGLWEFRLENGEEFAVGSELTVELFNEVKKVDVTGTSKGKGFQGAVKRWNFRTQDMTHGNSLSHRAPGSIGQCQTPGRVFKGKKMAGHMGAERVTTQNLEIVRVDAERNLLLIKGAVPGATGGNVIVKPAVKA

SEQ ID NO: 25 - protein, 242 aa, Vibrio cholerae
  PEPTIDE (1)..(242): >sp|Q9KPV2|RS2_VIBCH 30S ribosomal protein S2 - Vibrio cholerae. (242 aa)
MASVSMRDMLTAGVHFGHQTRYWNPKMKQFIFGARNRVHIINLEKTVPMFNEALAELAKVGEKKGKVLFVGTKRAASESVKEAALASNQYYVNNRWLGGMLTNWKTVRQSIKRLKELEVQSTDGTFDKLTKKEALMRTREMEKLEKSLGGIKDMGGLPDALFVIDADHEHIAIKEANNLGIPVFAVVDTNSSPDGVDYIIPGNDDAIRAVQLYLNAAAQAINEGRNKDVAAVAEKDGFVEAE

SEQ ID NO: 26 - protein, 556 aa, Vibrio cholerae
  PEPTIDE (1)..(556): >tr|Q9KQT3|Q9KQT3_VIBCH Ribosomal protein S1 - Vibrio cholerae. (556 aa)
MTESFAQLFEEFLNETQFQQGTIVKGTVVAIENGYVLVDAGLKSESAIPAEQFKNAAGELEVQVGSQVDVALDAVEDGFGETQLSREKAKRHEAWIVLEKAYEEAATVIGIINGKVKGGFTVELNGIRAFLPGSLVDVRPIRDTAHLENKELEFKVIKLDQKRNNVVVSRRAVIESESSVERDELLETLQEGTEVKGIVKNLTDYGAFVDLGGVDGLLHITDMAWKRVKHPSEIVNVGDEILVKVLKFDRDRTRVSLGLKQLGEDPWVAIAKRYPEGHKLSGRVTNLTDYGCFVEIEEGVEGLVHVSEMDWTNKNIHPSKVVNVGDEVEVMVLDIDEERRRISLGLKQCKANPWQSFAEAQAKGDKVTGKIKSITDFGIFIGLEGGIDGLVHLSDISWNVPGEEAVREFKKGDEISAVVLAVDAERERISLGIKQMENDPFNAYVSDNKKGALVNGTVTAVDAKGATIELEDGVEGYIRASEVSRDRIEDASLILNVGDKVEAKFTGVDRKNRVINLSIKAKDEAEEQEVMATLNKQDEAAFGNAMADAFKAAKGE

SEQ ID NO: 27 - protein, 1341 aa, Vibrio cholerae
  PEPTIDE (1)..(1341): >sp|Q9KV30|RPOB_VIBCH DNA-directed RNA polymerase beta chain (EC 2.7.7.6) (RNAP beta subunit) (Transcriptase beta chain) (RNA polymerase beta subunit) - Vibrio cholerae. (1341 aa)
MVYSYTEKKRIRKDFGTRPQVLDIPYLLSIQLDSFEKFIEQDPEGQYGLEAAFRSVFPIQSYNGNSELQYVSYRLGEPVFDVKECQIRGVTYSKPLRVKLRLVIFDKDAPAGTVKDIKEQEVYMGEIPLMTENGTFVINGTERVIVSQLHRSPGVFFDSDKGKTHSSGKVLYNARIIPYRGSWLDFEFDPKDNLYVRIDRRRKLPASIILRALGKTSAEILDIFFEKVNFEVKDQTLMMELVPERLRGETATFDIEADGKVYVEKGRRVTARHIRQLEKDGVNFIEVPVEYIVGKVSAKDYVNEATGELIITANQEISLEALANLSQAGYKKLEVLFTNDLDHGPFMSETLRVDSTTDRISALVEIYRMMRPGEPPTKEAAESLFESLFFSAERYDLSTVGRMKFNSSIGREDAEEQGTLDEVDIIEVMKKLISIRNGKGEVDDIDHLGNRRIRSVGEMAENQFRVGLVRVERAVKERLSLGDLDNVMPQDLINAKPISAAVKEFFGSSQLSQFMDQNNPLSEVTHKRRISALGPGGLTRERAGFEVRDVHVTHYGRLCPIETPEGPNIGLINSLSAFARCNEYGFLETPYRRVVNGVVTDEVDYLSAIEEGQFVIAQANAKLTEEGGFADELVTARQKGESGLHPREHVDYMDVATNQVVSIAASLIPFLEHDDANRALMGANMQRQAVPTLRSEKPLVGTGIERNVAVDSGVTAVAKRGGVIQSVDASRIVVKVNEEELIPGEAGIDIYNLTKYTRSNQNTCINQRPCVMPGEPVARGDVLADGPSTDLGELALGQNMRIAFMPWNGYNFEDSILVSERVVQDDRFTTIHIQELSCVARDTKLGAEEITADIPNVGEAALSKLDESGIVYIGAEVKGGDILVGKVTPKGETQLTPEEKLLRAIFGEKASDVKDTSLRVPNSVAGTVIDVQVFTRDGVEKDKRALEIEQMQLKEAKKDLTEEFQILEGGLLARVRSVLLAGGYTEAKLGSIERKKWLEQTLENEELQNQLEQLAEQYDELKADFDKKFEAKRRKITQGDDLAPGVLKIVKVYLAVKRRIQPGDKMAGRHGNKGVISKINPVEDMPYDENGQPVDIVLNPLGVPSRMNIGQILEVHLGLAAKGIGDKINQMIKEQQELAKLREFLQKVYDLGDTRQRVDISELSDEDVRTLAHNLRAGLPVATPVFDGAPESSIKAMLELADLPASGQLTLFDGRTGDAFERPVTVGYMYMLKLNHLVDDKMHARSTGSYSLVTQQPLGGKAQFGGQRFGEMEVWALEAYGAAYTLQEMLTVKSDDVNGRTKMYKNIVDGNHAMEPGMPESFNVLLKEIRSLGINIELEDE

SEQ ID NO: 28 - protein, 358 aa, Vibrio cholerae
  PEPTIDE (1)..(358): >tr|Q9KUN7|Q9KUN7_VIBCH Fructose-bisphosphate aldolase, class II - Vibrio cholerae. (358 aa)
MSKIFDFVKPGVISGDDVQKVFEVAKQNGFALPAVNCVGTDSVNGVLEAAAKVKAPVIVQFSNGGAAFFAGKGLKLEGQQAQILGAIAGAKYVHTVAEAYGVPVILHTDHAAKNLLPWIDGLLDAGEKHFAETGKPLFSSHMIDLSEESLEENIEISAKYLERMAKMNMTLEIELGCTGGEEDGVDNSDMDSSELYTSPEDVAYAYEKLNAISHRFTIAASFGNVHGVYKPGNVVLTPTILRDSQKYVSEKFGLPENSLNFVFHGGSGSSLEEIRESISYGVIKMNIDTDTQWATWDGIRQYEAKNHDYLQGQIGNPTGEDAPNKKYYDPRVWLRAGQAAMVARLEKAFSDLNAIDVL

SEQ ID NO: 29 - protein, 433 aa, Vibrio cholerae
  PEPTIDE (1)..(433): >sp|Q9KPC5|ENO_VIBCH Enolase (EC 4.2.1.11) (2-phosphoglycerate dehydratase) (2-phospho-D- glycerate hydro-lyase) - Vibrio cholerae.
MSKIVKVLGREIIDSRGNPTVEAEVHLEGGFVGMAAAPSGASTGSREALELRDGDKSRFLGKGVLKALAAVNGPIADALVGKDAKDQATIDQIMIDLDGTENKSNFGANAILAVSLANAKAAAAAKGMPLYEHIAELNGTPGVFSMPLPMMNIINGGEHADNNVDIQEFMIQPVGAKTLKEAVRMGAEVFHNLAKVLKSKGYNTAVGDEGGFAPNLKSNAEALEVIAEAVAAAGYKLGTDITLAMDCAASEFYDAEKKEYNLKGEGRIFTSNGFSDFLEELTEKFPIVSIEDGLDESDWEGFAYQTEKLGKKIQIVGDDLFVTNTKILKRGIDNGIANSILIKFNQIGSLTETLAAIKMAKDAGYTAVISHRSGETEDATIADLAVGTAAGQIKTGSMSRSDRVAKYNQLIRIEEALGSRAPFNGLKEVKGQA

SEQ ID NO: 30 - protein, 206 aa, Vibrio cholerae
  PEPTIDE (1)..(206): >sp|Q9KP07|RS4_VIBCH 30S ribosomal protein S4 - Vibrio cholerae. (206 aa)
MARYLGPKLKLSRREGTDLFLKSGVRAIDTKCKIDNAPGVHGARRGRLSEYGVQLREKQKVRRIYGVLEKQFRNYYKEAARLKGNTGENLLQLLEGRLDNVVYRMGFGATRAEARQLVSHKAILVNGKVVNVPSFNVAANDVVAVREKAKKQSRIKAALEVAEQREKPTWIEVDVNTMEGTFKRMPERSDLSADINEQLIVELYSK

SEQ ID NO: 31 - protein, 92 aa, Vibrio cholerae
  PEPTIDE (1)..(92): >sp|Q9KRK0|RL25_VIBCH 50S ribosomal protein L25 - Vibrio cholerae.
MKFEAVLRTDLGKGASRRLRNTGYFPAIVYGGEAAPVSISLNHDDVMNQMDKPEFYEAIVLVIDGQEVKVKPQDVQRHAYKPKVEHMDFIRI

SEQ ID NO: 32 - protein, 55 aa, Vibrio cholerae
  PEPTIDE (1)..(55): >sp|Q9KQH3|RL32_VIBCH 50S ribosomal protein L32 - Vibrio cholerae. (55 aa)
AVQQNRKTRSRRGMRRSHDALTAAALSVDATSGETHLRHNVTAEGYYRGKKVINK

SEQ ID NO: 33 - protein, 71 aa, Vibrio cholerae
  PEPTIDE (1)..(71): >sp|P66532|RS21_VIBCH 30S ribosomal protein S21 - Vibrio cholerae. (71 aa)
MPVVKVRENEPFDVALRRFKRSCEKAGILSEVRRREHYEKPTTVRKRAKAAAQKRHAKKLARENARRVRLY

SEQ ID NO: 34 - protein, 373 aa, Vibrio cholerae
  PEPTIDE (1)..(373): >sp|Q06952|GMD1_VIBCH Probable GDP-mannose 4,6-dehydratase (EC 4.2.1.47) (GDP-D-mannose dehydratase) - Vibrio cholerae. (373 aa)
MNKKVALITGITGQDGSYLAEFLLEKGYEVHGIKRRSSLFNTQRVDHLYKDPHEEDVNFKLHYGDLTDSSNLTRILAEVQPDEVYNLGAQSHVAVSFQSPEYTADVDAIGTLRLLEAIRFLGLTKKTKFYQASTSELYGLVQEIPQKETTPFYPRSPYAVAKMYAYWITINYRESYGIYACNGILFNHESPRRGETFVTRKITRGMANIAQGLEKCLFMGNLDALRDWGHAKDYVKMQWMMLQQDEPRDFVIATGVQYSVREFIDMSARELGIELEFVGKGVDEKAVVKSVIGTKAPAIKVGDIIVAVDPAYFRPAEVETLLGDPSLAKKELGWVPEITLQQMVSEMVASDLEQAQSHALLKKHGYNVNVSVE

SEQ ID NO: 35 - protein, 510 aa, Vibrio cholerae
  PEPTIDE (1)..(510): >sp|Q9KU60|SYK_VIBCH Lysyl-tRNA synthetase (EC 6.1.1.6) (Lysine--tRNA ligase) (LysRS) - Vibrio cholerae. (510 aa)
MTDAVQNEINQEQIAQEENKLIAERRSKLDHIRKNCKANGHPNSFRRDSLAGDLQKKFGEKSKEELEALNHVVSIAGRVMAKRGPFLVIQETSGRIQAYASKEVQQELKDKYQGLDIGDIIGVQGALHKSGKGDLYVNMEQFQLLTKALRPLPEKFHGLTDQEMRYRQRYVDLIVNENSRNAFIVRSKVMSAIRNFMISKQFMEVETPMMHVIPGGASARPFITHHNALDMPMYLRIAPELYLKRLVVGGFDRVFEINRNFRNEGLSPRHNPEFTMMEFYMAYADYKDLMDLTEELLSSVALEVLGSTSMPYGEHTVEFGGKYARMSMFDAIKHYNPNHAQIQALTEEDIQNRDLMVSIAKSVHVDVEPFWTCGQLLEEIFGETAEPKLMQPTFITGYPADISPLARRSDDNPFFTDRFEFFIGGREVANGFSELNDAEDQDARFKAQVEAKESGDDEAMFYDADYITALEHGLPPTAGQGIGIDRLVMLLTNTHTIRDVILFPAMRPQQ

SEQ ID NO: 36 - protein, 207 aa, Vibrio cholerae
  PEPTIDE (1)..(207): >tr|Q9KTZ9|Q9KTZ9_VIBCH Antioxidant, AhpC/Tsa family - Vibrio cholerae (207 aa)
MLRSKKMVLVGRKAPDFTAAAVLGNGEIVDNFNFAEFTKGKKAVVFFYPLDFTFVCPSELIAFDNRYEDFLAKGVEVIGVSIDSQFSHNAWRNTAVENGGIGQVKYPLVADVKHEICKAYDVEHPEAGVAFRGSFLIDEEGMVRHQVVNDLPLGRNIDEMLRMVDALNFHQTHGEVCPAQWEAGKAGMEASPKGVAAFLSQYSADLK

SEQ ID NO: 37 - protein, 129 aa, Vibrio cholerae
  PEPTIDE (1)..(129): >sp|P66367|RS11_VIBCH 30S ribosomal protein S11 - Vibrio cholerae. (129 aa)
MAKQPTRARKRVRKQVADGVAHIHASFNNTIVTITDRQGNALAWATAGGSGFRGSRKSTPFAAQVAAERCAEMAKEYGLKNLEVMVKGPGPGRESTVRALNAAGFRITNIVDATPIPHNGCRPPKKRRV

SEQ ID NO: 38 - protein, 394 aa, Vibrio cholerae
  PEPTIDE (1)..(394): >sp|Q9KV37|EFTU1_VIBCH Elongation factor Tu-A (EF-Tu-A) - Vibrio cholerae. (394 aa)
MSKEKFERTKPHVNVGTIGHVDHGKTTLTAAICTVLAKVYGGKARDFASIDNAPEERERGITINTSHVEYDTPNRHYAHVDCPGHADYVKNMITGAAQMDGGILVVAATDGRMPQTREHILLGRQVGIPYIIVFMNKCDMVDDEELLELVEMEVRELLSEYDFPGDDLPVIQGSALGALNGEAQWEAKIVELAEALDTYIPEPERAVDMAFLMPIEDVFSIQGRGTVVTGRIERGILKVGDEVAIVGIKETVKTTCTGVEMFRKLLDEGRAGENVGALLRGTKREEVERGQVLAKPGSITPHTKFESEVYVLSKDEGGRHTPFFKGYRPQFYFRTTDVTGSIELPEGVEMVMPGDNVKMVVDLIAPIAMDEGLRFAIREGGRTVGAGVVAKIIA

SEQ ID NO: 39 - protein, 319 aa, Vibrio cholerae
  PEPTIDE (1)..(319): >tr|Q9KPW8|Q9KPW8_VIBCH Acetyl-CoA carboxylase, carboxyl transferase alpha subunit - Vibrio cholerae. (319 aa)
MSLNFLDFEKPIVELETKIQALRDVSRHSTSASVDLDKELEQLEKKSLELKKKIFSDLGAWQVAQLARHPQRPYTLDYLKHIFTEFDELAGDRAYADDKAIVGGIARLEGRSVMVIGHQKGRETREKVKRNFGMPKPEGYRKALRLMEMAERFNMPIITFIDTAGAYPGVGAEERGQSEAIAKNLKVMSGLKVPVICNVVGEGGSGGALAIGVGDYVNMLQYSTYSVISPEGCASILWRDSDKAPQAAEAMGLIAPRLKELELIDEIIEEPLGGAHRDHKQTAENVKATLLRQLADLDALDHENLLERRYQRLMNYGYC

SEQ ID NO: 40 - protein, 330 aa, Vibrio cholerae; identical to SEQ ID NO: 17
  PEPTIDE (1)..(330): >sp|Q9KP08|RPOA_VIBCH DNA-directed RNA polymerase alpha chain (EC 2.7.7.6) (RNAP alpha subunit) (Transcriptase alpha chain) (RNA polymerase alpha subunit) - Vibrio cholerae. (330 aa)

SEQ ID NO: 41 - protein, 513 aa, Vibrio cholerae
  PEPTIDE (1)..(513): >sp|Q9KNH3|ATPA_VIBCH ATP synthase alpha chain (EC 3.6.3.14) - Vibrio cholerae. (513 aa)
MQLNSTEISDLIKQRIESFNVVSEARNEGTIVSVSDGIIRIHGLADVMQGEMIELPGNRYALALNLERDSVGAVVMGPYADLREGMKVTGTGRILEVPVGPELLGRVVNTLGEPIDGKGPIGAKQTSPVEVIAPGVIDRKSVDQPVQTGYKSVDSMIPIGRGQRELIIGDRQTGKTALAIDAIINQKNSGIYSIYVAIGQKASTIANVVRKLEEHGALKNTIVVVASASESAALQYLAPYSGCAMGEYFRDRGEDALIVYDDLSKQAVAYRQISLLLRRPPGREAFPGDVFYLHSRLLERAARVNEEYVERFTKGEVKGKTGSLTALPIIETQAGDVSAFVPTNVISITDGQIFLQTELFNAGVRPAVDPGISVSRVGGAAQTKIVKKLSGGIRTALAAYRELAAFAQFSSDLDEATKRQLTHGQKVTELMKQKQYAPMSVFDQALVIFAAERGYLTDVELNKVLDFEAALLSYARAHYAELAAQIDKTGAYNDEIEAQLKKLVDDFKATQTW

SEQ ID NO: 42 - protein, 1401 aa, Vibrio cholerae
  PEPTIDE (1)..(1401): >sp|Q9KV29|RPOC_VIBCH DNA-directed RNA polymerase beta′ chain (EC 2.7.7.6) (RNAP beta′ subunit) (Transcriptase beta′ chain) (RNA polymerase beta′ subunit) - Vibrio cholerae. (1401 aa)
MKDLLNFLKAQHKTEEFDAIKIGLASPDMIRSWSFGEVKKPETINYRTFKPERDGLFCARIFGPVKDYECLCGKYKRLKHRGVICEKCGVEVTQTKVRRDRMGHIELASPVAHIWFLKSLPSRIGLLMDMPLRDIERVLYFEMYVVTEPGMTDLERGQMLTEEEYLDRLEEWGDEFTAKMGAEAIKDLLASMDLPAEAEQMREELDTTNSETKRKKLTKRLKLVEAFVASGNKPEWMILTVLPVLPPDLRPLVPLDGGRFATSDLNDLYRRVINRNNRLKRLLELAAPDIIVRNEKRMLQESVDALLDNGRRGRAITGSNKRPLKSLADMIKGKQGRFRQNLLGKRVDYSGRSVITVGPYLRLHQCGLPKKMALELFKPFIYSKLETRGLATTIKAAKKMVEREEAVVWDILDEVIREHPVLLNRAPTLHRLGIQAFEPVLIEGKAIQLHPLVCAAYNADFDGDQMAVHVPLTLEAQLEARTLMMSTNNILSPASGDPIIVPSQDVVLGLYYMTREKINAKGEGMYLTGPAEAEKAYRTKTAELHARVKVRITETIKHENGKLTTETKMIDTTVGRAMLWQIVPKGLPYSLVNQKLGKKQISNLLNEAYRKLGLKDTVIFADQIMYTGFAYAALSGVSVGIDDMVVPAAKYTEIAEAEEEVREIQEQFQSGLVTAGERYNKVIDIWASTNDRVAKAMMENLSSEQVINRQGEQEKQESFNSIYMMADSGARGSAAQIRQLAGMRGLMARPDGSIIETPITANFKEGLNVLQYFISTHGARKGLADTALKTANSGYLTRRLVDVAQDVVVTEHDCGTLEGVVMTPHIEGGDVKVALTELALGRVVSEDILKPGTDEVLIPRNTLLDEKWCKVINDNSVDQIKVRSVVTCDSDFGCCAQCYGRDLARGHLVNQGEAVGVIAAQSIGEPGTQLTMRTFHIGGAASTAAAENSIQAKNNGSVKLHNAKFVTNKDGKLVITSRASELTIIDEFGRTKEKHKLPYGSMLSKADGDAVAAGETVANWEAHTMPIITEVAGRVQFVDMIDGVTVSRQTDDLTGLSSSEVTEAAARPAAGKDMRPAIKLVDANGKDVLIPGTDMPAQYFLPGKAIVNLDDGAEVNVGDTLARIPQKSGGNKDITGGLPRVADLFEARKPKEPAILAEHSGTVSFGKETKGKRRLIITRDSGDTYEEMIPKHRQLNVFEGERIERGDVIADGPESPHDILRLRGIHAVTTYIANEVQEVYRLQGVKINDKHIETIVRQMLRKCTITFAGDSEFLPGETVEYSQVKIANRKLVEEGKEPARFERELLGITKASLATESFISAASFQETTRVLTEAAVSGKRDDLRGLKENVIVGRLIPAGTGFAYHQDRQAKRAQEQQGPSAEQATDNLAALLNAGFSSDDE

SEQ ID NO: 43 - protein, 82 aa, Vibrio cholerae
  PEPTIDE (1)..(82): >sp|Q9KUG0|RS16_VIBCH 30S ribosomal protein S16 - Vibrio cholerae. (82 aa)
MVTIRLARHGAKKRPFYQIVVADSRNSATGRFIEKVGFFNPTATGQEEGLRLDLDRVNHWVSQGASLSDRVAQLVKTAQKAA

SEQ ID NO: 44 - protein, 122 aa, Vibrio cholerae
  PEPTIDE (1)..(122): >sp|Q9KUZ2|RS6_VIBCH 30S ribosomal protein S6 - Vibrio cholerae. (122 aa)
MRHYEIVFMVHPDQSEQVAGMIERYTGSITEAGGKIHRLEDWGRRQLAYPINKLHKAHYVLMNVEADQAVVDELETAFRFNDAVLRNMIMRTKAAITEPSIMLKAREERVKRDEMKFDADVE

SEQ ID NO: 45 - protein, 85 aa, Vibrio cholerae
  PEPTIDE (1)..(85): >sp|Q9KTD6|PTHP_VIBCH Phosphocarrier protein HPr (Histidine-containing protein) - Vibrio cholerae. (85 aa)
MYEKQVEITAENGLHTRPAAQFVKEAKAFDADITVTSNGKSASAKSLFKLQTLGLVKGTVVTISAEGPQAKEAVEHLVALMDQLH

SEQ ID NO: 46 - protein, 894 aa, Vibrio cholerae
  PEPTIDE (1)..(894): >tr|Q9KQG5|Q9KQG5_VIBCH Alcohol dehydrogenase/acetaldehyde dehydrogenase - Vibrio cholerae.
MPVTNLAELDALVARVKAAQAEFATFSQEQVDKIFRAASLAANQARIPLAQMAVEESGMGIVEDKVIKNHFASEFIYNKYKDEKTCGILEEDDNLGTMTIAEPVGIICGIVPTTNPTSTAIFKSLISLKTRNGIIFSPHPRAKNSTNAAAKLVLDAAIAAGAPKDIIGWIDQPSVELSNALMKHDGIALILATGGPGMVKAAYSSGKPAIGVGAGNVPVVIDETADIKRAVASILMSKTFDNGVVCASEQAAIVVSEVYDEVKERFATHKAHVLSKADADKVRKVLLIDGALNAKIVGQPAAAIAEMAGVKVPADTKVLVGEGLGKVSYDDEFAHEKLSPTLGLFRADNFEDAVAQAVTMVEIGGIGHTSGLYTNQDVNADRIRYFGDKLKTARILVNIPTTHGGIGDLYNFNVAPSLTLGCGSWGGNSISENVGPKHLINKKTVAKRAENMLWHKLPKSIYFRRGSLPIALSDLEGKKRAFLVTDRFLFNNGYADDVVALLKAQGMEVQTFFEVEADPTLSVVEKGAAAMQSFQPDVILALGGGSPMDAAKIMWVMYEHPDTHFEELAMRFMDIRKRIYKFPKMGKKAELVCITTTSGTGSEVTPFAVVTDDKTGAKYPLADYELTPQMAIVDANLVMNMPKSLTAFGGYDAVTHALEAYVSVLANEYSDGQALQALKMLKEYLPSSYANGAKDPIAREKVHNAATIAGIAFANAFLGVCHSMAHKIGAEFHLPHGLANALLIANVVRYNANDNPTKQTAFSQYDRPQARRRYAEVADHLGLSQPGDRTAQKIERLLTWLDELKVNLDIPKSIQAAGVAEADFLAKVDELAVEAFDDQCTGANPRYPLIAELKEVLLASYYGKPFVEGQTFEGTTVIVKKADQEAAKAPKAKK

SEQ ID NO: 47 - protein, 200 aa, Vibrio cholerae
  PEPTIDE (1)..(200): >sp|Q9KNY5|RL4_VIBCH 50S ribosomal protein L4 - Vibrio cholerae. (200 aa)
MELMVKGANALTVSETTFGREFNEALVHQVVVAYAAGARQGTRAQKTRSEVSGGGAKPWRQKGTGRARAGTIRSPIWRTGGVTFAAKPQDHSQKVNKKMYRGAMKSILSELVRQERLIVVENFSVEAPKTKALVAKLKELELNDVLIVTGEVDENLFLAARNLYKVDVRDVTGIDPVSLIAFDKVLMTAAAVKQVEEMLA

SEQ ID NO: 48 - protein, 429 aa, Vibrio cholerae
  PEPTIDE (1)..(429): >sp|Q9KV81|PUR2_VIBCH Phosphoribosylamine- -glycine ligase (EC 6.3.4.13) (GARS) (Glycinamide ribonucleotide synthetase) (Phosphoribosylglycinamide synthetase) - Vibrio cholerae. (429 aa)
MQVLIIGSGGREHALAWKVAQNPQVDTIYVAPGNAGTALEHKVQNVNIGITDIPALVAFAQDKAIELTIVGPEAPLVIGVVDAFRAAGLPIFGPTQGAAQLEGSKAFTKDFLARHNIPTAAYANFTEIEPALAYVREKGAPIVVKADGLAAGKGVIVAMTLQEAEDAIQDMLAGNAFGSAGSRVVVEEFLDGEEASFIVMVDGENVLPMATSQDHKRVGDADTGPNTGGMGAYSPAPVVTQDVHDRVMREVIDPTVRGMAAEGNTYTGFLYAGLMIDSTGAPKVIEYNCRFGDPETQPIMMRLQSDLVELCQAAIAGKLDQVESKWDPRASIGVVLAAGGYPGDYAKGEVISGLPTQESAGQKVFHAGTETQGDQVVTNGGRVLCATALGNTVLEAQQRAYQLADQIHWNGMFCRRDIGYRAIAREQAK

SEQ ID NO: 49 - protein, 645 aa, Vibrio cholerae
  PEPTIDE (1)..(645): >sp|Q9KVS8|THIC_VIBCH Thiamine biosynthesis protein thiC - Vibrio cholerae. (645 aa)
MSNRKQARLEAKRFIDTLSVEPYPNSQKSYLLGSRPDIRVPVREITLSDTLVGGSKDAPIFEPNEPICVYDTSGVYTDPSHDIDLYKGLPKLREEWIEERRDTHILPSMSSHFARERLADETLDELRYGHLPRIRRAMGQHRVTQLHYARQGIITPEMEFVAIRENSRRLAHQDPSLLQQHAGQNFGAHLPDLITPEFVRREIAEGRAIIPCNINHPESEPMIIGRNFLVKVNANIGNSSVSSSIEEEVEKLVWATRWGADTVMDLSTGRNIHETREWILRNSPVPIGTVPMYQALEKVNGVAENLTWEVMRDTLLEQAEQGVDYFTIHAGLLLRYVPMTAKRVTGIVSRGGSIIAKWCLSHHQENFLYTHFREICEICAQYDVALSLGDGLRPGSIADANDEAQFAELRTLGELTQIAWEYDVQVMIEGPGHVPMHLIKANMDEQLKHCHEAPFYTLGPLTTDIAPGYDHITSGIGAAMIGWFGCAMLCYVTPKEHLGLPNKEDVKTGLITYKLAAHAADLAKGHPGAQIRDNALSKARFEFRWEDQFNLALDPVTARAFHDETLPQESGKVAHFCSMCGPKFCSMKISQEVRDYANNQTLDTTVIDLVMPAESIQLAMQDKSREFLASGAELYHPLVKEPIEE

SEQ ID NO: 50 - protein, 156 aa, Vibrio cholerae
  PEPTIDE (1)..(156): >sp|Q9KUZ8|RS7_VIBCH 30S ribosomal protein S7 - Vibrio cholerae. (156 aa)
MPRRRVIGQRKILPDPKFKSELLAKFVNILMVDGKKSTAEKIVYTALDTMAEKSGKDHLAVFEEALENVRPAVEVKSRRVGGSTYQVPVEVRPVRRNALAMRWLVEAARKRGEKSMAARLAAEMLDAAENKGSAVKKREDVHRMAEANKAFAHYRW

SEQ ID NO: 51 - protein, 103 aa, Vibrio cholerae
  PEPTIDE (1)..(103): >sp|Q9KNY3|RS10_VIBCH 30S ribosomal protein S10 - Vibrio cholerae. (103 aa)
MQNQRIRIRLKAFDYKLIDASTAEIVETAKRTGAQVRGPIPLPTRKERFTVLISPHVNKDARDQYEIRTHKRLIDIVEPTDKTVDALMRLDLAAGVDVQISLG

SEQ ID NO: 52 - protein, 288 aa, Vibrio cholerae
  PEPTIDE (1)..(288): >tr|Q9KNH4|Q9KNH4_VIBCH ATP synthase F1, gamma subunit - Vibrio cholerae. (288 aa)
MAGAKEIRTKIGSVKSTQKITKAMEMVAASKMRRSQDAMESSRPYAQTIRKVIGHVANASLEYRHPYLEEREAKRVGYIIISTDRGLCGGLNINLFKKAITDMQTWKEKGAQIELAIIGSKATAFFNNSGAKVAAQVSGLGDSPSLEDLIGSVGVMLKKYDKGELDRLYLVFNQFVNTMVQKPKIDQLLPLPKSDSEDMQRDHMWDYIYEPEPKPLLDALLLRFIESQVYQGVVENLACEQAARMVAMKAATDNASNLIDDLQLVYNKARQAAITQELSEIVGGAAAV

SEQ ID NO: 53 - protein, 343 aa, Vibrio cholerae
  PEPTIDE (1)..(343): >tr|Q9KPN7|Q9KPN7_VIBCH 2,3,4,5-tetrahydropyridine-2-carboxylate N-succinyltransferase - Vibrio cholerae. (343 aa)
MAYFALGLATATKNRDGKIIEAFFPTPILAPSDALVAALAPIAGYQEGNQALDITAAQSAQLAAVFAAHQQAASAAFADKAANAKQPLVLVILASDDKPQSVAEGYLKLQLISHRLVKPHGTVLDGIFGLLHNIAWTNEGPIDLPELAERQIEARLAGRVLTVDCVDKFPKMVDYVVPAGIRIADTSRVRLGAHVGEGTTVMHEGFINFNAGTTGVSMVEGRISAGVVVGNGSDIGGGASIMGTLSGGGKVVVSIGENSLLGANAGLGFPLGDRCTVESGLYVTAGTKVRTLDKDGNQVDIVKARDLAGVSDLLFRRNSLTGQIECLANKSAVELNSELHKNN

SEQ ID NO: 54 - protein, 466 aa, Vibrio cholerae
  PEPTIDE (1)..(466): >sp|Q9KSF9|SYN_VIBCH Asparaginyl-tRNA synthetase (EC 6.1.1.22) (Asparagine--tRNA ligase) (AsnRS) - Vibrio cholerae. (466 aa)
MTYAPVNDVLSGKLAVDSEVTVRGWIRTRRDSKAGISFLAIYDGSCFNPIQAVVPNNLNNYDNEVLKLTTGCSVEVTGKIVESPASGQAFELAASDVKVVGWVEDADTYPMAKTRHSIEYLREVAHLRPRTNVIGAVARVRNCLAQAIHRFYHEQGYFWVSAPLITASDAEGAGEMFRVSTLDMENLPRTDAGKVDYNQDFFGKETFLTVSGQLNAEAYACAISKVYTFGPTFRAENSNTSRHLAEFWMVEPEVAFADLNTVAKLAEDMLKYVFKAVLAERRDDLEFFNDRINNEVIARLEQFVESDFAQVDYTDAIEILKNCGKTFEFPVEWGIDLASEHERFLAEEHFKAPVIVKNYPKDIKAFYMRMNEDGKTVAAMDVLAPGIGEIIGGSQREERLDILDARMREFGIDPEHMDWYRDLRRYGTVPHAGFGLGFERLVSYVTGMGNVRDVIPFPRTPRSASF

SEQ ID NO: 55 - protein, 90 aa, Vibrio cholerae
  PEPTIDE (1)..(90): >sp|Q9KNM3|RPOZ_VIBCH DNA-directed RNA polymerase omega chain (EC 2.7.7.6) (RNAP omega subunit) (Transcriptase omega chain) (RNA polymerase omega subunit) - Vibrio cholerae. (90 aa)
MARVTVQDAVEKIGNRFDLVLVAARRARQMQSGGKDALVPEENDKPTVIALREIEEGLITKDVLDARERQEQQEQEAAELAAVSSIMHNR

SEQ ID NO: 56 - protein, 544 aa, Vibrio cholerae
  PEPTIDE (1)..(544): >sp|Q9KNR7|CH601_VIBCH 60 kDa chaperonin 1 (Protein Cpn60 1) (groEL protein 1) - Vibrio cholerae. (544 aa)
MAAKDVRFGNDARVKMLEGVNILADAVKVTLGPKGRNVVLDKSFGAPTITKDGVSVAREIELEDKFQNMGAQMVKEVASQANDAAGDGTTTATVLAQAIVNEGLKAVAAGMNPMDLKRGIDKAVIAAVEELKALSVPCADTKAIAQVGTISANSDSSVGNIIAEAMEKVGRDGVITVEEGQALQDELDVVEGMQFDRGYLSPYFINNQESGSVELDNPFILLVDKKISNIRELLPVLEGVAKASRPLLIVAEDVEGEALATLVVNNMRGIVKVAAVKAPGFGDRRKAMLQDIAILTGGVVISEEIGLELEKATLEDLGQAKRVSITKENSTIIDGAGDQAAIQGRVAQIRQQIEEATSDYDKEKLQERVAKLAGGVAVIKVGAATEVEMKEKKDRVEDALHATRAAVEEGVVAGGGVALIRAASKLSSLVGDNEEQNVGIRVALRAMEAPLRQIVKNAGDEESVVANNVRAGEGNYGYNAATGVYGDMIEMGILDPTKVTRSALQFAASVAGLMITTEAMITELPKKDAPAMPDMGMGGMGGMM

SEQ ID NO: 57 - protein, 130 aa, Vibrio cholerae
  PEPTIDE (1)..(130): >sp|Q9KUF0|RS9_VIBCH 30S ribosomal protein S9 - Vibrio cholerae. (130 aa)
MAENQYYGTGRRKSSAARVFIKPGSGNIVINKRSLEEYFGRPTSCMVVKQPLELVDMVEKLDLYITVKGGGISGQAGAIRHGITRALMEYDESLRPVLRAAGYVTRDARRVERKKVGLRKARRRPQFSKR

SEQ ID NO: 58 - protein, 787 aa, Vibrio cholerae
  PEPTIDE (1)..(787): >tr|Q9KQY1|Q9KQY1_VIBCH Formate acetyltransferase - Vibrio cholerae. (787 aa)
MKSVLLPSGIAGSTAKTVRVFSTHKGRYVMAEQFAKAWEGFAAGDWQNEVNVRDFIQKNYTPYEGDESFLVSEGTEATNKLWAKVMEGIRQENSTHAPVDFDTSLISTITAHDAGYINKDLEKIVGLQTDAPLKRAIIPNGGIRMVEGSCKAYGRELDPQVSKIYSEYRKTHNAGVFDIYTPEILACRKSGVLTGLPDAYGRGRIIGDYRRVALYGIDFLMKDKLAQFKSLQEKFENGEDLQMTMQLREEIAEQHRALGQMKQMAAKYGYDISRPAETAQEAIQWTYFGYLAAVKSQNGAAMSLGRTSTFLDVYIERDMKAGKITEVEAQEMIDHFVMKLRMVRFLRTPEYDELFSGDPIWATESMGGMGLDGRTLVTRTNFRFLNSLYTMGPSPEPNITVLWSEQLPEGFKKFCAKVSIDTSSIQYENDDLMRPDFNNDDYAIACCVSPMVIGKHMQFFGARANLAKTLLYVINGGVDEKLKIQVGPKMPKITDEVLDFDDVWGKLDHFMGWLATQYVTALNAIHYMHDKYSYEAALMALHDRDVRRTMACGIAGLSVAADSLSAIKYAKVKPIRDEDGVAIDFEIEGDYPKFGNNDSRVDDIACELVERFMNKIRSLKTYRNAVPTQSILTITSNVVYGKKTGNTPDGRRAGAPFAPGANPMHGRDEKGAVASLTSVGKLPFAHAKDGISYTFSIVPNALGKDENSQRANLAGLMDGYFHHEAGIEGGQHLNVNVLNRDTLLDAVKHPEKYPQLTIRVSGYAVRFNSLTAEQQQDVIARTFTESL

SEQ ID NO: 59 - protein, 320 aa, Vibrio cholerae
  PEPTIDE (1)..(320): >sp|Q9KNP2|K6PF_VIBCH 6-phosphofructokinase (EC 2.7.1.11) (Phosphofructokinase) (Phosphohexokinase) - Vibrio cholerae. (320 aa)
MIKKIGVLTSGGDAPGMNAAIRGVVRTALGAGLEVFGIYDGYQGLYEDRIKQLDRSSVSDVINRGGTFLGSARFPQFREVEVREKAIENLKKHGIEALVVIGGDGSYMGAKKLTEMGFPCIGLPGTIDNDIAGTDYTIGYLTALNTVIEAIDRLRDTSSSHQRISIVEIMGRHCGDLTLTSAIAGGCEYIITPETGLNMEQLISNIKDGIAKGKKHAIIALTELMMDANKLAKEIESATGRETRATVLGHIQRGGKPTAFDRVLASRMGNYAVHLLMEGHGGRCVGIQKEQLVHHDIIDAIENMKRPVRTDLYKVAEELF

SEQ ID NO: 60 - protein, 130 aa, Vibrio cholerae
  PEPTIDE (1)..(130): >sp|Q9KNZ8|RS8_VIBCH 30S ribosomal protein S8 - Vibrio cholerae. (130 aa)
MSMQDPISDMLTRIRNGQAANKVAVKMPSSKLKVAIAALLKAEGYIADFAVEGEVKAELEITLKYFQAKPVIEQIKRVSRPGLRVYKKKDELPSVMGGLGVAVVSTSKGLMSDRAARKAGLGGEIICYVA

SEQ ID NO: 61 - protein, 257 aa, Vibrio cholerae
  PEPTIDE (1)..(257): >sp|Q9KNR1|TPIS_VIBCH Triosephosphate isomerase (EC 5.3.1.1) (TIM) (Triose-phosphate isomerase) - Vibrio cholerae. (257 aa)
MRRPVVMGNWKLNGSKAMVTDLLNGLNAELEGVEGVDVVVAPPAMYLDLAERLIKEGGNKLILGAQNTDTHNSGAYTGDMSPAMLKDFGASHIIIGHSERRDYHKESDEFVAKKFAFLKENGLTPVFCIGETEAQNEAGETEAVCARQINAVIDAYGVEALNGAIIAYEPIWAIGTGKAATADDAQRIHASIRALIAAKDAAVAEQVIIQYGGSVKPENAASYFAQPDIDGALVGGASLDAKGFAAIAKAAAEAKKA

SEQ ID NO: 62 - protein, 176 aa, Vibrio cholerae
  PEPTIDE (1)..(176): >sp|Q9KP34|IPYR_VIBCH Inorganic pyrophosphatase (EC 3.6.1.1) (Pyrophosphate phospho- hydrolase) (PPase) - Vibrio cholerae. (176 aa)
MSLNNVPAGKSLPDDIYVVIEIPANADPIKYEVDKESGAVFVDRFMSAPMFYPCNYGYVNHTLSLDGDPVDVLVPTPYPLIPGSVIRCRPVGVLKMTDESGEDAKVVAVPHTKISKEYDHIQDVNDLPALLKAQITHFFERYKELESGKWVKVDGWEDAASARAEILSSYERAQNK

SEQ ID NO: 63 - protein, 116 aa, Vibrio cholerae
  PEPTIDE (1)..(116): >sp|Q9KUF7|RL19_VIBCH 50S ribosomal protein L19 - Vibrio cholerae. (116 aa)
SNIIKALEQEQMKQDLPQFAPGDTVVVQVKVKEGDRERLQAFEGIVIAIRNRGLHSAFTVRKISNGEGVERTFQTHSPVVDSIEVKRRGAVRRAKLYYLRDLSGKAARIKEKLAKK

SEQ ID NO: 64 - protein, 394 aa, Vibrio cholerae; identical to SEQ ID NO: 38
  PEPTIDE (1)..(394): >sp|Q9KV37|EFTU1_VIBCH Elongation factor Tu-A (EF-Tu-A) - Vibrio cholerae. (394 aa)

SEQ ID NO: 65 - protein, 389 aa, Vibrio cholerae
  PEPTIDE (1)..(389): >tr|Q9KUV8|Q9KUV8_VIBCH MSHA biogenesis protein MshN - Vibrio cholerae. (389 aa)
MGMEQTMSVVNEALAKSAQRSHSRLSNIERIDVAKPKARPAWLWVMLGFGVSLAVGGWSISLQSVDTIPSSAEVVRPEVPSPTQKTTQQSIALYQAPVNSEALVKNETLVQKETLPKNETSVKNAVLAKVEGSPTSLKQTANRQREQTALGSELNAEPVLNLVDNSEPSFFEEEVSSLPSSSPVMIVEQVSLTPEQLAQKALQRAQKAMESNELQTAVSAYTEALRYTPHDEMARQKLAALYYGKGDGRKAFDLMQAGIERNPDGEVLRLALAKLLVKEKQEASALVPLAYLPSQPSIEYLSLRAALAQKTKQDEIARESYQQLTEKDPNNGRWWLGLAIQQERALQWPAAQHAYQQALNKVGLSSQSQAFIHQRLQLLASLEETQSAN

SEQ ID NO: 66 - protein, 642 aa, Vibrio cholerae
  PEPTIDE (1)..(642): >sp|Q9KMN7|SYT_VIBCH Threonyl-tRNA synthetase (EC 6.1.1.3) (Threonine--tRNA ligase) (ThrRS) - Vibrio cholerae. (642 aa)
MPIITLPDGSQRHFDNPVSTLEVAQSIGPGLAKATIAGRVNGARVDACDLIEHDASLEIITTKDEVDGLEIVRHSCAHLLGHALKQLYPNAKMAIGPTIDSGFYYDIDLEQSLSQEDLEKIEARMVELAKTKYAVVKKKVSWQEARDTFESRGESYKMEILDENVARDDRPGLYHHEEYIDMCRGPHVPHMGFCQNFKLLNIAGAYWRGNSDNKMLQRIYGTAFHDKKALQAHLTRLEEAAKRDHRKIGKQLDLFHMQQEAPGMVFWHHNGWSIFRDLEIFIRQKLNEYGYQEVKGPLMMDRVLWERSGHWDKYADAMFTTSSENREYAIKPMNCPGHIQIFNQGLKSYRDLPLRMAEFGSCHRNEPSGSLHGIMRVRGFTQDDAHIFCTEDQIQQEVTSCIKMVYDTYTTFGFQNIVVKLSTRPEKRVGSDEIWDKSEQALIDSLKAMDIPFEIQEGEGAFYGPKIEFTLYDCLDRAWQCGTVQLDFNLPTRLGATYVGESNERLIPVMIHRAILGSLERFIGILIEEYAGFFPTWLAPEQAVVVNITDKQADYAHEVAQKLQKCGIRAKADLRNEKIGFKIREHTLKRVPYMLVCGDQEMEAGEIAVRTRKGKDLGKFKLDDFIAHIQAEIASRKLNLEE

SEQ ID NO: 67 - protein, 645 aa, Vibrio cholerae; identical to SEQ ID NO: 49
  PEPTIDE (1)..(645): >sp|Q9KVS8|THIC_VIBCH Thiamine biosynthesis protein thiC - Vibrio cholerae. (645 aa)

SEQ ID NO: 68 - protein, 311 aa, Vibrio cholerae
  PEPTIDE (1)..(311): >tr|Q9KQQ6|Q9KQQ6_VIBCH Methylenetetrahydrofolate dehydrogenase/methenyltetrahydrofolate cyclohydrolase - Vibrio cholerae. (311 aa)
MPVDVIKNICDNVRKNFRSKPVRKVMTAQNIDGTLISQTVRSEVAARVKARVQAGLRAPGLAVVLVGEDPASQVYVGSKRRACEEVGFVSKSFDLPATASEEALLSLVEELNNDPQIDGILVQLPLPAGMDTTKVLESIHPEKDVDGFHPYNVGRLAQRIPKLRSCTPKGIITLLERYNIPLRGKHAVIVGASNIVGRPMTLELLLAGCTTTTCHRFTQDLEGHIRQADILVVAVGKPNFIPGAWIKEGAVVVDVGINRLDTGKLVGDVEYDVARTRASFITPVPGGVGPMTVASLIENTMMACEQFHTQG

SEQ ID NO: 69 - protein, 446 aa, Vibrio cholerae [sequence omitted]

SEQ ID NO: 70 - protein, 635 aa, Vibrio cholerae
  PEPTIDE (1)..(635): >sp|O34241|DNAK_VIBCH Chaperone protein dnaK (Heat shock protein 70) (Heat shock 70 kDa protein) (HSP70) - Vibrio cholerae. (635 aa)
MGKIIGIDLGTTNSCVAVLDGDKPRVIENAEGERTTPSVIAYTDGETLVGQPAKRQAVTNPQNTLFAIKRLIGRRFEDEEVQRDIKIMPYKIVKADNGDAWVEAKGQKMAAPQVSAEVLKKMKKTAEDFLGEPVTAAVITVPAYFNDAQRQATKDAGRIAGLEVKRIINEPTAAALAYGLDKQGGDRTIAVYDLGGGTFDISIIEIDEVEGEKTFEVLSTNGDTHLGGEDFDNRMINYLVDEFKKDQGIDLRNDPLAMQRLKEAAEKAKIELSSAQQTDVNLPYITADATGPKHMNIKVTRAKLEALVEDLVQRSLEPLKVALADADLSVNDITDVILVGGQTRMPMVQKKVAEFFGKEPRKDVNPDEAVAVGAAVQGGVLAGEVKDVLLLDVTPLSLGIETMGGVMTKLIEKNTTIPTKANQVFSTAEDNQSAVTIHVLQGERKQAMYNKSLGQFNLEGINPAPRGMPQIEVIFDLDADGILHVSAKDKQTGKEQKITIQASGGLSDAEIEKMVQEAEANKEADKKFEELATARNQADQMIHATRKQITEAGEALPADEKAKIETAINELETAKKGEDKAEIDAKVQALMAAAQKLMEIAQQQAQAQGANAGQSSAKEDDVVDAEFEEVNDDKK

SEQ ID NO: 71 - protein, 117 aa, Vibrio cholerae
  PEPTIDE (1)..(117): >sp|Q9KP00|RL18_VIBCH 50S ribosomal protein L18 - Vibrio cholerae. (117 aa)
MDKKASRIRRATRARRKIAELGATRLVVHRTPRHVYAQVIAANGSEVIAAASTVEKAIREQVKYTGNIDAAKAVGKAVAERALEKGVTVVAFDRSGFQYHGRVAALADSAREAGLKF

SEQ ID NO: 72 - protein, 345 aa, Vibrio cholerae
  PEPTIDE (1)..(345): >tr|Q9KMH9|Q9KMH9_VIBCH Hypothetical protein VCA0374 - Vibrio cholerae. (345 aa)
MILRQLNRIDMSSFDFPKYQANQEVGNTGEAFFEHFVTKELGCIYRKVHREHDFGIDGYIDIVNNGVVSGRSVAVQIKCGDSYISKVTSGGIKYLGDNKHLNFLINHNVPVILVVLNSDCTKGYWVEFSVNATNPSSKSWWIEIPFENQLGTLDMTSLCDIAGIAEDYSEDIKVSWAINSAMDEQSDISFVLSKEEVEELDLSGINQMMERYFRTKDLLISNRNKTVFWIFGYDDDSRELYEIPEVRRWFKFTLEQGVPWFYLLDVGADHMSLPINMYSCCNISVNKLSCGSKSVVISSVSDINNWVEVNFENLNRFADEKKIPDNILKEVSEKVITSVHRLVAG

SEQ ID NO: 73 - protein, 331 aa, Vibrio cholerae
  PEPTIDE (1)..(331): >tr|Q9KQJ8|Q9KQJ8_VIBCH Glyceraldehyde 3-phosphate dehydrogenase - Vibrio cholerae. (331 aa)
MTIKVGINGFGRIGRFVFRAAQERNDIEIVGINDLIDVDYMAYMLKYDSTHGRFNGTVEVKDGNLIVNGKTVRVTAERNPADLKWGEIGVDVVAEATGLFLTDETARKHIEAGAKKVVLTGPSKDKTPMFVMGVNHKTYAGQDIVSNASCTTNCLAPIAKVLNDNFGIESGLMTTVHATTATQKTVDGPSAKDWRGGRGASQNIIPSSTGAAKAVGVVLPELNGKLTGMAFRVPTANVSVVDLTVNLQKAASYDDIKAAMKAASEGELAGILGYTEDAVVSTDFNGDTRTSIFDADAGIALTDKFVKVVSWYDNEIGYSNKVLDLIAHISK

SEQ ID NO: 74 - protein, 513 aa, Vibrio cholerae; identical to SEQ ID NO: 41
  PEPTIDE (1)..(513): >sp|Q9KNH3|ATPA_VIBCH ATP synthase alpha chain (EC 3.6.3.14) - Vibrio cholerae. (513 aa)

SEQ ID NO: 75 - protein, 128 aa, Vibrio cholerae
  PEPTIDE (1)..(128): >tr|Q9KP09|Q9KP09_VIBCH Ribosomal protein L17 - Vibrio cholerae. (128 aa)
MRHRKSGRQLNRNSSHRKAMFSNMASSLVRHEVIKTTLPKAKELRRVVEPLITLAKTDSVANRRLAFARTRDNEVVAKLFNELGPRFAARQGGYTRILKCGFRAGDKAPMAYIELVDRPEAAVEAAAE

SEQ ID NO: 76 - protein, 233 aa, Vibrio cholerae
  PEPTIDE (1)..(233): >sp|Q9KNZ0|RS3_VIBCH 30S ribosomal protein S3 - Vibrio cholerae. (233 aa)
MGQKVHPNGIRLGIVKPWNATWFANTKDFADNLDGDFKVRQYLSKELANASLSRIVIERPAKSIRVTIHTARPGVVIGKKGEDVEKLRAAVAKIAGVPAQINIAEVRKPELDGQLVADSIASQLERRVMFRRAMKRAVQNAMRLGAKGIKVEVSGRLGGAEIARSEWYREGRVPLHTLRADIDYATSSAHTTYGVIGVKVWIFKGEILGGMPAATEAAEPKADKPKKQRKGRK

SEQ ID NO: 77 - protein, 194 aa, Vibrio cholerae
  PEPTIDE (1)..(94)
MAFELPALPYAKDALEPHISAETLDFHHGKHHNTYVVKLNGLIPGTEFENKSLEEIIKTSTGGIFNNAAQVWNHTFYWHCLSPNGGGEPTGAVAEAINAAFGSFADFKAKFTDSAINNFGSSWTWLVKKADGTLAITNTSNAATPLTEEGVTPLLTVDLWEHAYYIDYRNVRPDYMNGFWALVNWDFVAQNLAK

SEQ ID NO: 78 - protein, 1052 aa, Vibrio cholerae
  PEPTIDE (1)..(1052): >tr|Q9KQG8|Q9KQG8_VIBCH Ribonuclease E - Vibrio cholerae. (1052 aa)
MKRMLINATQKEELRVALVDGQRLFDLDIESPGHESKKANIYKGRITRIEPSLEAAFVDYGAERHGFLPLKEIAREYFPDGYSYQGRPNIKDVLNEGQEVIVQIEKEERGSKGAALTTFISLAGSYLVLMPNNPRAGGISRRIEGDERTELKSALSSLELPQGMGLIVRTAGVGKSAEELEWDLNVLLKHWSAIKQASDSNPAPFLIHQESNVIVRAIRDYLRRDIGEILIDSNSIYERALEHIRLVRPDFVNRVKKYEGEVPLFSHFQIESQIESAFQREVRLPSGGSIVIDPTEALTSIDINSARATKGGDIEETALNTNLEAADEIARQLRLRDLGGLVVIDFIDMTPVRHQREVENRLREAVRVDRARVQIGRISRFGLLEMSRQRLSPSLAEASHHICPRCKGTGVIRDNESLALSVLRLIEEEALKDNTSQVLAVVPVSIASYLLNEKRRSINHIEKAQQVRITIVPNSDMETPHFEVIRVREGEEQDVLSYLIPKKLEAMKEAEGKEVVDVELKPKRIEEPVLKGFAAPAEAVPAPTPKPKAEPQPVAEVQQPAQPGFFSRVFKAIASLFSATPEAAKVEAPVTQNSEQDKPRRERNDRNNDRRRNPRDKNRRRGNEENNERNDKETSNSNRKPQERKPKQERGERNERNDRNERPETERPERQDRRNKRDESKTAKLLEQGRQLAAEAQQEVKAVEPKEEKAAVVKERRQRRKLSKQIRVKDQLAAEELDNLSTAPVDNAFDAPAPVNLPVTDDFADSAQEDNEQDDQKQRRNRRSPRHLRASGQRRRRGRDRRPNPFRLRKGGVASPEMAMGKVMPRYDLAPRPQSRHETAEGAQHVTHVTPTAATAVVESEKVAAPRVLGGVAFPEMAMGKVIVRRETAAVQPEPVVQEPVITDVIAETLIAPVEPVVVEATVIEAPVIEESATETAALETVVAKVAEVEEPATISDEVVAQTTVEVIADKMAEPIKVVKPVTVSATSLTAKAVIQQPYASSPMTKAPGSDDIREVQVNAAPLRAERYQARGAGSQVARNQASAGMSKPQSF

SEQ ID NO: 79 - protein, 466 aa, Vibrio cholerae
  PEPTIDE (1)..(466): >sp|Q9KNH5|ATPB_VIBCH ATP synthase beta chain (EC 3.6.3.14) - Vibrio cholerae. (466 aa)
ATGKIVQIIGAVVDVEFPQSEVPSVYDALNVVDSKERLVLEVQQQLGGGVIRAIVMGSSDGLRRGMTVQNTGAPISVPVGTKTLGRIMNVLGDAIDERGDIGAEEVYSIHRPAPSYEEQSSATELLETGVKVIDLICPFAKGGKIGLFGGAGVGKTVNMMELINNIALQHSGLSVFAGVGERTREGNDFYHEMQEAGVVNVEQPELSKVAMVYGQMNEPPGNRLRVALTGLTMAEKFRDEGRDVLLFIDNIYRYTLAGTEVSALLGRMPSAVGYQPTLAEEMGVLQERITSTKKGSITSVQAVYVPADDLTDPSPATTFAHLDATVVLNRNIAAMGLYPAIDPLDSTSRQLDPLVVGQEHYDVARGVQATLQRYKELKDIIAILGMDELSEADKQVVARARKIERFLTQPYHVAEVFTGDPGVYVPLKETLRGFKGLLAGDYDDIPEQAFMYCGTIDDAIENAKKL

SEQ ID NO: 80 - protein, 517 aa, Vibrio cholerae
  PEPTIDE (1)..(517): >sp|Q9KTW2|GUAA_VIBCH GMP synthase [glutamine-hydrolyzing] (EC 6.3.5.2) (Glutamine amidotransferase) (GMP synthetase) - Vibrio cholerae. (517 aa)
MTKNIHDQRILILDFGSQYTQLVARRVREIGVYCELWSWDVEEADIREFNPDGIILSGGPESVTEANSPRAPQYVFDSGVPVFGVCYGMQTMAEQLGGRVATSDEREFGYAQVKISGESALFKDLDLTQDVWMSHGDKVVEIPADFVKIGETDTCPYAAMANEEKKYYGVQFHPEVTHTKNGLQMLENFVLGVCGCERLWTSESIIEDAVARIKEQVGNDEVILGLSGGVDSSVVAMLVHRAIGSKLTCVFVDNGLLRLNEGEQVMEMFGDKFGLNIIKVDAEERFLKALEGIDEPEAKRKTIGRVFVEVFDEESKKLSNAKWLAQGTIYPDVIESAASKTGKAHVIKSHHNVGGLPDDMKMGLVEPLRELFKDEVRKIGLELGLPYNMLYRHPFPGPGLGVRVLGEVKKEYCDLLRRADAIFIEELHAADLYNKVSQAFTVFLPVRSVGVMGDGRKYDWVVSLRAVETIDFMTAHWAHLPYEFLGKVSNRIINEVNGISRVVYDISGKPPATIEWE

SEQ ID NO: 81 - protein, 512 aa, Vibrio cholerae
  PEPTIDE (1)..(512): >tr|Q9KM26|Q9KM26_VIBCH NAD(P) transhydrogenase, alpha subunit - Vibrio cholerae. (512 aa)
MQIGVPREQLAGETRVAASPSSVEQLIKLGFEVCIESKAGALASFDDAAYTAAGATIGSREEIWACPLILKVNAPSDDEIALLKEGATLVSFIWPAQNPALMEKLSSKNINVLAMDAVPRISRAQALDALSSMANIAGYRAVVEAAHEFGRFFTGQITAAGKVSPAKVFVAGAGVAGLAAIGAAGSLGAIVRAFDVRPEVKEQVQSMGAEFLEVNFQETAGSGDGYAKEMSDEFNRKAAELYAAQAKDVDIIITTALIPGKPAPKLITKEMVDSMKAGSVIVDLAAANGGNCEYTVKDQVITTDNGVKVIGYTDMVGRLPTQSSQLYATNLVNLLKLLCKEKDGNIDINFEDVVLRGVTVVKAGEITWPAPPIQVSAQPQQKAKVQPTKAQKKEPEPTSPVKKLVGLAVGVGLFAWVASVAPAAFLGHFTVFVLACVVGYYVVWNVTHALHTPLMSVTNAISGIIVVGALLQIGQGSGIVTFLAFIAVLIASINIFGGFTVTKRMLEMFRKN

SEQ ID NO: 82 - protein, 280 aa, Vibrio cholerae
  PEPTIDE (1)..(280): >sp|Q9KPV3|EFTS_VIBCH Elongation factor Ts (EF-Ts) - Vibrio cholerae. (280 aa)
MAVTAALVKELRERTGAGMMECKKALVETNGDIELAIENMRKSGAAKAAKKAGNIAAEGTIMIKEGEGIAALVEVNCQTDFVAKDSNFVAFANQVTDAALASKASVEELQAQFEEARVALVAKIGENINIRRVQYVEGEALATYRHGDRIGVVVAGSADVETLKHVAMHVAASRPEFLTPDDVPAEVVAKEREVQVGIAMNEGKSKEIAEKMVEGRMKKFTGEVSLTGQPFVMEPKKTVGEILAEKGATVSAFIRLEVGEGIEKQEGLSFAEEVALAQKG

SEQ ID NO: 83 - protein, 554 aa, Vibrio cholerae
  PEPTIDE (1)..(554): >tr|Q9KTB2|Q9KTB2_VIBCH Asparagine synthetase B, glutamine-hydrolyzing - Vibrio cholerae. (554 aa)
MCSIFGILDIKTDAATLRPIALEMSKKLRHRGPDWSGIYASDKAILAHERLAIVGLNSGAQPLYSPDRKLILAVNGEIYNHKEIRERYKGQYEFQTDSDCEVILALYRDKGADLLEELNGIFAFVLYDEEKDEYLIGRDHIGIIPLYQGHDEHGNFYVASEMKALVPVCKTLSEFPPGSYLSSKDQAVTRYYVRDWNSFDEVKDNVSSKEELTQALEAAVKRQLMTDVPYGVLLSGGLDSSITSAIAKRFAAMRIEDDEKSAAWWPQLHSFAIGLEGAPDLKAAREVAEKIGTVHHEMTYTIQEGLDAIRDVIYHIETYDVTTIRASTPMFLMGRKIKAMGIKMVLSGEGADEIFGGYLYFHKAPNAKEFHEETVRKLLALNLFDCARANKSLAAWGVEGRVPFLDKEFIDVAMRLNPADKMCGNGKMEKHILRECFEHYLPESIAWRQKEQFSDGVGYGWIDTLKATAEAKISDQQMETAKFRFPYNTPTTKEGYVYREIFEELFPLESAARCVPGGPSVACSSAKAIEWDESFKNCIDPSGRAVKAVHKQAY

SEQ ID NO: 84 - protein, 64 aa, Vibrio cholerae
  PEPTIDE (1)..(64): >sp|O68845|RL35_VIBCH 50S ribosomal protein L35 - Vibrio cholerae. (64 aa)
MPKMKNNKGAAKRFKKTAGGIKYKHATKRHILTKRTTKNKRQLRPNAILPKCELAAVARMLPYA

SEQ ID NO: 85 - protein, 681 aa, Vibrio cholerae
  PEPTIDE (1)..(681): >tr|Q9KP17|Q9KP17_VIBCH 2`,3`-cyclic-nucleotide 2`-phosphodiesterase - Vibrio cholerae. (681 aa)
MKPLFHRLLLHLSSCFYFLLLSLILESVMTMMIKPVSLAVLGGLLAMAGPVMADTIKLRIVETTDIHTNLMDYDYYKDKASDQIGLTRAATLVKQARAEVENSVLVDNGDLIQGSPMGDYMAAKGIKAGEVHPVYKAMNPLGYDVGNIGNHEFNYGLEFLKETINDAAFPYINANVFDAKTGQHLFTPYVIKEHSFKDTDGEAHTIKVGYIGFVPPQIMVWDKKNLEGKVTAADIKQTAEKLIPEMKAKGADVIVAIPHSGISTDEYQAGAENSVYYLTQVKGIDAIAFGHSHAVFPGKDFANVPGADIDKGTINGVTAVMPGRWGSHVGVMDLTLEKQQGRWAVTSGQSEARPIFDKANKKALVEADATMVQALQHDHQGTRDFVNQPIGKANDVMYSFLSLVQDDPTVQIVNLAQKDYVERFIQGDPNLDGLPVLSAAAPFKAGGRKDDPNGFTEVEAGELTFRNAADLYLYPNTLVTMKVSGQQLKEWLECSAGQFNQIDVTSSAPQSLINWDGFRTYNFDVIDGVNYQIDVTQPARYDGDCKLINPNSQRIVQLTYQGKPIDTQQTFLIATNNYRAYSNKFPGTGAEFVAFDSPDENRTVVADYIARVSKDKGEVTPSADNNWSFAPISSKTKLDIRFETSPSDKATQFIKQKGQYPMTRVASDDVGFAVYRIDLQK

SEQ ID NO: 86 - protein, 469 aa, Vibrio cholerae
  PEPTIDE (1)..(469): >sp|Q9KNJ2|GLNA_VIBCH Glutamine synthetase (EC 6.3.1.2) (Glutamate--ammonia ligase) - Vibrio cholerae. (469 aa)
MSVENVLSLIQENEVKFVDLRFTDTKGKEQHISIPAHQIDADFFEDGKMFDGSSVAGWKGINESDMVMMPDPSSAVLDPFTEDATLNIRCDILEPATMQGYDRDPRSIAKRAEEYMRSTGIADTVLVGPEPEFFLFDDVKFATNMSGSFFKIDDVEAAWNTGTEYEDGNKGHRPGVKGGYFPVAPVDSSQDIRSAMCLIMEEMGLVVEAHHHEVATAGQNEIATRFNTLTTKADEIQIYKYVVHNVAHAFGKTATFMPKPLVGDNGSGMHVHQSLAKDGVNLFAGDKYGGLSETALYYIGGIIKHARALNAITNPSTNSYKRLVPHYEAPVMLAYSARNRSASIRIPVVPSPKARRIEVRFPDPAANPYLAFAAMLMAGLDGIKNKIHPGEAMDKDLYDLPAEEAAEIPKVAESLQQALQYLDADREFLTAGGVFSDDFIDSYIALKTKDVERVNVAVHPLEFELYYSV

SEQ ID NO: 87 - protein, 123 aa, Vibrio cholerae
  PEPTIDE (1)..(123): >tr|Q9KNZ4|Q9KNZ4_VIBCH Ribosomal protein L14 - Vibrio cholerae. (123 aa)
MIQMQTMLDAADNSGARSVMCIKVLGGSHRRYAHVGDIIKVTVKEAIPRGKVKKGDVLKAVVVRTRKGVRRPDGSVIRFDRNACVLLNNNSEQPIGTRIFGPVTRELRNAKFMKIVSLAPEVL

SEQ ID NO: 88 - protein, 1076 aa, Vibrio cholerae
  PEPTIDE (1)..(1076): >sp|Q9KPH9|CARB_VIBCH Carbamoyl-phosphate synthase large chain (EC 6.3.5.5) (Carbamoyl- phosphate synthetase ammonia chain) - Vibrio cholerae. (1076 aa)
MPKRTDIQSILILGAGPIVIGQACEFDYSGAQACKALREEGYRVILVNSNPATIMTDPEMADATYIEPIHWEVVRKIIEKERPDAILPTMGGQTALNCALALEKHGVLAEFGVEMIGATADAIDKAEDRSRFDKAMKSIGLECPRADTAKSMEEAYKVLDMVGFPCIIRPSFTMGGSGGGIAYNREEFEEICTRGLDLSPTNELLIDESLIGWKEYEMEVVRDKNDNCIIVCAIENFDPMGIHTGDSITVAPAQTLTDKEYQIMRNASLAVLREIGVETGGSNVQFGINPKDGRMVIIEMNPRVSRSSALASKATGFPIAKVAAKLAVGFTLDELMNDITGGATPASFEPTIDYVVTKIPRFNFEKFAGANDRLTTQMKSVGEVMAIGRNQQESLQKALRGLEVGAAGLDEKVDLDAPDALTKIRYELKEAGAERIWYIADAFRAGMSVDGVFNLTNIDRWFLVQIEELVKLEAEVKAGGFAGLNQDVLRKMKRKGFSDARLSKLLGVSENEIRRLRDQYNIHPVYKRVDTCAAEFKSDTAYMYSTYDEECEANPTDKDKIMVLGGGPNRIGQGIEFDYCCVHAALALREDGYETIMVNCNPETVSTDYDTSDRLYFEPVTLEDVLAIVRVEKPKGVIVQYGGQTPLKLARALEAAGVPVIGTSPDAIDRAEDRERFQQAVQRLGLKQPDNATVTAIEQAIEKSREIGFPLVVRPSYVLGGRAMEIVYDEQDLRRYFNEAVSVSNESPVLLDRFLDDATEVDVDAICDGERVVIGGIMEHIEQAGVHSGDSACSLPAYTLSQEIQDKMREQVEKLAFELGVRGLMNIQFAVKDNEVYLIEVNPRAARTVPFVSKATGAPLAKIAARVMVGQTLEQQGFTKEIIPPYYSVKEVVLPFNKFPGVDPLLGPEMRSTGEVMGVGATFAEAYAKAELGCGSVYPEGGRALLSVREGDKQRVVDLASKLVKLGYQLDATHGTAVILGEAGINPRLVNKVHEGRPHILDRIKNHEYTYIVNTASGRQAIEDSKVLRRGALAHKVNYTTTLNAAFATCMSHTADAKASVTSVQELHARVKANQA

SEQ ID NO: 89 - protein, 695 aa, Vibrio cholerae
  PEPTIDE (1)..(695): >sp|Q9KPM5|EFG2_VIBCH Elongation factor G 2 (EF-G 2) - Vibrio cholerae. (695 aa)
MADLSLYRNIGIFAHVDAGKTTTTERILKLTGKIHRLGEVHDGASTMDFMEQEAERGITIQSAATTCFWKGHRFNVIDTPGHVDFTVEVYRSLKVLDGGIGVFCGSGGVEPQSETNWRYANESEVSRLIFVNKLDRMGADFFRVVEQVKKVLGANPLVMTLPIGREDEFVGVVDVLTRQAYVWDDSGLPENFEVKEVPADMVDQVEEYREMMIETAVEQDDELMMAYMEGEEPTVEQIKACIRKGTRDLAFFPTFCGSAFKNKGMQLVLDAVVDYLPSPTEVEPQPLTDPATGEPTGEVATVSVDAPLKALAFKIMDDRFGALTFVRIYSGKIKKGDTILNSATGKTERIGRMVEMHANDRNEVESAQASDIIAIVGMKNVQTGHTLCDPKHECTLEPMIFPTPVISIAVKPKDKNGSEKMGIAIGKMVAEDPSFQVETDEDSGETILKGMGELHLDIKVDILKRTYGVELEVGAPQVAYRETITKAVEDSYTHKKQSGGSGQFGKIDYRIRPGEQNSGFTFKSTVVGGNVPKEFWPAVEKGFKSMMDTGTLAGFPVLDVEVELFDGGFHAVDSSAIAFEIAAKGAFRQSIPKAAPQLLEPIMKVDVFTPEDHVGDVIGDLNRRRGMIKDQEMGLTGVRVKADVPLSEMFGYIGSLRTMTSGRGQFSMEFSHYAPCPNNVAEQVIAEVKERNAKK

SEQ ID NO: 90 - protein, 898 aa, Vibrio cholerae
  PEPTIDE (1)..(898): >sp|Q9KU80|IF2_VIBCH Translation initiation factor IF-2 - Vibrio cholerae. (898 aa)
MTQITVKALSEEIGTPVDRLLEQLADAGMNKAVADHVSEDEKQKLLAHLRKEHGDATGSEPTRLTLQRKTRSTLSVNAGGGKSKNVQVEVRKKRTYVKRSSVEDEATREAEEAAMRAAEEQAKREAEEAAQRAAEEKAKREAEEAAKREAEAKRMAEEKAKRETQAATQPRSDEEKLKQEAARKEAEALKRRQEEEARRKAEEESRRQLEKVRELAEKNGERWSADKETVGDMQENTDYHVTTSRYAREAEDEADLHEEGARRRSTKANKRKMSSRDDNQERDSRPRGGKAGRKGRINKPMSMQHGFDKTAVVAKADVVVGETIVVSELAQKMSVKATEVIKVMMKMGAMATINQVIDQETAQLVAEEMGHKVVLRKENELEEAILSDRDDKFEEVSRAPVVTIMGHVDHGKTSTLDYIRRTHVASGEAGGITQHIGAYHVETPNGMITFLDTPGHAAFTAMRARGAQATDIVVLVVAADDGVMPQTVEAIQHAKAAGVPLIVAVNKIDKDTANPDNVKTELSQYNVMPEEWGGDNMFVHISAKQGTNIDGLLEAILLQAEVLELKAVKQGMASGVVIESRLDKGRGPVATVLVQSGTLRKGDIVLCGQEYGRVRAMRDEVGNEVEEAGPSIPVEILGLSGVPAAGDEATVVRDERKAREVANYRAGKFREVKLARQQKSKLENMFSNMTAGDVAELNIVLKADVQGSVEAIADSLTKLSTDEVKVNIVGSGVGGITETDAVLAAASNAIVVGFNVRADASARRMIEAENIDLRYYSIIYQLIDEVKQAMSGMLSPEFKQEIIGLAEVRDVFKSPKLGAIAGCMVTEGVIKRNAPIRVLRDNVVIYEGELESLRRFKDDVAEVKNGYECGIGVKNYNDVRVGDQIEVFETIEIQRTID

SEQ ID NO: 91 - protein, 256 aa, Vibrio cholerae
  PEPTIDE (1)..(256): >tr|Q9KTQ6|Q9KTQ6_VIBCH Accessory colonization factor AcfC - Vibrio cholerae. (256 aa)
MYFMKSKNRFLLISLLSFSTSVFADVNLYGPGGPHVPLIKVAESFEKSQSKRVNITFGPQATWNDKAKKNADILFGASEHSALAIAEGHSERFSKFNIHPVFMREAIILVKKGNPKNIKGMADLLKPGIGIVVNDGAGVSNTSGTAVWEDSVGRMKNVEKLQAFRSNIHVFAPNSGSARKAFVDGEDIDAWITWVDWAIANPTIGDMVRMEDEYRIYRDFNVVLAKNPSSEAIDFFDYLTKSKDAEAIFQHYGWFK

SEQ ID NO: 92 - protein, 588 aa, Vibrio cholerae
  PEPTIDE (1)..(588): >tr|Q9KQB1|Q9KQB1_VIBCH Succinate dehydrogenase, flavoprotein subunit - Vibrio cholerae. (588 aa)
MTIPVRSFDAVVIGAGGAGMRAALQISEQGLTCALLSKVFPTRSHTVSAQGGITVALGNSHKDDWQWHMYDTVKGSDYIGDQNAIEYMCKNGPESVIELERMGLPFSRFENGTIYQRPFGGQSKEFGGEQAARTAAAADRTGHALLHTLYQQNVKHKTTIFSEWYALDLVKNQDGAVLGCTAICMETGEICYFKAKATVLATGGAGRIYASTTNAHINTGDGVGMALRAGVPMQDMEMWQFHPTGIAGAGVLVTEGCRGEGGYLLNKDGERFMERYAPNAKDLAGRDVVARSMMIEIREGRGCDGPWGPHIKLKLDHLGRDVLESRLPGICELSRTFAHVDPVKEPIPVIPTCHYMMGGVPTQVSGQAIKQDSHGKDVEVQGLFACGEIASVSVHGANRLGGNSLLDLVVFGRATGLHLGETLRAQAEARPATASDIEASLARTMRWENSKGGEDPVVIRKDLQRCMQNNFSVFREGKAMAEGLEELKVIRERLKNAHLADKSTEFNTQRIECLELDNLMETAFSTAVAANYRTESRGAHARFDYPERDDENWLCHSIYNPETEQMSKREVNMTPVYRDPFPPKVRTY

SEQ ID NO: 93 - protein, 336 aa, Vibrio cholerae
  PEPTIDE (1)..(336): >tr|Q9KP35|Q9KP35_VIBCH Fructose-1,6-bisphosphatase - Vibrio cholerae. (336 aa)
MQKMRTLGEFIVEKQHDFPHASGELSSLLASIRLAAKIVNREINKAGLADIIGASGNDNIQGEEQQKLDLYANEKFKAALEARDQVCGVASEEEDEAVAFSKELNKNAKYVVLMDPLDGSSNIDVNVSVGTIFSIYRRVSPVGTPPTQEDFLQPGNKQVAAGYVIYGSSTMLVYTTGNGVNGFTYDPSLGTFYLSHENMRIPENGKIYSINEGNYIRFPTGVKKYIKFCQENVPEEGRPYTSRYIGSLVADFHRNLLKGGIYLYPSTQSHPNGKLRLLYECNPMAFLIEQAGGLASDGARRIMDIKPTELHQRVPFFVGSKNMVHKVETFLETYPD

SEQ ID NO: 94 - protein, 795 aa, Vibrio cholerae
  PEPTIDE (1)..(795): >sp|Q9KSN6|SYFB_VIBCH Phenylalanyl-tRNA synthetase beta chain (EC 6.1.1.20) (Phenylalanine-- tRNA ligase beta chain) (PheRS) - Vibrio cholerae. (795 aa)
MKFSESWLREWVNPAITTDELTHQITMAGLEVDDVLAVAGVFDGVKVGHVVECAQHPDADKLRVTKVDVGEEELLDIVCGAANCRQGLKVAVATVGATLPGDFKIKKAKLRGQPSHGMLCSFSELGIDVESNGIMELAENAPIGMDFRDFLSLNDVTIDVDLTSNRADCFSIRGLAREVGVLNRADVTAPAVNAIVATINDTISIDVKAPAACPRYLGRIVKNVNVQAQTPLWMQEKLRRCGIRSIDPVVDITNFVMLEQGQPMHAFDLAKIEGGIVVRLAEQDEKLTLLDGSEAKLNADTLVIADQQKALAIAGVFGGEHSGVSTDTKDVLLECAFFAPDHIRGRARSYGLHTDSSMRFERGVDYALQHAAMERATQLLVEICGGDVAPVVAAESAADLPKPNQVALRRSKLDKLLGHAIPDADVVEILERLGMQVETTAEGWQATAPTWRFDIAIEQDLVEEVGRIYGYNNIPNQAPVAALNMNLHNEAKLPLKRVRDLLVDRGYQEAITYSFVEPEQQKLVVPGVDALILPNPISAEMSAMRLSLIQGLLNTVVHNQKRQQPRVRLFEYGLRFIPDAAAENGMRQEPMLAGVISGARGEEHWNMETATVDFFDMKGDLEAVLELTAKGKAYSFAATKHPALHPGQAAAIMVDGKAIGVIGTVHPELERKFGLNGRTIVFEIEWNAINTRVIPEAAAISKFPANRRDIALVVDGNIASGDIVEACRVAGGELLKDAKLFDVYVGKGVEEGKKSLAIALTLQSVERTLEEADIAAAVEAIVQAVSAQFGAALRD